(19) United States
(12) Patent Application Publication
(10) Pub. No.: US 2025/0271448 A1
(43) Pub. Date: Aug. 28, 2025

(54) METHODS FOR IDENTIFYING OR QUANTITATING PEPTIDES

(71) Applicant: PrognomiQ, Inc., San Mateo, CA (US)

(72) Inventors: Chi-Hung LIN, Redwood City, CA (US); Bruce WILCOX, Keezletown, VA (US)

(21) Appl. No.: 19/201,215

(22) Filed: May 7, 2025

Related U.S. Application Data
(63) Continuation of application No. PCT/US2023/079433, filed on Nov. 10, 2023.
(60) Provisional application No. 63/383,164, filed on Nov. 10, 2022.
(60) Provisional application No. 63/384,757, filed on Nov. 22, 2022.
(60) Provisional application No. 63/506,804, filed on Jun. 7, 2023.
(60) Provisional application No. 63/506,805, filed on Jun. 7, 2023.

(51) Int. Cl. G01N 33/68; G01N 33/92

(52) U.S. Cl. CPC G01N 33/6848; G01N 33/92; G01N 2440/38; G01N 2458/15; G01N 2560/00

(57) ABSTRACT

Provided are methods comprising combining a biological sample with a double-labeled peptide standard comprising a peptide comprising a first label and comprising a post-translational modification (PTM) comprising a second label; and identifying or measuring, based on the double-labeled peptide standard, an endogenous protein of the biological sample, wherein the endogenous protein comprises the PTM. Further, provided are methods and devices for enriching and/or purifying glycan-modified macromolecules with higher specificity and sensitivity.

### CROSS-REFERENCE

This application is a continuation of International Application No. PCT/US2023/079433, filed Nov. 10, 2023, which claims the benefit of U.S. Provisional Application No. 63/383,164, filed on Nov. 10, 2022; U.S. Provisional Application No. 63/384,757, filed on Nov. 22, 2022; U.S. Provisional Application No. 63/506,804, filed on Jun. 7, 2023; and U.S. Provisional Application No. 63/506,805, filed on Jun. 7, 2023, each of which is incorporated by reference herein in its entirety.

### BACKGROUND

Post-translational modifications (PTMs), such as phosphorylation and glycation, represent an important level of post-translational peptide activity control. As PTMs are associated with changes in mass of the parent peptide, mass spectrometry techniques represent one of the most versatile routes of PTM detection and characterization.

### SUMMARY

In an aspect, the present disclosure describes a method for generating mass/charge data of mass-spectrometry standards of a plurality of glycan-modified macromolecules. The method comprises obtaining a biological sample and subjecting said biological sample to enriching said plurality of glycan-modified macromolecules to generate a plurality of enriched glycan-modified macromolecules, wherein a glycan-modified macromolecule of said plurality comprises a macromolecular backbone and at least one glycan modified macromolecular residue; attaching a macromolecular backbone-linked mass tag or isotopic tag to a glycan-modified macromolecule of the plurality of enriched glycan-modified macromolecules; attaching a glycan-linked isotopic tag to the glycan-modified macromolecule of the plurality of enriched glycan-modified macromolecules to generate the glycan-modified macromolecular standards; and performing a mass spectrometry assay on the glycan-modified macromolecule standards to obtain a plurality of mass/charge ratios or abundances of an ionized species associated with the glycan-modified macromolecular standards.

In some embodiments, the present disclosure provides that the plurality of glycan-modified macromolecules comprises glycolipids. In some embodiments, the present disclosure provides that the plurality of glycan-modified macromolecules comprises glycopeptides. In some embodiments, the present disclosure provides for digesting the glycopeptides with an endoprotease having a defined residue specificity. In some embodiments, the present disclosure provides the endoprotease comprises Trypsin, rLys-C, Lys-C, rAsp-N, or any combination thereof.

In some embodiments, the present disclosure provides subjecting the biological sample to hydrophobic interaction liquid chromatography (HILIC) or lectin affinity chromatography.

In some embodiments, the present disclosure provides that macromolecular backbone-linked isotopic tag comprises an isobaric labeling reagent. In some embodiments, the present disclosure provides that the isobaric labeling reagent comprises an amine-, thiol-, or carbonyl-reactive moiety and an ionizable moiety comprising a stable heavy isotope. In some embodiments, the present disclosure provides that the isobaric labeling reagent comprises a tandem mass tag (TMT) reagent or an isobaric tag for relative and absolute quantitation (iTRAQ) reagent.

In some embodiments, the present disclosure provides the glycan-linked isotopic tag is specific for: a disaccharide linkage type of the enriched glycan-modified macromolecules, or a monosaccharide of the glycan of the enriched glycan-modified macromolecules. In some embodiments, the present disclosure provides treating the plurality of enriched glycan-modified macromolecules with a monosaccharide-specific or linkage-specific glycosidase to remove a predetermined monosaccharide from a glycan of the plurality of enriched glycan-modified macromolecules. In some embodiments, the present disclosure provides comprising treating the plurality of enriched glycan-modified macromolecules with a linkage-specific glycosidase, wherein the linkage-specific glycosidase comprises a glycosidase specific for an α2-3 sialic acid linkage, a β1-4 galactose linkage, a β1-3 galactose linkage, an α1-2 fucose linkage, or an α1-3/4 fucose linkage. In some embodiments, the present disclosure provides the linkage-specific glycosidase comprises α2-3 Neuraminidase S, β1-4 Galactosidase S, β1-3 Galactosidase S, α1-2 Fucosidase, or α1-3,4 Fucosidase, or any combination thereof. In some embodiments, the present disclosure provides the comprising treating the plurality of enriched glycan-modified macromolecules with a monosaccharide-specific glycosidase, wherein the monosaccharide comprises sialic acid, mannose, N-Acetylglucosamine, or N-Acetylgalactosamine. In some embodiments, the present disclosure provides the glucosidase comprises α2-3,6,8 Neuraminidase, α1-2,3,6 Mannosidase, β-N-Acetylglucosaminidase S, or α-N-Acetylgalactosaminidase, or any combination thereof. In some embodiments, the present disclosure provides treating the plurality of enriched glycan-modified macromolecules with a glycosyltransferase to attach the glycan-linked isotopic tag to a glycan of the enriched glycan-modified macromolecules. In some embodiments, the present disclosure provides the glycosyltransferase is configured to perform an α2-6 sialylation, an α2-3 sialylation, a β1-4 galactosylation, a β1-3 galactosylation, an α1-2 fucosylation, an α1-3 fucosylation, a α1-4 fucosylation, a β1-3 N-acetylglucosaminylation, or a β1-6 N-acetylglucosaminylation, or any combination thereof. In some embodiments, the present disclosure provides wherein the glycosyltransferase comprises α2-6 sialyltransferase 1 (ST6Gal1), α2-3 sialyltransferase 3/4 (ST3Gal3, ST3Gal4), β1-4 Galactosyltransferase 1 (β4GalT1), β1-3 Galactosyltransferase 5 (β3GalT5), Fucosyltransferase 1 or 2 (FucT1 or FucT2), Fucosyltransferase 4 or 7 (FucT4 or FucT7), Fucosyltransferase 3 (FucT3), β1-3N-acetylglucosaminyltransferase 2 (B3GNT2), or β1-6 N-acetylglucosaminylation (GCNT2), or any combination thereof. In some embodiments, the present disclosure provides the glycan-linked isotopic tag corresponds to a heavy isotope labeled derivative of the predetermined monosaccharide. In some embodiments, the present disclosure provides the glycan-linked isotopic tag comprises sialic acid or a derivative thereof, galactose or a derivative thereof, fucose or a derivative thereof, N-acetylglucosamine or a derivative thereof, N-acetylgalactosamine or a derivative thereof, mannose or a derivative thereof, glucose or a derivative thereof, or any combination thereof. In some embodiments, the present disclosure provides the isotopic tag comprises sialic acid or a derivative thereof, wherein the sialic acid or a derivative thereof comprises N-Acetylneuraminic acid (Neu5Ac), N-Glycolylneuraminic acid (Neu5Gc), or 2-keto-3-deoxy-D-glycero-D-galacto-nononic acid (KDN).

In some embodiments, the present disclosure provides the glycan-linked isotopic tag comprises a naturally occurring monosaccharide comprising one, two, three, four, five or six 13C or 16O atoms. In some embodiments, the present disclosure provides wherein the plurality of mass/charge ratios or abundances of an ionized species associated with the glycan-modified macromolecular standards comprise mass/charge ratios of precursor ions of the fragments of the glycan-modified macromolecular standards. In some embodiments, the present disclosure provides the plurality of mass/charge ratios or abundances of an ionized species associated with the glycan-modified macromolecular standards comprise mass/charge ratios of product ions of the fragments of the glycan-modified macromolecular standards. In some embodiments, the present disclosure attaching a second macromolecular backbone-linked isotopic tag distinguishable in mass from the macromolecular backbone-linked isotopic tag in conjunction with a second glycan-linked isotopic tag to a second glycan-modified macromolecule of the plurality of enriched glycan-modified macromolecules. In some embodiments, the present disclosure provides the second glycan-linked isotopic tag via treatment with a second linkage-specific glycosidase or second glycosyltransferase different to the linkage-specific glycosidase or the glycosyltransferase. In some embodiments, the present disclosure provides the second glycan-linked isotopic tag is different to the glycan-linked isotopic tag. In some embodiments, the present disclosure provides that the second glycan-linked isotopic tag is not an isobar of the glycan-linked isotopic tag.

In some embodiments, the present disclosure provides for pooling the glycan-modified macromolecule standards with a glycan-modified macromolecule of a second plurality of enriched glycan-modified macromolecules that has been labeled with a second macromolecular backbone-linked isotopic tag distinguishable in mass from the macromolecular backbone-linked isotopic tag, and obtaining a plurality of mass/charge ratios or abundances of a an ionized species associated with fragments of the glycan-modified macromolecule of the second plurality of enriched glycan-modified macromolecules. In some embodiments, the present disclosure provides for pooling the glycan-modified macromolecule standards with a glycan-modified macromolecule of a third plurality of enriched glycan-modified macromolecules that has been labeled with a third macromolecular backbone-linked isotopic tag distinguishable in mass from the macromolecular backbone-linked isotopic tag and the second macromolecular backbone-linked isotopic tag, and obtaining a plurality of mass/charge ratios or abundances of an ionized species associated with of the glycan-modified macromolecule of the third plurality of enriched glycan-modified macromolecules. In some embodiments, the present disclosure provides for the second plurality of enriched glycan-modified macromolecules and the third plurality of enriched glycan-modified macromolecules are derived from distinct second and third biological samples, respectively.

In some embodiments, the present disclosure provides for comparing an intensity of a mass/charge peak corresponding to a glycan-modified macromolecule of the second- and third-pluralities of enriched glycan-modified macromolecules, thereby identifying a difference in glycan abundance in the second and third biological samples. In some embodiments, the present disclosure provides for using a mass offset of a glycan-modified macromolecule standard of the glycan-modified macromolecule standards to identify a site-specific glycan modification of a macromolecule corresponding to the difference in glycan abundance in the second and third biological samples.

In a different aspect, the disclosure provides a set of mass spectrometry standards of a plurality of glycan-modified macromolecules, wherein a glycan-modified macromolecule of the plurality comprises a macromolecular backbone and at least one glycan modified macromolecular residue, comprising: a plurality of glycan-modified fragments of the glycan-modified macromolecules, wherein the plurality of glycan-modified fragments comprise: a macromolecular backbone-linked mass tag, isotopic tag, or isobaric tag; and a glycan-linked isotopic tag.

In some embodiments, the present disclosure provides that the glycan-modified macromolecules comprise glycolipids. In some embodiments, the present disclosure provides that the glycan-modified macromolecules comprise glycoproteins. In some embodiments, the present disclosure provides that the plurality of glycan-modified fragments comprise fragments generated by treatment with an endoproteinase. In some embodiments, the present disclosure provides that the endoproteinase comprises Trypsin, rLys-C, Lys-C, rAsp-N, chymotrypsin, Glu-C, or any combination thereof.

In some embodiments, the present disclosure provides that the plurality of glycan-modified fragments comprises: (i) peptides with C-terminal arginine or lysine; (ii) peptides with C-terminal lysine; (iii) peptides with C-terminal arginine; (iv) peptides with C-terminal tyrosine, phenylalanine, or tryptophan; or (v) peptides with C-terminal glutamate.

In some embodiments, the present disclosure provides that the macromolecular backbone-linked isotopic tag comprises an isobaric labeling reagent. In some embodiments, the present disclosure provides that the isobaric labeling reagent comprises an amine-, thiol-, or carbonyl-reactive moiety and an ionizable moiety comprising a stable heavy isotope. In some embodiments, the present disclosure provides that the isobaric labeling reagent comprises a tandem mass tag (TMT) reagent or an isobaric tag for relative and absolute quantitation (iTRAQ) reagent.

In some embodiments, the present disclosure provides that the glycan-linked isotopic tag is specific for a disaccharide linkage type of the enriched glycan-modified macromolecules, or a monosaccharide of the glycan of the enriched glycan-modified macromolecules.

In some embodiments, the present disclosure provides that the glycan-linked isotopic tag corresponds to a heavy isotope labeled derivative of a predetermined monosaccharide of a glycan attached to the glycan-modified macromolecules.

In some embodiments, the present disclosure provides that the glycan-linked isotopic tag comprises sialic acid or a derivative thereof, galactose or a derivative thereof, fucose or a derivative thereof, N-acetylglucosamine or a derivative thereof, N-acetylgalactosamine or a derivative thereof, mannose or a derivative thereof, glucose or a derivative thereof, or any combination thereof. In some embodiments, the present disclosure provides that the isotopic tag comprises sialic acid or a derivative thereof, wherein the sialic acid or a derivative thereof comprises N-Acetylneuraminic acid (Neu5Ac), N-Glycolylneuraminic acid (Neu5Gc), or 2-keto-3-deoxy-D-glycero-D-galacto-nononic acid (KDN).

In some embodiments, the present disclosure provides that the glycan-linked isotopic tag comprises a naturally occurring monosaccharide comprising one, two, three, four, five or six 13C or 16O atoms.

In some embodiments, the present disclosure provides that comprising digesting the biological sample with a protease prior to subjecting the biological sample to enriching for glycan-modified macromolecules step. In some embodiments, the present disclosure provides that further comprising subjecting the biological sample to a reverse-phase liquid chromatography.

In some embodiments, the present disclosure provides subjecting the biological sample to the reverse-phase liquid chromatography occurs before or substantially at the same time as subjecting the biological sample to the HILIC or lectin affinity chromatography. In some embodiments, the present disclosure provides that subjecting the biological sample to the biological sample to the HILIC or lectin affinity chromatography occurs before or substantially at the same time as subjecting the reverse-phase liquid chromatography. In some embodiments, the present disclosure provides that the biological sample is not dried and is not further reconstituted prior to subjecting to reverse-phase liquid chromatography or HILIC. In some embodiments, the present disclosure provides that the reverse phase liquid chromatography and HILIC is configured to be deposited in the same device. In some embodiments, the present disclosure provides that the reverse-phase liquid chromatography comprises a non-polar stationary phase selected from the group consisting of C-18, C-12, C-8, C-3, phenyl, biphenyl, and any combinations thereof.

In some embodiments, the present disclosure provides for subjecting the biological sample to a metal oxide layer. In some embodiments, the present disclosure provides for peptides, glycopeptides, and phosphopeptides. In some embodiments, the present disclosure provides that the HILIC comprises one or more materials selected from the group consisting of cellulose, unmodified silica, silica modified with diols, silica modified with cyanos, silica modified with aminos, silica modified with a zwitterionic sulfobetaine, silica modified with alkylamides, and any combinations thereof.

In some embodiments, the present disclosure provides for separating peptides from glycopeptides.

In some embodiments, the disclosure provides a method for using a classifier capable of distinguishing a subject with a disease state, comprising: adding the set of mass spectrometry standards disclosed herein to a biological sample from the subject to generate a mixed biological sample; subjecting the mixed biological sample to enrichment of PTM-modified macromolecules to generate an enriched mixed biological sample comprising PTM-modified macromolecules; performing a mass spectrometry assay on the enriched mixed sample to obtain a plurality of mass/charge ratios and intensities corresponding to PTM-modified macromolecules of the enriched mixed biological sample; and applying a trained machine learning classifier to the plurality of mass/charge ratios and intensities corresponding to glycan-modified macromolecules of the enriched mixed biological sample to obtain an output classification of whether the biological sample from the subject is associated with the disease state.

In some embodiments, the present disclosure provides that the mass spectrometry assay is an LC/MS/MS assay.

In some embodiments, the present disclosure provides that the classifier is selected from the group consisting of an artificial neural network, a support vector machine, a linear model, a non-linear model, a parametric model, a non-parametric model, a Bayesian model, a gaussian process, a binary classifier, a multilabel classifier, a non-binary classifier, a deep neural network, an ensemble method, a tree based model, a clustering method, a Markov model, and a combination thereof. In some embodiments, the present disclosure provides that the classifier comprises a linear model. In some embodiments, the present disclosure provides that the linear model comprises a ridge classifier, a stochastic gradient classifier, a passive aggressive classifier, or a perceptron. In some embodiments, the present disclosure provides that the classifier comprises a non-linear model. In some embodiments, the present disclosure provides that the non-linear model comprises a logistic regression model, a naïve bayes method, a kernel support vector machine, or a k nearest neighbor method. In some embodiments, the present disclosure provides that the classifier comprises an ensemble model. In some embodiments, the present disclosure provides that the ensemble model comprises a forest method, a random forest method, an extra trees classifier, an adaboost method, gradient boost method, or a voting classifier. In some embodiments, the present disclosure provides that the classifier comprises an artificial neural network. In some embodiments, the present disclosure provides that the classifier comprises a deep neural network model. In some embodiments, the present disclosure provides that the deep neural network is trained in an unsupervised setting, a supervised setting, a semi-supervised setting, or a self-supervised setting. In some embodiments, the present disclosure provides that the classifier uses a dimension reduction analysis. In some embodiments, the present disclosure provides that the dimension reduction analysis is selected from the group consisting of a principal component analysis, an independent component analysis, a linear discriminant analysis, a non-negative matrix factorization, a truncated singular value decomposition, a variational autoencoder, a transformer model, a u-net, a generative adversarial network, and any combination thereof.

In some embodiments, the present disclosure provides that the PTM-modified macromolecule comprises a macromolecule modified by glycosylation, ubiquitination, phosphorylation, acetylation, or any combination thereof. In some embodiments, the PTM-modified macromolecule is a glycan-modified macromolecule.

Disclosed herein, in some aspects, are methods comprising: combining a biological sample with a double-labeled peptide standard comprising a peptide comprising a first label, and comprising a post-translational modification (PTM) comprising a second label; and identifying or measuring, based on the double-labeled peptide standard, an endogenous protein of the biological sample, wherein the endogenous protein comprises the PTM. In some aspects, the PTM comprises a glycan, ubiquitin, phosphate, or acetyl. In some aspects, the endogenous protein comprises a glycoprotein. In some aspects, the biological sample comprises a biofluid. In some aspects, the biofluid comprises blood, serum, or plasma. In some aspects, the biological sample is obtained from a subject. In some aspects, the subject is a human. Some aspects including comprising enriching or purifying the biological sample for proteins comprising the PTM prior to combining the biological sample with the double-labeled peptide standard. In some aspects, enriching or purifying the biological sample for peptides comprising the PTM comprises performing chromatography. In some aspects, the chromatography comprises hydrophilic interaction liquid chromatography (HILIC), liquid chromatography, solid-phase chromatography, column chromatography, affinity chromatography, ion exchange chromatography, or size exclusion chromatography, or a combination thereof. In some aspects, the first label comprises an offset label. In some aspects, the offset label comprises the same chemical structure as the isobaric tags for endogenous samples. In some aspects, the offset label has higher molecule weight by incorporating more heavy isotope elements than isobaric tags for endogenous samples. In some aspects, the endogenous protein of the biological sample comprises the first isobaric label. In some aspects, the biological sample is combined with additional biological samples. In some aspects, the additional biological samples comprises additional isobaric tags and the endogenous protein. In some aspects, identifying or measuring the endogenous protein comprises performing a multiplex measurement of the endogenous protein in the biological sample combined with the additional biological samples. In some aspects, the offset label comprises a similar chemical structure as the first isobaric tag on the endogenous protein of the biological sample. In some aspects, the offset label comprises a higher molecular mass than the first isobaric tag on the endogenous protein of the biological sample. In some aspects, the first isobaric tag identifies the biological sample in the multiplex measurement, and wherein the additional isobaric tags identify the additional biological samples in the multiplex measurement. In some aspects, the second label comprises an isotope label. In some aspects, the second label is added to the double-labeled peptide standard by enzymes. Some aspects include comprising generating the double-labeled peptide standard. In some aspects, generating the double-labeled peptide standard comprises adding the offset label to the biological sample. In some aspects, generating the double-labeled peptide standard comprises adding the first label and the second label to a portion of the biological sample. In some aspects, generating the double-labeled peptide standard comprises pooling the portion of the biological sample and the additional biological samples for enriching or purifying glycopeptides. In some aspects, identifying or measuring the endogenous protein of the biological sample comprises performing mass spectrometry. In some aspects, identifying or measuring, based on the double-labeled peptide standard, the endogenous protein of the biological sample comprises comparing a mass spectrum of the endogenous peptide to a mass spectrum of the double-labeled peptide standard. Some aspects include comprising contacting the biological sample with a particle to capture the endogenous protein on the particle. In some aspects, the particle comprises a nanoparticle. Some aspects include comprising adding the endogenous protein comprising the PTM to a mass spectrometry library. Some aspects include comprising obtaining a measurement of the endogenous protein. In some aspects, obtaining the measurement of the endogenous protein comprises measuring a mass spectrum of the endogenous protein. Some aspects include comprising inputting the measurement of the endogenous protein into a classifier to evaluate a biological state. In some aspects, the biological state comprises a healthy state. In some aspects, the biological state comprises a disease. In some aspects, the disease comprises a cancer. In some aspects, the cancer comprises pancreatic cancer. In some aspects, the cancer comprises lung cancer, breast cancer, colon cancer, liver cancer, or ovarian cancer.

Disclosed herein, in some aspects, are methods comprising: contacting a biological sample comprising with a particle to adsorb an endogenous protein of the biological sample to the particle, wherein the endogenous protein comprises a post-translational modification (PTM); and combining the adsorbed endogenous protein with a double labeled peptide standard comprising an isotope labeled amino acid residue on the peptide and a labeled version of the PTM. Some aspects include comprising identifying or measuring the endogenous protein based on the double-labeled peptide standard.

Disclosed herein, in some aspects, are methods comprising: combining a biological sample with a double-labeled lipid standard comprising a lipid comprising a first label, and comprising a glycan comprising a second label; and identifying or measuring, based on the double-labeled lipid standard, an endogenous lipid of the biological sample, wherein the endogenous lipid comprises the glycan. In some aspects, the endogenous lipid comprises a glycolipid. In some aspects, the biological sample comprises a biofluid. In some aspects, the biofluid comprises blood, serum, plasma, urine, tears, semen, milk, vaginal fluid, mucus, saliva, or sweat, or combinations thereof. In some aspects, the biological sample is obtained from a subject. In some aspects, the subject is a human. Some aspects include comprising enriching or purifying the biological sample for lipids comprising the glycan prior to generating the double-labeled lipid standard. In some aspects, enriching or purifying the biological sample for lipids comprising the glycan comprises performing chromatography. In some aspects, the chromatography comprises hydrophilic interaction liquid chromatography (HILIC), liquid chromatography, solid-phase chromatography, column chromatography, affinity chromatography, ion exchange chromatography, or size exclusion chromatography, or combinations thereof. In some aspects, the first label comprises an offset label. In some aspects, the offset label comprises an isotope label. In some aspects, the second label comprises an isotope label. In some aspects, the second label is added to the double-labeled lipid standard by enzymes or by organic synthesis. Some aspects include comprising generating the double-labeled lipid standard. In some aspects, generating the double-labeled lipid standard comprises adding the first label to a portion of the biological sample. In some aspects, generating the double-labeled lipid standard comprises adding the first label and the second label to the portion of the biological sample. In some aspects, generating the double-labeled lipid standard comprises pooling the portion of the biological sample and additional biological samples for enriching or purifying glycolipids. In some aspects, identifying or measuring, based on the double-labeled lipid standard, the endogenous lipid of the biological sample comprises performing mass spectrometry. In some aspects, identifying or measuring, based on the double-labeled lipid standard, the endogenous lipid of the biological sample comprises comparing a mass spectrum of the endogenous lipid to a mass spectrum of the double-labeled lipid standard. Some aspects include comprising contacting the biological sample with a particle to capture the endogenous lipid on the particle. In some aspects, the particle comprises a nanoparticle. Some aspects include comprising adding the endogenous lipid comprising glycans to a mass spectrometry library. Some aspects include comprising obtaining a measurement of the endogenous lipid. In some aspects, obtaining the measurement of the endogenous lipid comprises measuring a mass spectrum of the endogenous lipid. Some aspects include further comprising inputting the measurement of the endogenous lipid into a classifier to evaluate a biological state. In some aspects, the biological state comprises a healthy state. In some aspects, the biological state comprises a disease. In some aspects, the disease comprises a cancer. In some aspects, the cancer comprises pancreatic cancer. In some aspects, the cancer comprises lung cancer, breast cancer, colon cancer, liver cancer, or ovarian cancer.

Disclosed herein, in some aspects, are methods comprising: combining a biological sample with a double-labeled biomolecule standard comprising a biomolecule comprising a first label on a first portion of the biomolecule, and comprising a second label on a second portion of the biomolecule; and identifying or measuring, based on the double-labeled biomolecule standard, an endogenous biomolecule of the biological sample, wherein the endogenous biomolecule comprises the first label on the first portion of the biomolecule or the second label on the second portion of the biomolecule. In some aspects, the biomolecule comprises a peptide or a lipid.

Glycoproteomics may be used to study protein glycosylation. Due to the low abundance of glycopeptides (peptides that are glycosylated), it is useful to enrich glycopeptides from non-glycopeptides for in-depth analyses. To resolve the need of a more efficient and easier method for isolating and enriching glycopeptides, the present disclosure provides for an array of systems and methods to achieve that conclusion. Rather than using multiple devices in order to achieve this end product, instead this disclosure includes methods and devices that allow for a useful product to be reached with a single device. With fewer steps and less transfer of material, the loss of product that other methods and devices encounter is reduced or not present in methods and devices of this disclosure. As a result, the device and methods provided herein allow for a simpler overall process for enriching glycopeptides with a higher specificity and peptide structure match.

Various aspects of the present disclosure provide a device for enriching glycopeptides. The device may comprise a device body defining an interior volume comprising a distal opening and a proximal opening and chromatography material in the interior volume. The chromatography material may comprise a distal layer of stationary reverse phase chromatography material, a proximal layer of stationary reverse phase chromatography material, and a layer of hydrophilic interaction chromatography materials between the distal and proximal layers of reverse phase chromatography material. Each of the layers of chromatography materials comprises a homogenous composition, and wherein a liquid is able to flow through the volume in either direction.

The device body may comprise a pipette tip, a pipette, or a chromatography column.

In various aspects of the device, the interior volume of the device body may be loaded with a liquid from the distal opening or the proximal opening. Flowing a liquid through the chromatography material, from either the proximal to the distal end or vice versa, may enrich the glycopeptides of the liquid or may remove a variety of non-glycopeptide material from the liquid.

Various aspects of the present disclosure provide chromatography materials that may be used in the device. The stationary reverse phase chromatography material may comprise surface modified silica. The surface modified silica may be, in some cases, modified with an unmodified alkyl ligand such as C18, C8, C4 or by a modified alkyl ligand. The hydrophilic interaction chromatography material may comprise unmodified silica. Alternatively, the hydrophilic interaction chromatography material may comprise silica modified with a functional group. The functional group may be chosen from a diol, cyano, amino, alkylamide, or a combination thereof. Alternatively, the functional group may comprise a zwitterionic sulfbetaine. Alternatively, the hydrophilic interaction chromatography materials may be cellulose. The cellulose may be a particular polymorph, density, or size. The cellulose may be microcrystalline cellulose.

Aspects of the present disclosure provide for a method and device that may be, in some cases, used with various fluids. There may be liquid flowing over the chromatography material. The liquid may be a biological sample or derived from a biological sample.

Various aspects of the present disclosure provide for a device that may use chromatography material organic phase chromatography solvent and aqueous phase chromatography solvent.

Various aspects of the present disclosure provide for a device or method that may be a size and configuration suitable for large scale production of glycopeptides. The device or method may be integrated into an automated platform.

Various aspects of the present disclosure provide for a kit containing the device of the present disclosure. The kit may provide the device of the disclosure in a fully assembled state. Alternatively, the kit may comprise the individual components of the device in a form suitable for user assembly. The kit may further comprise instructions on the assembly and/or use of the device.

Various aspects of the present disclosure provide for a method for enriching glycopeptides. This method may comprise applying a biofluid comprising the glycopeptides onto a first stationary reverse phase chromatography material, washing the first stationary reverse phase chromatography material using an aqueous mobile phase, eluting the glycopeptides from the first stationary reverse phase chromatography material and applying it onto a distinct hydrophilic interaction chromatography material using an organic mobile phase, eluting the glycopeptides from the distinct hydrophilic interaction chromatography material and applying it onto a distinct second stationary reverse phase chromatography material using an aqueous mobile phase, eluting the purified or enriched glycopeptides from the distinct second stationary reverse phase chromatography material using an organic mobile phase. The chromatography materials may each comprise a homogenous composition.

Various aspects of the present disclosure provide for a device for enriching glycopeptides. The device may include a polar stationary phase; a non-polar stationary phase; and one or more mobile phases; wherein the one or more mobile phases is capable of passing through the polar stationary phase and the non-polar stationary phase. The device of the present disclosure may further comprise a container that is configured to contain the polar stationary phase and the non-polar stationary phase. The polar stationary phase may be above the non-polar stationary phase. The polar stationary phase is Hydrophilic Interaction Liquid Chromatography (HILIC) material. The non-polar stationary phase may be reverse phase chromatography material. The non-polar stationary phase may be C18. The one or more mobile phases includes a mobile phase, wherein the mobile phase includes at least one acid and at least one polar solvent. The one or more mobile phases includes a mobile phase, wherein the mobile phase includes at least one acid and water. The polar stationary phase, the non-polar stationary phase, and the mobile phases may be as described elsewhere herein.

Various aspects of the present disclosure provide for a method for enriching glycopeptides as shown in FIG. 25. This method may comprise applying a biofluid comprising the glycopeptides onto a hydrophilic interaction chromatography material. This method may comprise drying biofluids. This method may comprise reconstituting biofluids. The biofluids may be reconstituted using an organic mobile phase (e.g., about 85% acetonitrile (ACN) with about 1% trifluoroacetic acid (TFA)) before applying the biofluid to the hydrophilic interaction chromatography material. After applying the biofluid to the hydrophilic interaction chromatography material, the method may include washing the hydrophilic interaction chromatography material with an organic mobile phase (e.g., about 85% ACN/1% TFA). Any bound glycopeptides may be eluted by an aqueous mobile phase (e.g., about 0.1% formic acid).

Various aspects of the present disclosure provide for a method for enriching glycopeptides as shown in FIG. 26. This method may comprise applying a biofluid comprising the glycopeptides onto a stationary reverse phase chromatography material (e.g., C18) and then onto a hydrophilic interaction chromatography material. The two stationary materials may be deposited in separate devices, and the separated devices may be connected fluidly with each other. In other cases, the two stationary materials may be deposited in the same device with two separate stationary materials. The method may include washing the stationary reverse phase chromatography material with an organic mobile phase (e.g., about 85% ACN/1% TFA). The method may include equilibrating the stationary reverse phase chromatography material with an aqueous mobile phase (e.g., about 1% TFA). The method may include washing the stationary reverse phase chromatography material with an aqueous mobile phase (e.g., about 1% TFA). The method may include washing the stationary reverse phase chromatography material with an organic mobile phase (e.g., about 85% ACN/1% TFA) to elute any bound peptides. The eluted bound peptides may be loaded onto the hydrophilic interaction chromatography material. The method may include priming the hydrophilic interaction chromatography material before the washing step. The priming of the hydrophilic interaction chromatography material may be done by equilibrating with an organic mobile phase (e.g., about 85% ACN/1% TFA). The hydrophilic interaction chromatography material may be washed with an organic mobile phase (e.g., about 85% ACN/1% TFA). Any bound glycopeptides may then be eluted using an aqueous mobile phase (e.g., about 0.1% formic acid) after the elution of peptides that are not modified by glycans.

Various aspects of the present disclosure provide for a method for enriching glycopeptides using a single device as shown in FIG. 27. This method may comprise applying a biofluid (e.g., digest) comprising the glycopeptides to a single device, wherein the single device comprises a hydrophilic interaction chromatography material and a stationary reverse phase chromatography material. The method may include stacking the hydrophilic interaction chromatography material onto the stationary reverse phase chromatography material. Stacking the hydrophilic interaction chromatography material on the stationary reverse phase chromatography material within the device may be done such that a liquid (e.g., mobile phases) passes through the hydrophilic interaction chromatography material before the stationary reverse phase chromatography material (or vice versa). The method may include using an organic mobile phase. The method may include using an aqueous mobile phase. The biofluid may comprise glycopeptides and other peptides (wherein the other peptides are different from the glycopeptides). The method may include using an organic mobile phase to remove the other peptides from the device. The method may include using an organic mobile phase to separate the other peptides from the glycopeptides. The method may include using an aqueous mobile phase to remove the glycopeptides from the device. The method may include washing the two materials of the device with an organic mobile phase (e.g., about 85% ACN/1% TFA). The method may include equilibrating the two materials of the device with an aqueous mobile phase (e.g., about 1% TFA). The biofluid may be acidified. The biofluid may be acidified using 1% TFA. The method may include washing the device after adding the biofluid. The method may include washing the device after adding the biofluid using an aqueous mobile phase (e.g., 1% TFA). The method may include washing the device after adding the biofluid using an aqueous mobile phase (e.g., 1% TFA) more than once (e.g., twice). The peptides from the biofluid may then eluted from the device using an organic mobile phase (e.g., about 85% ACN/1% TFA). The glycopeptides from the biofluid may then be eluted from the device using an aqueous mobile phase (e.g., about 2% ACN/0.1% formic acid). The chromatography materials may each comprise a homogenous composition.

In some aspects of the present disclosure, peptides may be enriched. The biofluid may further comprise non-glycosylated peptides. These non-glycosylated peptides may be eluted from the device after a first organic mobile phase is applied to the device. The peptide may be eluted in a solution that comprises a different composition than the initial biofluid.

This method may take place entirely within a single device. The device may comprise a device body that defines an interior volume comprising a distal opening and a proximal opening.

Various aspects of the present disclosure provide for chromatography materials that may be used in the disclosed method. The stationary reverse phase chromatography material may comprise surface modified silica. The surface modified silica is modified with an unmodified alkyl ligand. Alternatively, the surface modified silica may be modified by an alkyl ligand.

The hydrophilic interaction chromatography materials may comprise unmodified silica. Alternatively, the hydrophilic interaction chromatography materials may be modified silica modified with a functional group. The functional groups may comprise diols, cyanos, aminos, alkylamide, or a mixture thereof. Alternatively, the functional group may comprise a zwitterionic sulfbetaine. Alternatively, the hydrophilic interaction chromatography materials may be cellulose. The cellulose may be a particular polymorph, density, or size. It may be microcrystalline cellulose.

Various aspects of the present disclosure provide for a method that may, in some cases, be used with a biofluid. The biofluid may be a biological sample or may be derived from a biological sample. In other cases, the biofluid may be a pooled biological sample collected from more than one subject. The subject may be a human. The biofluid may be modified prior to being used in the method of the present disclosure, such as the proteins contained in the biofluid have been digested with various enzymes or other conventional methods. It may comprise salts, buffers, proteins, peptides, protein digests, and nucleic acids among other chemicals and biological molecules.

In various aspects of the present disclosure various liquids may be used. The organic mobile phase of the disclosed method may be acetonitrile, methanol, or tetrahydrofuran among others. The organic mobile phase may comprise water. The organic mobile phase may comprise trifluoracetic acid. The aqueous mobile phase of the disclosed method may vary in composition. This aqueous mobile phase may be water. The aqueous mobile phase may contain a buffer system. The buffer system may contain trifluoroacetic acid, phosphate, formic acid, or acetic acid among other chemicals.

Various aspects of the present disclosure provide that the method may be implemented in a variety of situations. The method may provide for the large-scale production of purified glycopeptides. The method may integrate into an automated platform.

Various aspects of the present disclosure provide for changing the composition of the biofluid. The enriched glycopeptide may comprise a buffer that is different than the buffer of the biofluid used in the method. This difference may be the addition or removal of the various components that comprise the buffers. The enriched glycopeptide may comprise a lower concentration of salts than the biofluid used in the method.

Various aspects of the present disclosure provide for the enrichment of other components of the biofluid. The eluted material from some of the steps of the method may comprise peptides. The peptides may be enriched or may be eluted in a fluid that is comprised of different chemicals and biomolecules than the biofluid used in the method.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 is a flowchart depicting an example workflow analysis disclosed herein.

FIG. 2 illustrates an example of a double-labeled peptide structure in a blood, serum, or plasma sample comprising a labeled peptide backbone and a labeled structure-specific glycan portion.

FIGS. 3A, 3B, and 3C illustrate images of example glycan structures. FIG. 3A shows one sialic acid with a corresponding residual monoisotopic mass. FIG. 3A also shows two fucoses with a corresponding residual monoisotopic mass. FIG. 3B shows a common glycan structure in blood, serum, or plasma with a corresponding monoisotopic mass. FIG. 3C shows a CA-19-9 containing structure with a corresponding isotopic mass.

FIG. 4 illustrates an example of a graph depicting the mass differences of the structures in FIG. 3B and FIG. 3C before and after backbone labeling and glycan labeling.

FIG. 5 is a flowchart depicting an example workflow analysis disclosed herein.

FIG. 6 illustrates an example of a double-labeled glycopeptide standard and a list of monosaccharide residues that may be isotope labeled.

FIG. 7 illustrates an example of a double-labeled glycolipid standard and a list of monosaccharide residues that may be isotope labeled.

FIG. 8 is a flowchart depicting an example workflow analysis disclosed herein.

FIG. 9 shows a workflow from sample to data analysis to in-depth analysis on the glycoproteomics platform.

FIG. 10A shows a workflow comparison of a hybrid CID/EAD and a CID only approach for glycopeptide discovery.

FIG. 10B shows a comparison of the hybrid and CID platforms for glycoPSMs and Unique glycopeptide IDs.

FIG. 10C shows a Venn diagram of unique glycopeptide IDs for hybrid and CID only methods.

FIG. 11 shows PSM and glycopeptide IDs from three mass spectrometry methods.

FIG. 12A shows a Venn diagram comparing glycopeptides identified with and without nanoBooster.

FIG. 12B shows the average size of glycan identified with and without nanoBooster.

FIG. 13A shows a heatmap of m z over retention time.

FIG. 13B shows an extracted ion mobilogram (EIM).

FIG. 14A shows a Venn diagram of unique glycopeptides identified by timsTOF Pro2 with and without nanobooster and ZenoTOF with CID and hybrid acquisitions.

FIG. 14B shows a Venn diagram of glycopeptide backbone identified by timsTOF Pro2 and ZenoTOF.

FIG. 14C shows a Venn diagram of glycopeptide backbone identified by timsTOF Pro2 and ZenoTOF without EAD.

FIG. 15 shows an example mass spectrum (e.g., MS1 spectra) for synthetic peptides labeled as described in Example 6.

FIG. 16 shows an example mass spectrum (e.g., MS2 spectra) for synthetic peptides labeled as described in Example 6.

FIG. 17 shows an example chromatogram (top) and corresponding spectra (e.g., MS2 spectra) for synthetic peptides labeled as described in Example 6.

FIG. 18 shows a MS1 spectra of three species of a natural fetuin-derived glycopeptide labeled with TMT0, TMT6, and TMT6+N4H5S*2 labels as described in Example 6.

FIG. 19 shows an example mass spectrum (e.g., MS2 spectra) for three species of a natural fetuin-derived glycopeptide labeled with TMT0, TMT6, and TMT6+N4H5S*2 labels as described in Example 6.

FIG. 20A shows example structures of TMT mass tags and isobaric labeling reagents usable with methods according to the disclosure.

FIG. 20B shows example TMT reporter ions for the mass tags depicted in FIG. 20A.

FIG. 21 shows example structures of DiLeu mass tags and isobaric labeling reagents usable with methods according to the disclosure.

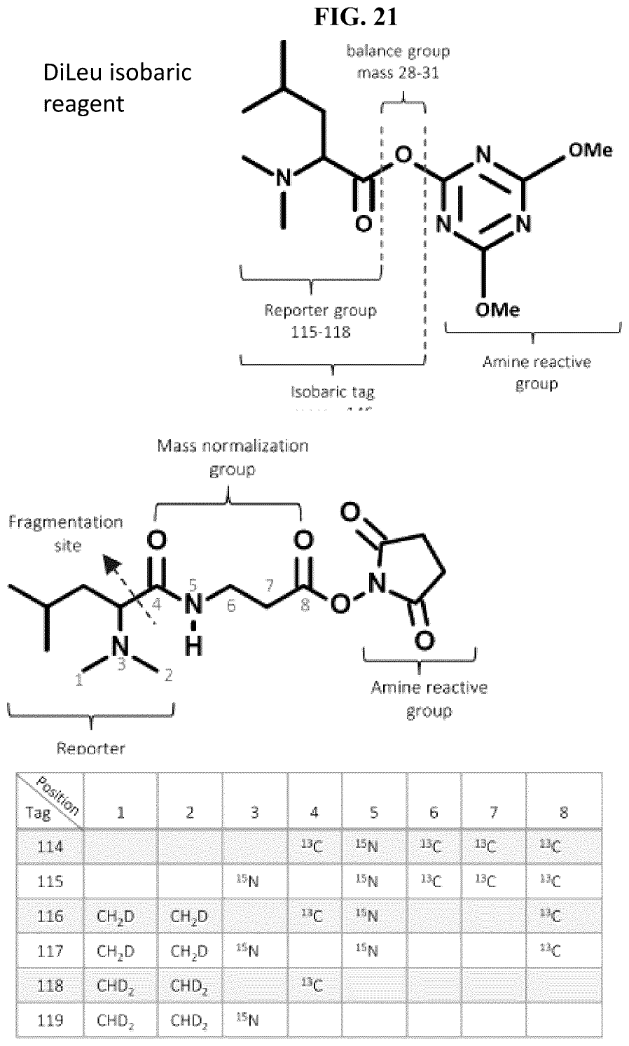

FIG. 22 shows example structures of iTRAQ/TMT mass tags and isobaric labeling reagents usable with methods according to the disclosure that include carbonyl-reactive groups.

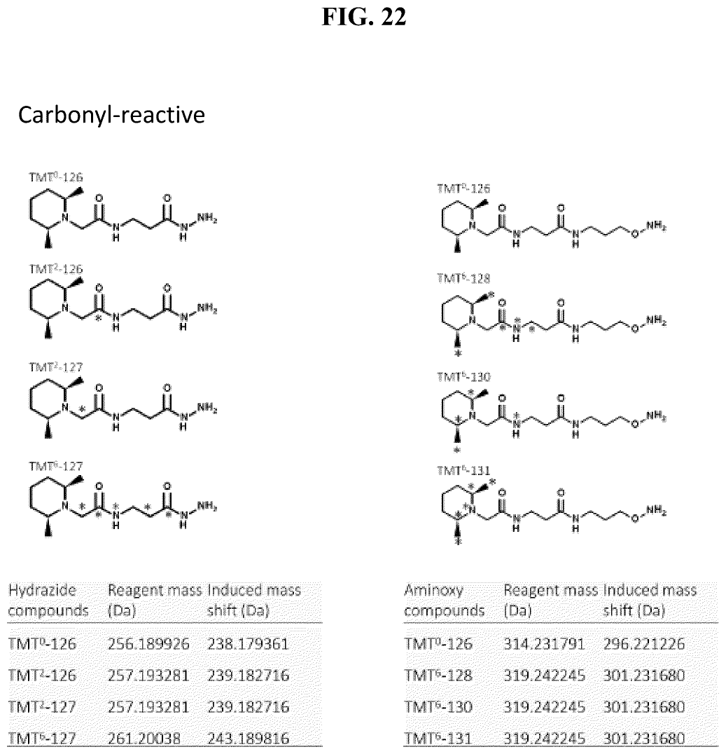

FIG. 23 is a diagram of a stepwise method for enrichment of glycopeptides using an enrichment device.

FIG. 24 is a diagram showing assembly of an enrichment device with a pipette tip as an example device body.

FIG. 25 depicts Workflow 1.

FIG. 26 depicts Workflow 2.

FIG. 27 depicts one device method.

FIG. 28 shows coefficient variation values of a sample digest and a peptide fraction following workflow 2.

FIG. 29 shows a Venn diagram of overlapping peptide precursor identification following proteomic analysis comparing a peptide fraction of workflow 2 and a sample digest.

FIG. 30 depicts a linear correlation of peptide precursor identification between a peptide fraction of workflow 2 and a sample digest.

FIG. 31 shows a comparison of the number of glycopeptide peptide spectrum match (PSM) and number of unique glycopeptide identifications following glycoproteomic analysis for workflow 1 and workflow 2.

FIG. 32 shows a Venn diagram of the unique glycopeptide identifications following workflow 1 and workflow 2.

FIG. 33 shows a MS2 spectra (GlycoMS2/Total MS2) depicting glycopeptide specificity for workflow 1 and workflow 2.

FIG. 34 shows coefficient variation values of a sample digest and a peptide fraction following a one device method.

FIG. 35 shows a Venn diagram of overlapping peptide precursor identifications following proteomic analysis comparing a peptide fraction of one device and a sample digest.

FIG. 36 shows a linear correlation of peptide precursor identification between a peptide fraction of one device and a sample digest.

FIG. 37 shows a comparison of the number of glycopeptide peptide spectrum matches (PSM) and number of unique glycopeptide identifications for workflow 1 and one device.

FIG. 38 shows a Venn diagram of overlapping glycopeptide precursor identifications comparing a glycopeptide fraction of one device and a glycopeptide fraction of workflow 1.

FIG. 39 shows MS2 spectra (GlycoMS2/Total MS2) depicting glycopeptide specificity for workflow 1 and one device.

FIG. 40 shows an enrichment device with metal oxide layer, non-polar stationary phase, and polar stationary phase and steps for the enrichment and/or separation of peptides, glycopeptides, and phosphopeptides.

FIG. 41 shows an embodiment of a system that is programmed or otherwise configured to implement methods provided herein.

### DETAILED DESCRIPTION

### Overview

Post-translational modifications (PTMs) represent an important level of post-translational peptide activity control, as post-translational modifications to peptides can alter localization, activity, association with accessory factors, trafficking, and turnover of associated peptides. As aberrations in localization, activity, association with accessory factors, trafficking, and turnover of peptides (e.g., metabolic enzymes, or disease-causative peptides) can be associated with cellular pathophysiology of disease, investigation of disease-specific PTMs represents an important avenue of research for disease identification and intervention. As PTMs are associated with changes in mass of the parent peptide, mass spectrometry techniques represent one of the most versatile routes of PTM detection and characterization. Types of PTMs include, but not limited to, glycosylation, ubiquitination, phosphorylation, acetylation, or a combination thereof. Aberrant changes to proteins by one or more PTMs can lead to cellular stress, cellular malfunction, abnormal cellular proliferation, or abnormal growth factor signaling, which have been linked to various human diseases (e.g., cancer). Thus, PTMs may be used for prognosis, detection, and monitoring of human diseases.

Glycosylation is an important PTM that regulates diverse biological functions. Types of glycosylation include N-linked glycosylation, O-glycosylation, mucin-type O-glycosylation, and glycosphingolipid glycosylation. Glycoproteins, composed of glycans, present in serum may serve as important biomarkers for disease or cancer detection. Non-limiting examples of glycans associated with cancer include proteoglycans, glycosaminoglycans, glycosylphosphatidylinositol-anchored glycoproteins, CD43, CD45, galectins, siglecs (e.g., CD22, CD169), selectin (e.g., E-selectin, P-selectin, and L-selectin), sialylated lewis antigens (e.g., Sialyl LeA, Sialyl LeX, CA19-9 (which is a molecular that contains sialyl LeA)), core-fucosylated glycans, globo H, bisecting GlcNAc, truncated O-glycans (e.g., Thomsen-nouvelle antigen (Tn), Sialyl Tn, T), Sda/CAD antigens, Neu5Gc-glycans, gangliosides (e.g., disialoganglioside 1 (GD1), monosialic ganglioside 2 (GM2), CD2, CD3, N-acetyl-GD2), alpha2,6-linked sialic acid in N-glycans, N-glycan branching or variant thereof. Some glycoproteins are FDA-approved protein-based cancer biomarkers. For example, CA 125 antigen can be used for ovarian cancer detection, CA 19-9 can be used for pancreatic cancer detection, and CA 15-3 can be used for breast cancer detection. While specific glycoproteins have been identified as potential biomarkers, there are still challenges to identifying the different glycoproteins for disease or cancer detection. For one, it is difficult to identify different glycoforms or glycan isoforms due to glycosidic linkages. Standard glycoproteomics analysis with liquid chromatography-mass spectrometry (LC-MS), for example, cannot readily provide linkage information, thereby requiring a need for improvement in glycoproteomics.

Ubiquitination, phosphorylation, and acetylation are additional examples of PTMs that may be used as biomarkers for disease or cancer detection. Ubiquitination, phosphorylation, and acetylation may affect protein charge, conformation, stability, synthesis, localization, or interaction with other molecule, further affecting protein function, signaling pathways, and various cellular processes.

Ubiquitination occurs when ubiquitin is attached to a substrate by a three-step enzymatic cascade involving ubiquitin-activating enzyme (E1), ubiquitin-conjugating enzyme (E2), and ubiquitin-protein ligase (E3). Modification of a protein may occur as a single ubiquitin on a single lysine (monoubiquitin), a single ubiquitin on multiple lysines (multiubiquitin), or as ubiquitinated chains in which lysines on the ubiquitin molecule are further modified through ubiquitination (polyubiquitin). In some cases, ubiquitin itself may be modified by other PTMs (e.g., phosphorylation or acetylation on ubiquitin). Non-limiting examples of ubiquitination associated with disease include MYC protein ubiquitination, FBP-1 protein ubiquitination, or p53 ubiquitination.

Protein phosphorylation is catalyzed by protein kinase to transfer the gamma-position phosphate group of ATP or GTP to amino acid residues of a substrate protein. Phosphorylated protein may be O-phosphate protein, N-phosphate protein, acyl phosphate protein, and S-phosphate protein. In addition, non-limiting examples of events of phosphorylation associated with disease include phosphorylation sites downstream of Cdk5, STAT3 phosphorylation, prohibitin (PHB) phosphorylation, Thr/Ser kinase phosphorylation, or SIRT1 phosphorylation.

Protein acetylation occurs when the acetyl group from acetyl coenzyme A is introduced to a specific site on a polypeptide chain. Mechanisms for acetylation may be lysine acetylation, N-terminal protein acetylation, and O-acetylation. In addition, non-limiting examples of acetylation associated with disease include SOD2 acetylation, p53 acetylation, acetylation of H3K27, histone acetylation of H3K3me3 or acetylation of H4 (e.g., at lysine (K)16).

However, despite the fact that glycosylation is biologically important and of interest for disease identification and intervention, analysis of glycosylation by mass spectrometry can involve a number of analytical challenges. A first challenge relates to sensitivity—as the total cellular abundance of glycopeptides (or residency of peptide glycosylation events) can be low, enrichment methods that separate glycoproteins from their non-modified precursors are often necessary. A second challenge relates to the heterogeneity and degeneracy of glycosylation—as glycan chains can be diverse in composition but represent a large number of structural isomers (e.g., linkage isomers, positional isomers, and functional isomers), obtaining biologically-relevant data can involve the need to distinguish unique glycan species that are difficult to resolve by mass. A third challenge relates to the distinct chemical nature of glycans from peptides—as glycans are composed of chemically distinct monomers and fragment under mass spectrometric methods (e.g., collision induced dissociation—CID, and electron activated dissociation—EAD) differently compared to peptides, and search algorithms that reliably identify peptides by their fragmentation patterns are not applicable to analysis of complex glycans.

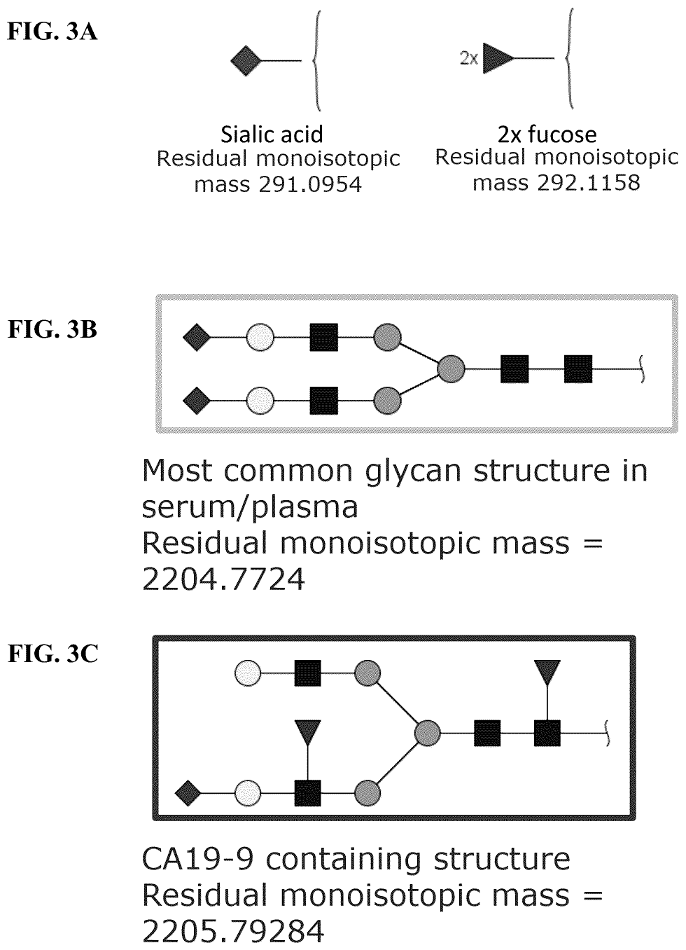

The heterogeneity in glycoproteins (e.g., the presence of a peptide with many different glycan structures attached to it in a sample) can represent a specific challenge in measuring or identifying all potential glycopeptide species even when the species to be resolved are not isomers of each other. An illustrative example is shown in FIGS. 3A-3C, which illustrate examples of both monosaccharides and linked glycans that are difficult to resolve. FIG. 3A shows that a glycopeptide structure comprising one sialic acid (residual monoisotopic mass of 291.0954 Dalton (Da)) differs only by 1.0204 Da when compared to a glycopeptide structure comprising two fucose (residual monoisotopic mass of 292.1158 Da), indicating that a mono sialic acid modification is difficult to distinguish from a double fucose modification. FIG. 3B and FIG. 3C compare two common complex saccharide modifications (one di-sialylated biantennary complex type N-glycan and one biantennary complex type N-glycan with core-fucosylation and terminal sialyl Lewis A, respectively) which, while they comprise unique repertoires of monosaccharides, nonetheless are similar in monoisotopic mass (e.g., 2204.7724 vs 2205.7928), indicating that even complex saccharides of different composition can be difficult to distinguish in mass spectrometry. Described herein are workflows that may address challenges distinguishing modifications such as these.

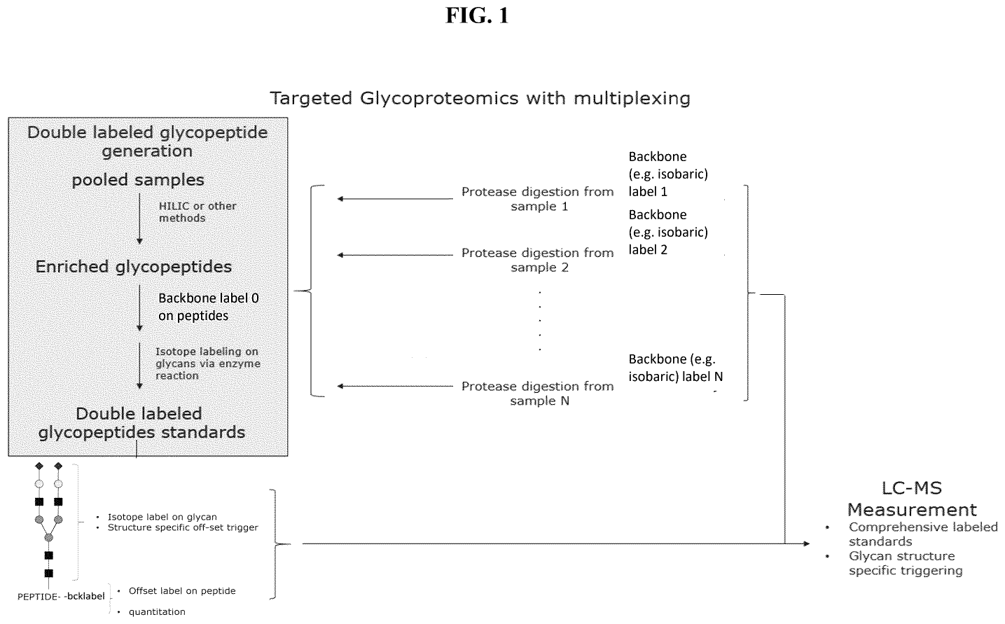

FIG. 1 depicts an example workflow strategy for glycopeptide analysis (which is optionally used to multiplex multiple distinct samples) that at least partially addresses the aforementioned challenges. In some embodiments, this strategy involves: (a) an optional glycopeptide enrichment step (e.g., HILIC purification or purification methods and devices disclosed herein); (b) creation of internal glycopeptide standards with mass labels linked to peptide- and glycan-specific locations in the glycopeptide; and (c) pooling of the internal glycopeptide standards with glycopeptides that are labeled with only an isobaric peptide-backbone label prior to mass spectrometry analysis (LC/MS/MS).

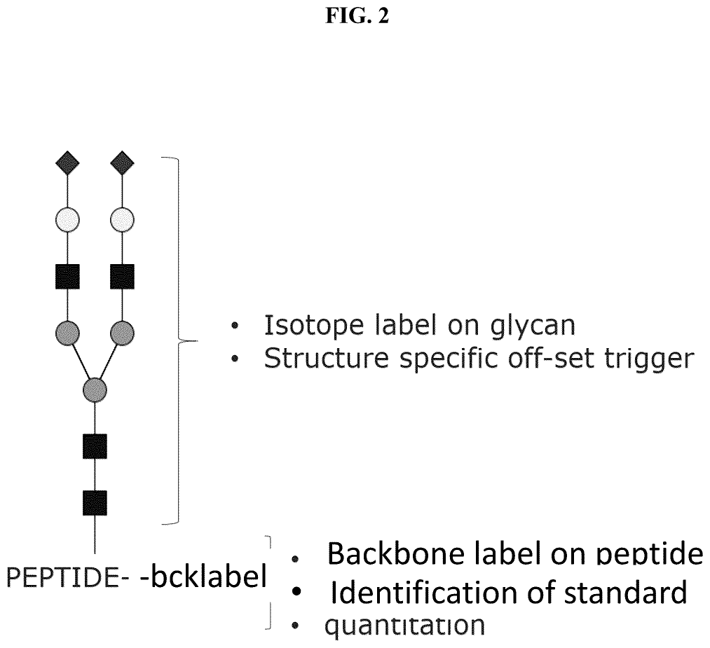

The internal glycopeptide standards can comprise structures according to FIG. 2 and can be generated via addition of both a peptide backbone-linked mass label (e.g., an isobaric tag, or any other chemical moiety with a characteristic mass) and a glycan-linked mass label (e.g., an isotopically labeled monosaccharide) to peptides from samples (e.g., endoprotease-generated peptides) (see e.g., FIG. 1). The peptide backbone mass label can be introduced via suitable peptide-targeting chemistry (e.g., amine-, carbonyl-, orthiol-reactive chemistry) and can provide for identification of the glycopeptide standards when spiked into an unlabeled sample (or a sample with glycopeptides that are labeled with only an isobaric peptide-backbone label, as in FIG. 1) as well serve as a relative reference for quantitating unlabeled glycopeptides (or glycopeptides that are labeled with only an isobaric peptide-backbone label) included in the same run (e.g., when the standards are spiked into an unlabeled sample). The glycan-linked mass label (which can be an isotopic label) can be monosaccharide-specific (e.g., specific for a terminal monosaccharide of a glycan) and/or specific to a particular disaccharide linkage type, and can provide for a means of identifying and quantifying glycopeptides that carry a particular monosaccharide or a particular monosaccharide (e.g., to provide a characteristic m/z peak offset to trigger MS2 acquisition of a particular glycopeptide analyte as it comes off an HPLC column during a LC/MS/MS run).

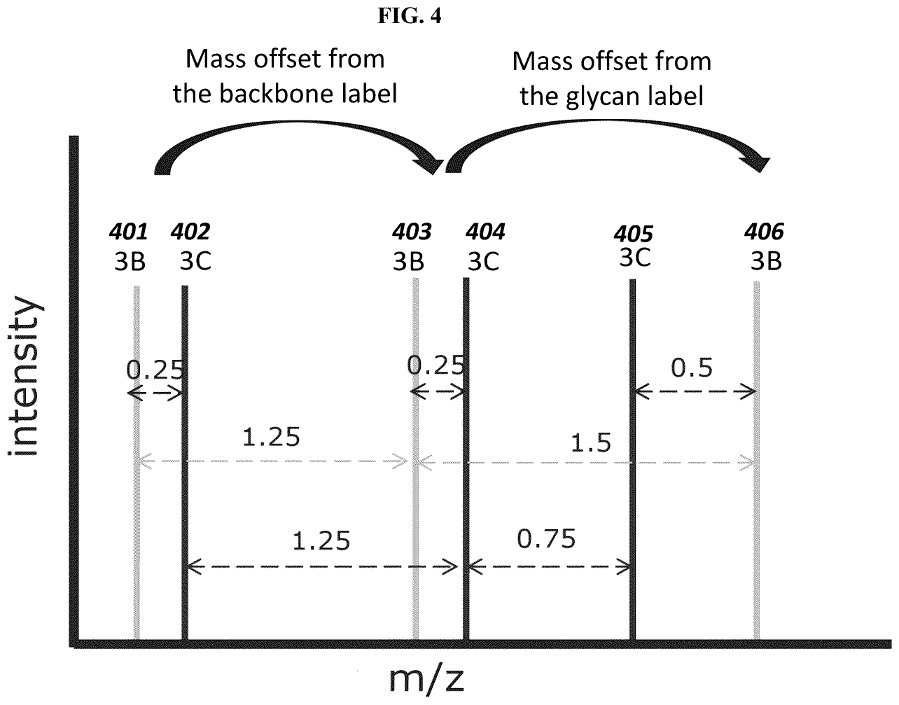

As shown in FIG. 4, while mass labeling (e.g., isotopic or isobaric labeling) on the peptide may create a mass offset from endogenous peptides (see e.g., the bars 403 and 404 corresponding to 3B and 3C in in the middle of the spectrum, which correspond to singly-labeled peptide) allowing species closely related in molecular weight to be distinguished, backbone labeling may still be insufficient for distinguishing glycan species similar in molecular weight. An illustrative example is shown in FIGS. 3A-3C, which illustrate examples of both monosaccharides and linked glycans that are difficult to resolve. FIG. 3A shows that a glycopeptide structure comprising one sialic acid (residual monoisotopic mass of 291.0954 Da) differs only by 1.0204 Da when compared to a glycopeptide structure comprising two fucose (residual monoisotopic mass of 292.1158 Da), indicating that a monosaccharide sialic acid modification is difficult to distinguish from a double fucose modification. FIG. 3B and FIG. 3C compare two common complex saccharide modifications (one di-sialylated biantennary complex type N-glycan and one biantennary complex type N-glycan with core-fucosylation and terminal sialyl Lewis A, respectively) which, while they comprise unique repertoires of monosaccharides, nonetheless are similar in monoisotopic mass (e.g., 2204.7724 vs 2205.79284), indicating that even complex saccharides of different composition can be difficult to distinguish.

Glycan-linked labels can be provided on top of glycopeptides labeled with a macromolecular backbone-linked isotopic tag as above to mitigate difficulties in distinguishing complex glycans by replacing monosaccharide constituents of complex glycans decorating glycopeptides with corresponding monosaccharides bearing a mass label (e.g., an isotopic label). For the purposes of illustration, the impact of example isotopic label modifications to the peptides of FIG. 3B and FIG. 3C are described in FIG. 4. Isotopically labeling two sialic acid residues (diamond-shaped residues representing two (3C13)Neu5Ac residues) in the molecule of FIG. 3B alongside a TMT6 label on the peptide backbone leads to a 2.75 Da offset from the endogenous glycopeptides at 4+ charge (compare 401 and 406 in FIG. 4; TMT0 vs TMT6 mass difference being predicted to be ˜5 Da for a peptide at 4+ charge leading to an m/z difference of 1.25; 2× (3C13)Neu5Ac vs unlabeled Neu5Ac mass difference predicted to be ˜6 Da for a peptide at 4+ charge leading to an m/z difference of 1.5). Labeling one sialic acid residue (diamond-shaped residue) alongside a TMT6 label on the peptide backbone in FIG. 3C leads to a 2 Da offset from the endogenous glycopeptides at 4+ charge (compare 402 and 405 in FIG. 4; TMT0 vs TMT6 mass difference being predicted to be ˜5 Da for a peptide at 4+ charge leading to an m/z difference of 1.25; 1× (3C13)Neu5Ac vs Neu5Ac mass difference predicted to be ˜3 Da for a peptide at 4+ charge leading to an m/z difference of 0.75). As shown in the graph depicted in FIG. 4, such glycan labeling (compare 405 and 406 in FIG. 4) can increase the mass difference between endogenous glycopeptides (compare 401 and 402 in FIG. 4) versus peptides with only a backbone label (see e.g., 403 and 404 in FIG. 4), further facilitating resolution of peptides of related mass/charge by separating them (405 and 406) from endogenous glycopeptides (401 and 406). Moreover, the glycan labeling is structure-specific that separates two glycopeptides of similar mass/charge further for better isolation (difference is 0.25 between 401 and 402 where it is 0.5 between 405 and 406).

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof. Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

The terms “subject,” “individual,” or “patient” are often used interchangeably herein. A “subject” can generally be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 15% of that number. The term “about” a range refers to that range minus 15% of its lowest value and plus 15% of its greatest value.

The terms “determining,” “measuring,” “evaluating,” “assessing,” “assaying,” and “analyzing” are often used interchangeably herein to refer to forms of measurement. The terms include determining if an element is present or not (for example, detection). These terms may include quantitative, qualitative, or quantitative and qualitative determinations. Assessing may be relative or absolute. “Detecting the presence of” may include determining the amount of something present in addition to determining whether it is present or absent depending on the context.

As used herein, the terms “treatment” or “treating” are generally used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying, or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The term tag or “isotopic tag” generally refers to a molecule of predetermined structure containing one or more stable heavy isotopes of an atom.

The term “heavy isotope” generally indicates an element with a heavier atomic weight than the highest abundance atomic weight counterpart. Useful heavy isotopes in mass spectrometry include ²H, ¹³C, ¹⁵N, ¹⁸O, and ³⁴S.

The term “isobaric labeling” or grammatical equivalents thereof generally refers to a technique by which macromolecules (e.g., peptides, lipids, or proteins) are labeled with chemical groups that have (at least nominally) identical mass (isobaric) but vary in terms of distribution of heavy isotopes in their structure. Such a technique finds usage in techniques such as mass spectrometry. Reagent molecules useful for isobaric labeling typically comprise an amine-, cysteine-, or carbonyl-reactive group and are designed such that the reagent molecule is cleaved at a specific linker region upon high-energy collision induced dissociation (CID) to yield reporter ions of varying masses.

As used herein, the term “mass spectrometry” (MS) generally refers to an analytical technique for the determination of the elemental composition, mass to charge ratio, absolute abundance or relative abundance of an analyte. Mass spectrometric techniques are useful for elucidating the composition or abundance of analytes, such as proteins, peptides and other chemical compounds. Mass spectrometry includes processes comprising ionizing analytes to generate charged species or species fragments, fragmentation of charged species or species fragments, such as product ions, and measurement of mass-to-charge ratios of charged species or species fragments, optionally including additional processes of isolation on the basis of mass to charge ratio, additional fragmentation processing, charge transfer processes, etc. Conducting a mass spectrometric analysis of an analyte (e.g., macromolecule or peptide) results in the generation of mass spectrometry data for example, comprising the mass-to-charge ratios and corresponding intensity data for the analyte and/or analyte fragments. Mass spectrometry data corresponding to analyte ion and analyte ion fragments is commonly provided as intensities of as a function of mass-to-charge (m/z) units representing the mass-to-charge ratios of the analyte ions and/or analyte ion fragments. Mass spectrometry commonly allows intensities corresponding to difference analytes to be resolved in terms of different mass to charge ratios. In tandem mass spectrometry (MS/MS or MS2), multiple sequences of mass spectrometry analysis are performed. For example, samples containing a mixture of proteins and peptides can be ionized and the resulting precursor ions separated according to their mass-to-charge ratio. Selected precursor ions can then be fragmented into product ions and further analyzed according to the mass-to-charge ratio of the fragments to infer connectivity among chemical groups in the species that comprise the precursor ions.

The term “LC MS” generally refers to an analytical chemistry technique that combines the physical separation capabilities of liquid chromatography (or HPLC) with the mass analysis capabilities of mass spectrometry (MS). Coupled chromatography-MS improve the performance of both chromatography and mass spectrometric systems alone. While liquid chromatography separates mixtures with multiple components, mass spectrometry provides spectral information that may help to identify (or confirm the suspected identity of) each separated component. MS is sensitive to low abundance analytes and provides selective detection, relieving the need for complete chromatographic separation. A typical LC MS system can involve a first stage of a liquid chromatography column (e.g., an adsorption chromatography column, a partition chromatography column, an ion-exchange chromatography column, a size-exclusion chromatography column, or an affinity chromatography column) coupled to a second stage mass spectrometer that ionizes analytes as they are resolved on the liquid chromatography column and records mass-to-charge ratio of the resultant species. An LC/MS/MS system will additionally allow for a second stage of mass spectrometric ionization and mass-to-charge ratio recording to record product ions formed from precursor ions that comprise particular isolation windows of the species resolved in the first stage mass spectrometer.

As used herein, the term “species” generally refers to a particular molecule, compound, ion, anion, atom, electron or proton. Species include isotopically labeled analytes, isotopic tagging reagents, isotopically labeled amino acids or isotopically labeled peptide, proteins, or lipids.

As used herein, the term “mass-to-charge ratio” or “mass/charge ratio” generally refers to the ratio of the mass of a species to the charge state of a species. The term “m/z unit” refers to a measure of the mass to charge ratio. The Thomson unit (abbreviated as Th) is an example of an m/z unit and is defined as the absolute value of the ratio of the mass of an ion (in Daltons) to the charge of the ion (with respect to the elemental charge).

As described herein, “isolation” or an “isolation window” generally refers to a range of ions, such as precursor ions that is selectively separated and fragmented, manipulated or isolated in a mass spectrometric trace.

The terms “peptide” and “polypeptide” generally are used synonymously in the present description, and generally refer to a class of compounds composed of amino acid residues chemically bonded together by amide bonds. Peptides can be polypeptides are polymeric compounds comprising at least two amino acid residues or modified amino acid residues. Modifications can be naturally occurring or non-naturally occurring, such as modifications generated by chemical synthesis. Modifications to amino acids in peptides include, but are not limited to, phosphorylation, glycosylation, lipidation, prenylation, sulfonation, hydroxylation, acetylation, methylation, methionine oxidation, alkylation, acylation, carbamylation, iodination, the addition of cofactors, and any combination thereof. Peptides can include proteins and further include compositions generated by degradation of proteins, for example by proteolytic digestion. Peptides and polypeptides can be generated by substantially complete digestion or by partial digestion of proteins. Polypeptides include, for example, polypeptides comprising 2 to 100 amino acid units, optionally for some embodiments 2 to 50 amino acid units and, optionally for some embodiments 2 to 20 amino acid units and, optionally for some embodiments 2 to 10 amino acid units.

An “amino acid” generally refers to an organic compound containing an amino group (NH2), a carboxylic acid group (COOH), and any of various side chain groups. Amino acids may be characterized by the basic formula NH2CHRCOOH wherein R is the side chain group. Natural amino acids are those amino acids which are produced in nature, such as isoleucine, alanine, leucine, asparagine, lysine, aspartic acid, methionine, cysteine, phenylalanine, glutamic acid, threonine, glutamine, tryptophan, glycine, valine, proline, serine, tyrosine, arginine, and histidine as well as ornithine and selenocysteine.

As used herein, the term “precursor ion” is generally used herein to refer to an ion which is produced during an ionization stage of mass spectrometry analysis, including the MS1 ionization stage of MS/MS analysis.

As used herein, the terms “product ion” and “secondary ion” are generally used interchangeably in the present description and generally refer to an ion which is produced during an ionization or fragmentation process during mass spectrometry analysis (e.g., during an MS2 ionization stage of an MS/MS analysis).

As used herein, the terms “tag” and “label” are generally used interchangeably.

The section headings used herein are for organizational purposes only and are not to be construed as limiting the subject matter described.

### Biological Samples

Biological samples may be used as a source of proteins for quantitation or identification. In some aspects, biological samples may be used as a source of lipids for quantitation or identification. The sample may be a biological sample. Multiple biological samples may be used. Two or more biological samples may be used. Three or more biological samples may be used. Four or more biological samples may be used. Five or more biological samples may be used. Six or more biological samples may be used. Seven or more biological samples may be used. Eight or more biological samples may be used. Nine or more biological samples may be used. Ten or more biological samples may be used. The biological samples may be collected at the same time, or at different times. The biological samples may be collected at the same hour or same day. The biological samples may be collected at different hours or different days. The biological sample may be cell-free or substantially cell-free. The biological sample may undergo a sample preparation method. The biological sample may be digested. The biological sample may be protease digested. The biological sample may be collected or stored in a container. The biological sample may not be collected or stored in a container.

The biological sample may be blood. The biological sample may be serum. The biological sample may be plasma. The biological sample may include blood, serum, or plasma, or a combination thereof. The biological sample may be urine. The biological sample may be tears. The biological sample may be semen. The biological sample may be milk. The biological sample may be vaginal fluid. The biological sample may be mucous. The biological sample may be saliva. The biological sample may be sweat. The biological sample may include urine, tears, semen, milk, vaginal fluid, mucus, saliva, or sweat, or combinations thereof. The biological sample may include blood, serum, plasma, urine, tears, semen, milk, vaginal fluid, mucus, saliva, or sweat, or combinations thereof.

The biological sample may be a biofluid. Examples of a biofluid include blood, serum, or plasma. The biofluid may be blood, serum, plasma, urine, tears, semen, milk, vaginal fluid, mucus, saliva, or sweat, or combinations thereof.

The biological sample may include an endogenous protein or a proteome or individual constituents thereof. The endogenous protein or proteome may be produced inside an organism or cell. The endogenous protein may be produced inside a subject. The biological sample may include multiple endogenous proteins. The biological sample may include one or more endogenous proteins. The biological sample may include two or more endogenous proteins. The biological sample may include three or more endogenous proteins. The biological sample may include four or more endogenous proteins. The biological sample may include five or more endogenous proteins. The biological sample may include six or more endogenous proteins. The biological sample may include seven or more endogenous proteins. The biological sample may include eight or more endogenous proteins. The biological sample may include nine or more endogenous proteins. The biological sample may include ten or more endogenous proteins. For example, the biological sample may comprise endogenous protein A, endogenous protein B, and endogenous protein C. As another example, the biological sample may comprise endogenous protein A, endogenous protein B, endogenous protein C, endogenous protein D, and endogenous protein E.

The endogenous protein may comprise a post-translational modification (PTM). The PTM may comprise glycosylation. The glycosylation may N-linked glycosylation. The glycosylation may comprise asparagine-linked glycosylation. The PTM may comprise a glycan. The PTM may comprise ubiquitination. The PTM may comprise a ubiquitin. The PTM may comprise phosphorylation. The PTM may comprise a phosphate. The PTM may comprise acetylation. The PTM may be comprise an acetyl. The PTM may comprise glycosylation, ubiquitination, phosphorylation, or acetylation, or a combination thereof. The PTM may comprise a glycan, ubiquitin, phosphate, or acetyl, or a combination thereof. The endogenous protein may comprise a glycoprotein.

The biological sample may include an endogenous lipid. The biological sample may include one or more endogenous lipids. The biological sample may include two or more endogenous lipids. The biological sample may include three or more endogenous lipids. The biological sample may include four or more endogenous lipids. The biological sample may include five or more endogenous lipids. The biological sample may include six or more endogenous lipids. The biological sample may include seven or more endogenous lipids. The biological sample may include eight or more endogenous lipids. The biological sample may include nine or more endogenous lipids. The biological sample may include ten or more endogenous lipids. For example, the biological sample may comprise endogenous lipid A, endogenous lipid B, and endogenous lipid C. As another example, the biological sample may comprise endogenous lipid A, endogenous lipid B, endogenous lipid C, endogenous lipid D, and endogenous lipid E.

The biological sample may include an endogenous biomolecule. The biological sample may include one or more endogenous biomolecules. The biological sample may include two or more endogenous biomolecules. The biological sample may include three or more endogenous biomolecules. The biological sample may include four or more endogenous biomolecules. The biological sample may include five or more endogenous biomolecules. The biological sample may include six or more endogenous biomolecules. The biological sample may include seven or more endogenous biomolecules. The biological sample may include eight or more endogenous biomolecules. The biological sample may include nine or more endogenous biomolecules. The biological sample may include ten or more endogenous biomolecules. For example, the biological sample may comprise endogenous biomolecule A, endogenous biomolecule B, and endogenous biomolecule C. As another example, the biological sample may comprise endogenous biomolecule A, endogenous biomolecule B, endogenous biomolecule C, endogenous biomolecule D, and endogenous biomolecule E.

The biological sample may be taken from a subject. The subject may be a human. The subject may be male or female. The subject may be a vertebrate. The subject may be a mammal. The subject may have a disease state. The subject may have a non-disease state. The subject may be in a healthy state.

The subject may be obtained for purposes of identifying a disease state in the subject. The subject may be obtained for purposes unrelated to identifying a disease state in the subject. The subject may be suspected as having the disease state or as not having the disease state in the subject. The methods described here may be used to confirm or refute the suspected disease state in the subject.

### Processing or Enriching of a Biological Sample

The biological sample may be enriched, processed, or purified in any order. The biological sample may be enriched or purified for peptides. The biological sample may be enriched or purified for peptides comprising a PTM. The PTM may comprise a glycan, ubiquitin, phosphate, or acetyl, or a combination thereof.

The biological sample may be processed by enzymatic treatment (e.g., to generate macromolecular fragments more amenable to analysis, to remove particular glycans, or to add particular glycans). The enzymatic treatment can include a protease (e.g., an endoprotease). The protease can be an endoprotease or exoprotease. An exoprotease can comprise Carboxypeptidase A, Carboxypeptidase B (which is specific for lysine or arginine), Carboxypeptidase P, Carboxypeptidase Y, Cathepsin C (which removed an N-terminal dipeptide except when N-terminal amino acid is lysine or arginine, or when 2^{nd} or 3^{rd} amino acid from N-terminal is proline), or Chymotrypsin (which is specific for phenylalanine, tryptophan, and tyrosine). The protease can include a protease with a define amino acid residue specificity (e.g., cut after a specific residue or sequence of residues). An endoproteinase can comprise Clostripain (which is specific for arginine), Elastase (which is specific for Alanine, Valine, Serine, Glycine, Leucine, or Isoleucine), Arg-C (which is specific for Arginine), Glu-C (which is specific for glutamic acid), Lys-C (which is specific for lysine), glutamyl endopeptidase (which is specific for glutamic acid), Kallikrein (which is specific for lysine or arginine), Papain (which is specific for Lysine or Arginine followed by a hydrophobic residue), Pepsin (which is specific for Leucine, Phenylalanine, Tryptophan or Tyrosine), Proteinase K (which is specific for aliphatic and aromatic amino acids), Subtilisin (which is specific for hydrophobic amino acids), or Trypsin (which is specific for lysine or arginine). Treatment with any of the aforementioned proteases can generate peptide fragments having predetermined C-terminal residues based on their specificities. The enzymatic treatment can include a glycosidase. The glycosidase can include any of the enzyme activities or enzymes described in Table 1, or a combination thereof. The enzymatic treatment can include a glycosyltransferase. The glycosyltransferase can include any of the enzyme activities or enzymes described in Table 2, or a combination thereof.

The biological sample may be enriched or purified using a suitable chromatographic method. The biological sample may be enriched or purified using hydrophilic interaction liquid chromatography (HILIC). The biological sample may be enriched or purified using liquid chromatography. The biological sample may be enriched or purified using solid-phase chromatography. The biological sample may be enriched or purified using column chromatography, including affinity chromatography, ion exchange chromatography, and size exclusion chromatography. The biological sample may be enriched or purified using chromatography, HILIC, liquid chromatography, solid-phase chromatography, column chromatography, including affinity chromatography, ion exchange chromatography, and size exclusion chromatography, or a combination thereof.

The biological sample may be enriched, processed, or purified for more than 10 minutes, more than 30 minutes, more than 1 hour, more than 1.5 hours, more than 2 hours, more than 2.5 hours, more than 3 hours, more than 3.5 hours, more than 4 hours, more than 4.5 hours, more than 5 hours, more than 6 hours, more than 7 hours, more than 8 hours, more than 9 hours, or more than 10 hours. The biological sample may be enriched or purified for less than 10 minutes, less than 30 minutes, less than 1 hour, less than 1.5 hours, less than 2 hours, less than 2.5 hours, less than 3 hours, less than 3.5 hours, less than 4 hours, less than 4.5 hours, less than 5 hours, less than 6 hours, less than 7 hours, less than 8 hours, less than 9 hours, or less than 10 hours. The biological sample may be enriched or purified for a fixed time. The biological sample may be enriched or purified for a variable time.

The biological sample may be enriched, processed, or purified prior to generating the double-labeled peptide standard. The biological sample may be enriched or purified while generating the double-labeled peptide standard. The biological sample may not be enriched or purified prior generating the double-labeled peptide standard. The biological sample may be enriched or purified for more than 10 minutes prior to generating the double-labeled peptide standard, more than 30 minutes, more than 1 hour, more than 1.5 hours, more than 2 hours, more than 2.5 hours, more than 3 hours, more than 3.5 hours, more than 4 hours, more than 4.5 hours, more than 5 hours, more than 6 hours, more than 7 hours, more than 8 hours, more than 9 hours, or more than 10 hours prior to generating the double-labeled peptide standard. The biological sample may be enriched or purified for less than 10 minutes prior to generating the double-labeled peptide standard, less than 30 minutes, less than 1 hour, less than 1.5 hours, less than 2 hours, less than 2.5 hours, less than 3 hours, less than 3.5 hours, less than 4 hours, less than 4.5 hours, less than 5 hours, less than 6 hours, less than 7 hours, less than 8 hours, less than 9 hours, or less than 10 hours prior to generating the double-labeled peptide standard.

The biological sample may be enriched or purified for lipids. The biological sample may be enriched or purified prior to generating the double-labeled lipid standard. The biological sample may be enriched or purified while generating the double-labeled lipid standard. The biological sample may not be enriched or purified prior to generating the double-labeled lipid standard. The biological sample may be enriched or purified for more than 10 minutes prior to generating the double-labeled lipid standard, more than 30 minutes, more than 1 hour, more than 1.5 hours, more than 2 hours, more than 2.5 hours, more than 3 hours, more than 3.5 hours, more than 4 hours, more than 4.5 hours, more than 5 hours, more than 6 hours, more than 7 hours, more than 8 hours, more than 9 hours, or more than 10 hours prior to generating the double-labeled lipid standard. The biological sample may be enriched or purified for less than 10 minutes prior to generating the double-labeled lipid standard, less than 30 minutes, less than 1 hour, less than 1.5 hours, less than 2 hours, less than 2.5 hours, less than 3 hours, less than 3.5 hours, less than 4 hours, less than 4.5 hours, less than 5 hours, less than 6 hours, less than 7 hours, less than 8 hours, less than 9 hours, or less than 10 hours prior to generating the double-labeled lipid standard.

The biological sample may be enriched or purified for biomolecules. The biological sample may be enriched or purified prior to generating the double-labeled biomolecule standard. The biological sample may be enriched or purified while generating the double-labeled biomolecule standard. The biological sample may not be enriched or purified prior to generating the double-labeled biomolecule standard. The biological sample may be enriched or purified for more than 10 minutes prior to generating the double-labeled biomolecule standard, more than 30 minutes, more than 1 hour, more than 1.5 hours, more than 2 hours, more than 2.5 hours, more than 3 hours, more than 3.5 hours, more than 4 hours, more than 4.5 hours, more than 5 hours, more than 6 hours, more than 7 hours, more than 8 hours, more than 9 hours, or more than 10 hours prior to generating the double-labeled biomolecule standard. The biological sample may be enriched or purified for less than 10 minutes prior to generating the double-labeled biomolecule standard, less than 30 minutes, less than 1 hour, less than 1.5 hours, less than 2 hours, less than 2.5 hours, less than 3 hours, less than 3.5 hours, less than 4 hours, less than 4.5 hours, less than 5 hours, less than 6 hours, less than 7 hours, less than 8 hours, less than 9 hours, or less than 10 hours prior to generating the double-labeled biomolecule standard.

A portion of the biological sample may be enriched or purified for generating the double-labeled peptide standard, the double-labeled lipid standard, or the double-labeled biomolecule standard. For example, FIG. 1 shows a 90% portion of the biological sample being enriched or purified and a 10% portion of the biological sample not being enriched or purified. In some aspects, a majority of the biological sample may be enriched or purified. In some aspects, a minority of the biological sample may be enriched or purified. In some aspects, at least 5%, 10%, 20%, 30%, 40%, 50%, 60%, 70%, 80%, 90%, 95%, or at least 99% of the biological sample may be enriched or purified. In some aspects, at most 99%, 95%, 90%, 80%, 70%, 60%, 50%, 40%, 30%, 20%, 10%, or at most 5% of the biological sample may be enriched or purified. A majority or a minority of the biological sample may be enriched or purified. The enriched or purified portion of the biological sample may be half of the biological sample, less than half, or more than half of the biological sample, or combinations thereof.

### Glycopeptide Enrichment

Glycosylation is a posttranslational modification that may affect many biological functions. Glycoproteomics may be used to study of protein glycosylation. Due to low abundances of glycopeptides (peptides that are glycosylated) in biological samples, it is useful to enrich glycopeptides from non-glycopeptides for in-depth analyses. It may be useful to remove salts from the fluid in which the glycopeptides are present in order to facilitate analysis in further testing. This may be due to limitations of analytical equipment to process samples that contain excessive ions, concerns about the stability of the glycoprotein in a high salt environment, or other issues. The same may be true of the buffer and solvent in which the glycopeptide is contained. Therefore, it is useful to change or remove components of the buffer or change the solvent in which the glycoprotein is present as well.

Hydrophilic interaction liquid chromatography (HILIC) is a chromatographic technique that is useful to enrich glycopeptides for glycoproteomic studies. In some workflows, digests of peptides are desalted by reverse phase separation. HILIC may then employed to enrich glycopeptides from the desalted peptide mixture. Such two-step procedures can be time-consuming and may cause sample loss during each step. Low abundances of the glycopeptides make the loss of sample from multiple steps enrichments a significant issue. In addition, reducing assay steps may, in some cases, be useful for streamlining processes to collect large data sets in glycoproteomic studies.

Aspects of the present disclosure provide for a method or device for the enrichment of glycosylated peptides and proteins. In some aspects of this invention is a device in which a HILIC material is placed before a reverse phase chromatography material. In some aspects of this invention is a device in which a HILIC material is placed after a reverse phase chromatography material. Based on the physical properties between peptide and glycopeptide, this may allow for the simultaneous desalting and enrichment of glycopeptides. This may, in some cases, be achieved in one device. By having the materials in a single device, fewer transfers take place and less product (e.g., glycopeptides) is lost along the way of purification.

Aspects of the present disclosure provide for a method or device for the enrichment of glycosylated peptides and proteins. In some aspects of this invention a device in which a HILIC material is sandwiched by a reverse phase material is disclosed. Based on the physical properties between peptide and glycopeptide, this may allow for the simultaneous desalting and enrichment of glycopeptides. This may, in some cases, be achieved in one device.

Similar ideas have described using heterogenous HILIC materials. More specifically, materials that has both hydrophobic and hydrophilic properties in different region of the material. However, in aspects of this disclosure the chromatography materials are differentiated by their homogenous composition. In some aspects of the present disclosure the ratio, amounts, and properties of reverse phase materials and HILIC materials may be adjusted and optimized for specific applications.

### Advantages of Workflows and Methods of the Disclosure

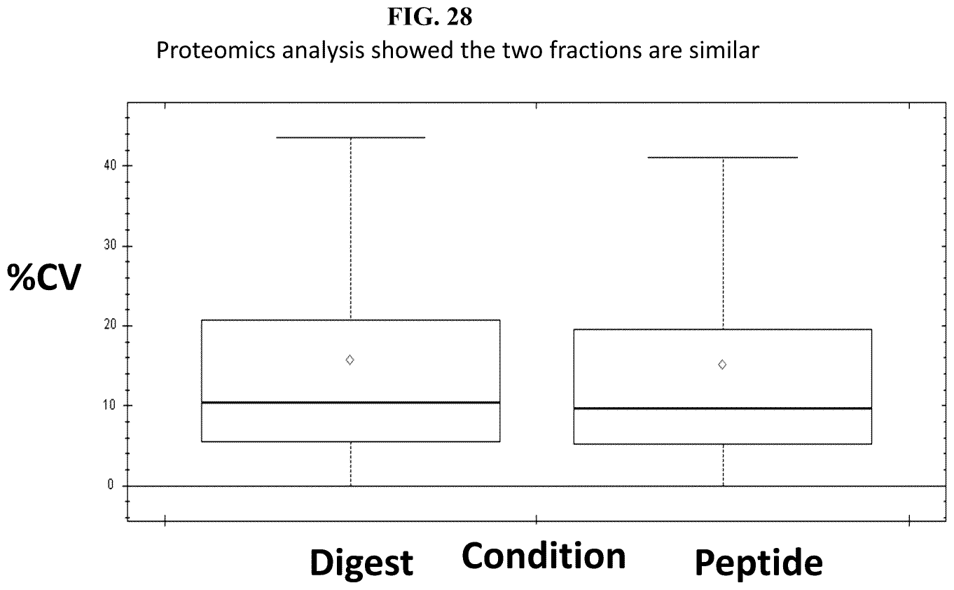

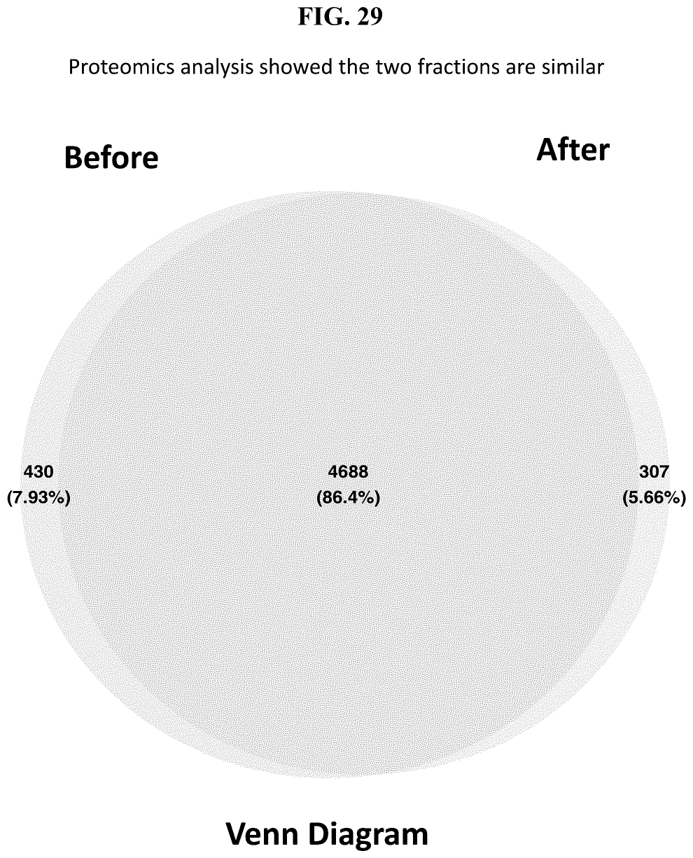

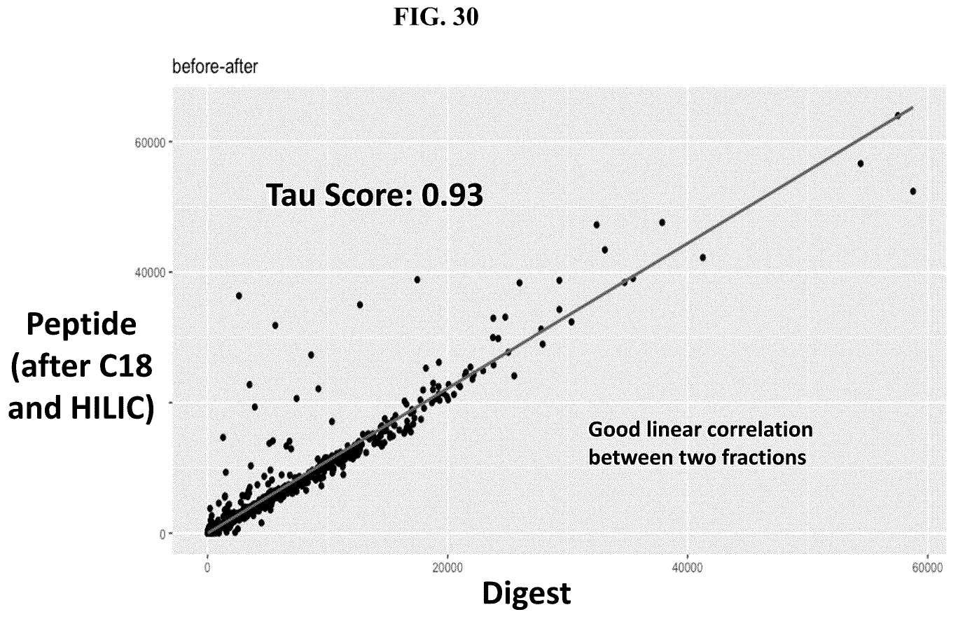

As shown in FIG. 28, the narrow coefficient variation value generated from workflow 2 illustrates high reproducibility between the digest fraction and peptide fraction(s). The Tau score of 0.93 and the strong linear correlation (FIG. 30) between the digest fraction and peptide fraction also shows evidence for high reproducibility. The high reproducibility demonstrates the high reliability of workflow 2. Proteomics analysis shows the detection of an overlap of 4688 unique precursor identities between the digest and workflow 1. In addition to the overlapping precursor identities, the method of workflow 2 resulted in the detection of 430 more precursors and the method of the digest only resulted in the detection of 307 more precursors (FIG. 29). In some embodiments, the digest and the peptide fraction from workflow 2 have at least about 60%, at least about 65%, at least about 70%, at least about 75%, at least about 80%, at least about 85%, at least about 90%, at least about 95%, or more overlapping precursor identities. In some embodiments, a peptide fraction of biofluid and a peptide fraction of the same biofluid post-separation have at least about 60%, at least about 65%, at least about 70%, at least about 75%, at least about 80%, at least about 85%, at least about 90%, at least about 95%, or more overlapping precursor identities. In some embodiments, a peptide fraction of biofluid and a peptide fraction of the same biofluid that has been flowed through stationary phases have at least about 60%, at least about 65%, at least about 70%, at least about 75%, at least about 80%, at least about 85%, at least about 90%, at least about 95%, or more overlapping precursor identities. In some embodiments, a peptide fraction of biofluid and a peptide fraction of the same biofluid that has been flowed through one, two, three, or more stationary phases have at least about 60%, at least about 65%, at least about 70%, at least about 75%, at least about 80%, at least about 85%, at least about 90%, at least about 95%, or more overlapping precursor identities.

As shown in FIG. 29, the peptide has an 86.4% match between the digest and the peptide fraction from workflow 2. In some embodiments, the digest and the peptide fraction from workflow 2 have at most about 95%, at most about 90%, at most about 85%, at most about 80%, at most about 75%, at most about 70%, at most about 65%, at most about 60%, or less overlapping precursor identities. In some embodiments, a peptide fraction of biofluid and a peptide fraction of the same biofluid post-separation have at most about 95%, at most about 90%, at most about 85%, at most about 80%, at most about 75%, at most about 70%, at most about 65%, at most about 60%, or less overlapping precursor identities. In some embodiments, a peptide fraction of biofluid and a peptide fraction of the same biofluid that has been flowed through stationary phases have at most about 95%, at most about 90%, at most about 85%, at most about 80%, at most about 75%, at most about 70%, at most about 65%, at most about 60%, or less or more overlapping precursor identities. In some embodiments, a peptide fraction of biofluid and a peptide fraction of the same biofluid that has been flowed through one, two, three, or more stationary phases have at most about 95%, at most about 90%, at most about 85%, at most about 80%, at most about 75%, at most about 70%, at most about 65%, at most about 60%, or less overlapping precursor identities.

In some embodiments, a peptide fraction of biofluid and a peptide fraction of the same biofluid post-separation have at most about 5%, at most about 7%, at most about 10%, at most about 12%, at most about 15%, at most about 20%, or more non-overlapping precursor identities. In some embodiments, a peptide fraction of biofluid and a peptide fraction of the same biofluid post-separation have at least about 10%, at least about 7.5%, at least about 5%, at least about 2.5%, at least about 1%, or less non-overlapping precursor identities.

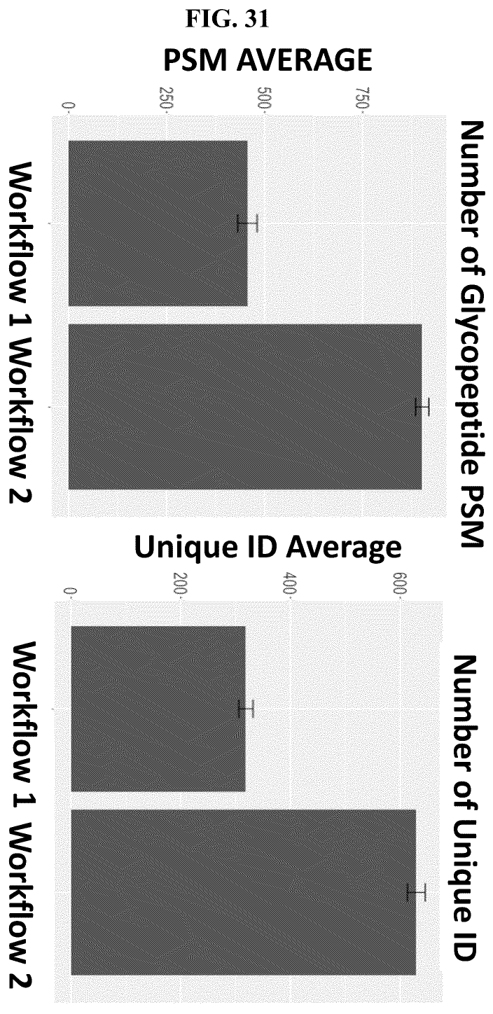

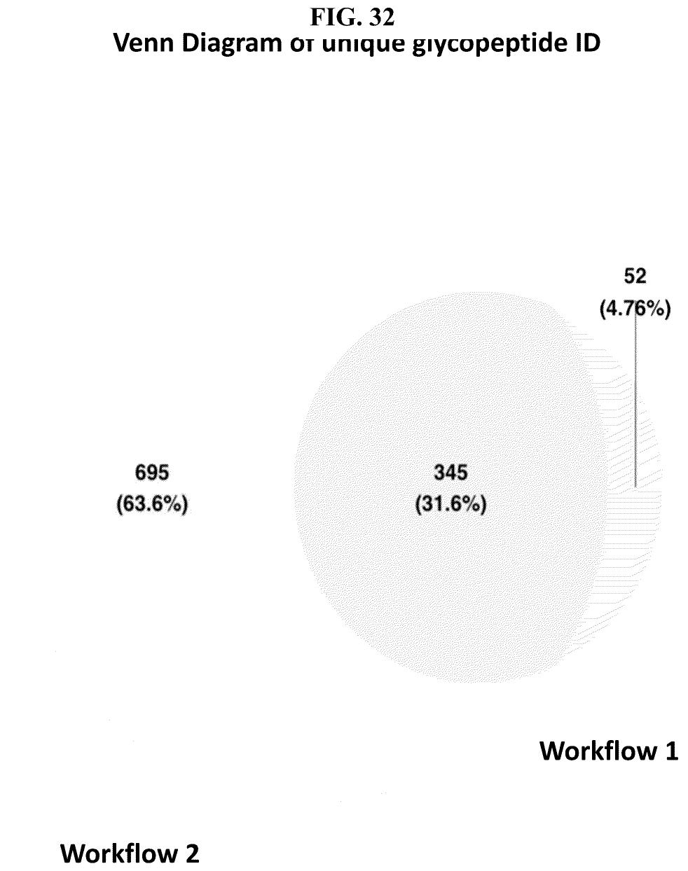

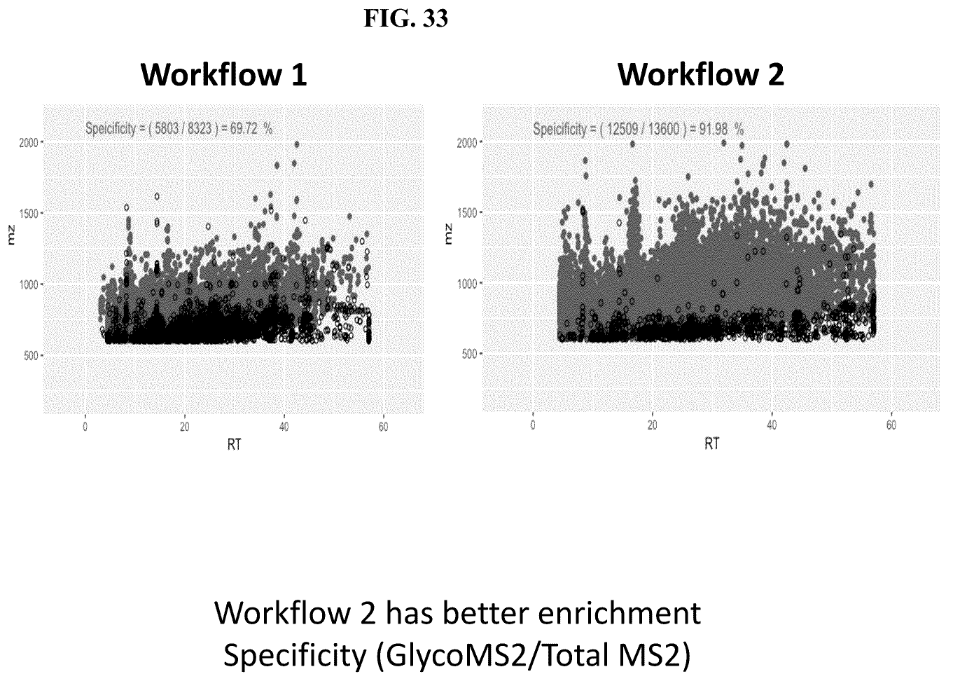

As shown in FIG. 31, the number of glycopeptide peptide spectrum match (PSM) and the number of unique glycopeptide identifications of workflow 1 and workflow 2 are compared. Workflow 2 resulted in an average of approximately 875 glycopeptide PSMs while workflow 1 resulted in an average of approximately 450 glycopeptide PSMs, showing that workflow 2 was able to identify double the number of glycopeptide PSMs compared to workflow 1. FIG. 32 illustrates a unique glycopeptide identification overlap of 345 species (31.6%) between workflow 1 and workflow 2. In addition to the overlapping unique glycopeptide identifications, workflow 2 resulted in the detection of 695 (63.6%) more unique glycopeptide identifications while workflow 1 resulted in the detection of 52 (4.76%) more unique glycopeptide identifications. Further analysis of MS2 spectra of FIG. 33 demonstrates the high specificity of workflow 2 (e.g., 12509 species of 13600 species are glycopeptides) compared to the specificity of workflow 1 (e.g., 5803 species of 8323 species are glycopeptides). In some embodiments, the glycopeptide enrichment specificity of workflow 1, workflow 2, or one device is at least about 60%, at least about 65%, at least about 70%, at least about 75%, at least about 80%, at least about 85%, at least about 90%, at least about 95%, at least about 97%, or more. In some embodiments, the glycopeptide enrichment specificity of workflow 1, workflow 2, or one device is at most about 99%, at most about 95%, at most about 90%, at most about 85%, at most about 80%, at most about 75%, at most about 70%, at most about 65%, or less. In some embodiments, the glycopeptide enrichment specificity is higher for the same biofluid following one device than for the same biofluid following workflow 1. In some embodiments, the glycopeptide enrichment specificity is higher for the same biofluid following one device than for the same biofluid following workflow 2. In some embodiments, the glycopeptide enrichment specificity is higher for the same biofluid following workflow 2 than for the same biofluid following workflow 1.

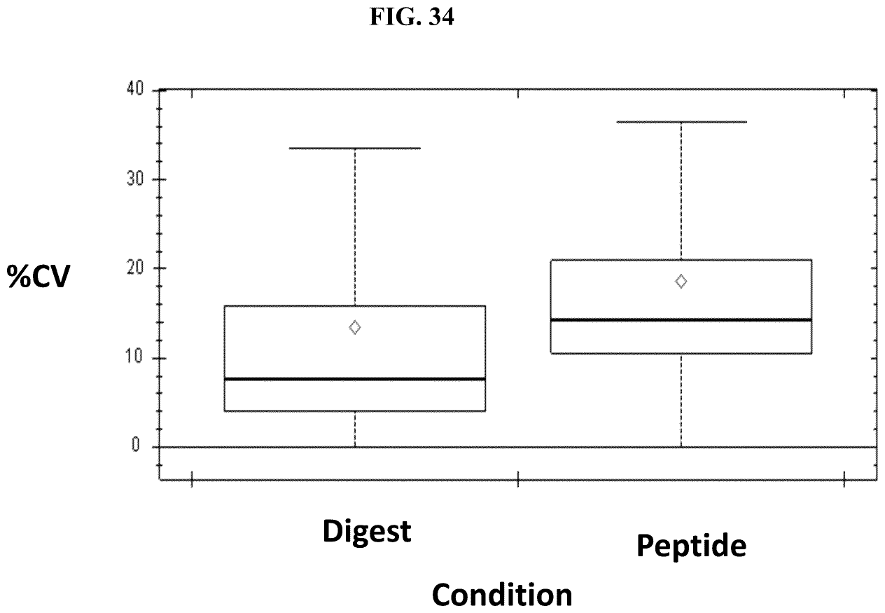

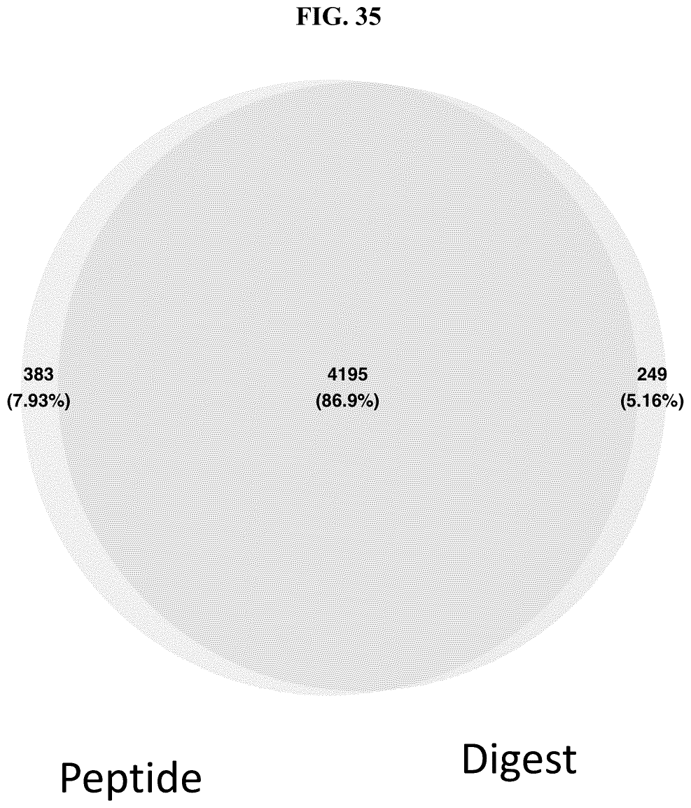

As shown in FIG. 34, the narrow coefficient variation value generated from one device illustrates high reproducibility between the digest fraction and peptide fraction(s). The Tau score of 0.92 and the strong linear correlation between the digest fraction and the peptide fraction also shows evidence for high reproducibility. Further, these results demonstrate that the one device method does not negatively affect the quality or quantity of detectable and identifiable peptides. The high reproducibility demonstrates the high reliability of the one device method. Proteomics analysis shows the detection of an overlap of 4195 unique precursor identities between the digest and one device peptide fraction. In addition to the overlapping precursor identities, the one device method resulted in the detection of 383 more precursors and the method of the digest only resulted in the detection of 249 more precursors (FIG. 35).

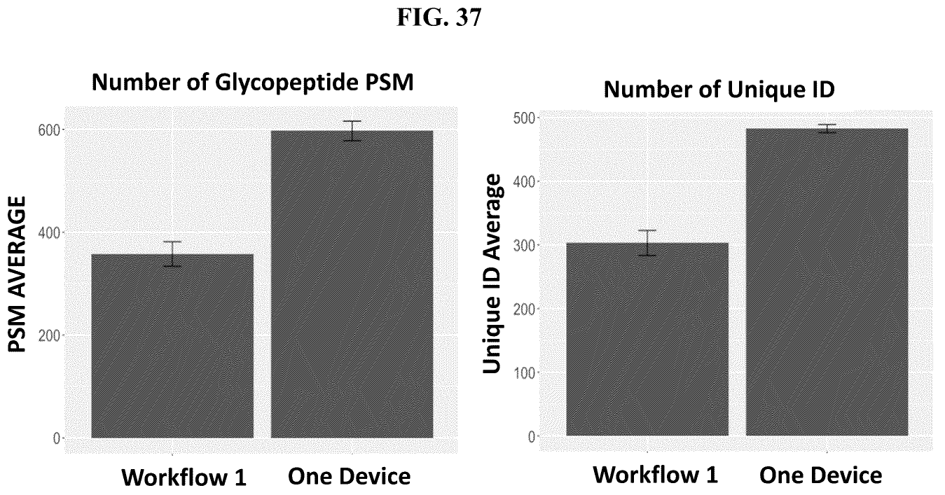

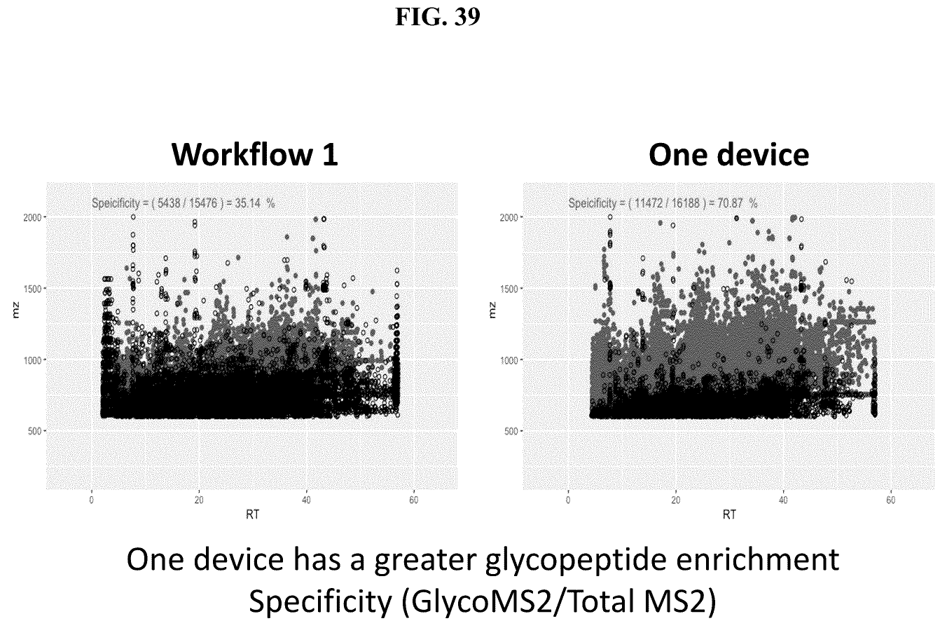

The number of glycopeptide peptide spectrum match (PSM) and the number of unique glycopeptide identifications of the one device method and workflow 1 are compared in FIG. 37. The one device method resulted in an average of approximately 600 glycopeptide PSMs while workflow 1 resulted in an average of approximately 360 glycopeptide PSMs, showing that the one device method was able to identify nearly double the number of glycopeptides PSMs compared to workflow 1. The number of overlapping unique glycopeptide IDs between the one device method and workflow 1 is 375. The one device method resulted in the detection of 300 additional unique glycopeptide IDs whereas workflow 1 resulted in the detection of 59 additional unique glycopeptide IDs. These results demonstrate that the one device had a near 6-fold improvement compared to workflow 1. Further analysis of MS2 spectra of FIG. 39 demonstrates the high specificity of one device (e.g., 11472 species of 16188 species are glycopeptides) compared to the specificity of workflow 1 (e.g., 5438 species of 15476 species are glycopeptides). In some embodiments, the digest and the peptide fraction from one device have at least about 60%, at least about 65%, at least about 70%, at least about 75%, at least about 80%, at least about 85%, at least about 90%, at least about 95%, or more overlapping precursor identities. In some embodiments, a peptide fraction of biofluid and a peptide fraction of the same biofluid post-separation have at least about 60%, at least about 65%, at least about 70%, at least about 75%, at least about 80%, at least about 85%, at least about 90%, at least about 95%, or more overlapping precursor identities. In some embodiments, a peptide fraction of biofluid and a peptide fraction of the same biofluid that has been flowed through stationary phases have at least about 60%, at least about 65%, at least about 70%, at least about 75%, at least about 80%, at least about 85%, at least about 90%, at least about 95%, or more overlapping precursor identities. In some embodiments, a peptide fraction of biofluid and a peptide fraction of the same biofluid that has been flowed through one, two, three, or more stationary phases have at least about 60%, at least about 65%, at least about 70%, at least about 75%, at least about 80%, at least about 85%, at least about 90%, at least about 95%, or more overlapping precursor identities.

As shown in FIG. 35, the peptide has an 86.9% match between the digest and the peptide fraction from one device. In some embodiments, the digest and the peptide fraction from one device have at most about 95%, at most about 90%, at most about 85%, at most about 80%, at most about 75%, at most about 70%, at most about 65%, at most about 60%, or less overlapping precursor identities. In some embodiments, a peptide fraction of biofluid and a peptide fraction of the same biofluid post-separation have at most about 95%, at most about 90%, at most about 85%, at most about 80%, at most about 75%, at most about 70%, at most about 65%, at most about 60%, or less overlapping precursor identities. In some embodiments, a peptide fraction of biofluid and a peptide fraction of the same biofluid that has been flowed through stationary phases have at most about 95%, at most about 90%, at most about 85%, at most about 80%, at most about 75%, at most about 70%, at most about 65%, at most about 60%, or less or more overlapping precursor identities. In some embodiments, a peptide fraction of biofluid and a peptide fraction of the same biofluid that has been flowed through one, two, three, or more stationary phases have at most about 95%, at most about 90%, at most about 85%, at most about 80%, at most about 75%, at most about 70%, at most about 65%, at most about 60%, or less overlapping precursor identities.

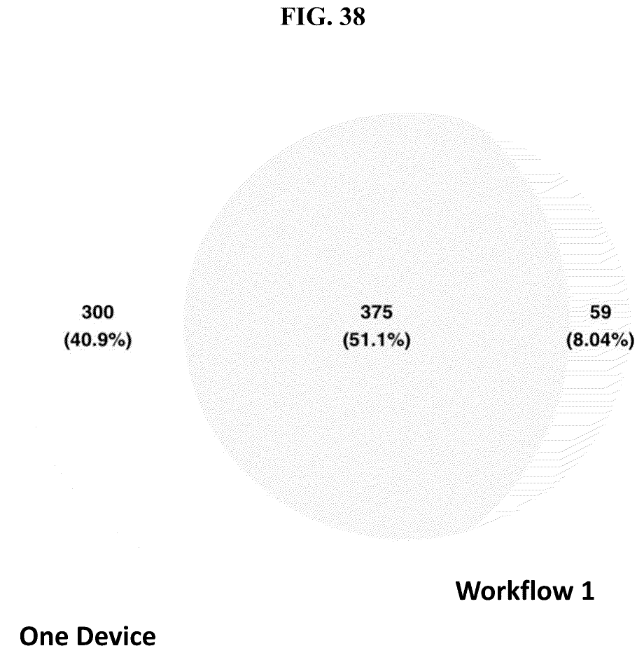

As shown in FIG. 38, there is a 51.1% overlap of unique glycopeptide identifications of workflow 1 and one device. In some embodiments, a peptide fraction of biofluid following workflow 1 and a peptide fraction of the same biofluid following one device have at most about 5%, at most about 7%, at most about 10%, at most about 12%, at most about 15%, at most about 20%, or more non-overlapping precursor identities. In some embodiments, a peptide fraction of biofluid following workflow 1 and a peptide fraction of the same biofluid following one device have at least about 10%, at least about 7.5%, at least about 5%, at least about 2.5%, at least about 1%, or less non-overlapping precursor identities.

### Advantages Over Existing Products

In some embodiments, the workflows and devices of the present disclosure have certain advantages over glycopeptide enrichment kits and devices that are currently commercially available. In some embodiments, the advantages include higher glycopeptide specificity. In some embodiments, the advantages include less sample loss due to removing a drying and reconstitution step for preparing a biofluid. In some embodiments, the advantages include removing a need for a drying and reconstitution step for preparing a biofluid. In some embodiments, the advantages include harvesting the peptide fraction for general proteomics purpose. In some embodiments, the advantages include improved separation of peptides, glycopeptides, and phosphopeptides. In some embodiments, the advantages include improved separation of peptides and glycopeptides. In some embodiments, the advantages include a quicker method. In some embodiments, the advantages include a quicker method due to removing a drying and reconstitution step.

### Device for Enrichment

### Device Body

Described herein is a device that may, in some cases, be used for the enrichment of glycopeptides. The device can have any number of stationary phases. The device may have one, two, three, four, five, six, or more stationary phases. The device may have any number of mobile phases. Any number of mobile phases may be flowed through the device. The device may have one, two, three, four, five, six, or more mobile phases flowed through. The stationary phases and the mobile phases may be as described elsewhere herein.

The device comprises a device body that defines an internal volume. The device body may be or include a pipette, pipette tip, or chromatography column. The device body may comprise plastic, composite, or metal materials. It may vary in size. The volume defined by the body of the device may, in some cases, be no more than about 0.1 μL, no more than about 0.5 μL, no more than about 1 μL, no more than about 10 μL, no more than about 20 μL, no more than about 50 μL, no more than about 75 μL, no more than about 100 μL, no more than about 200 μL, no more than about 300 μL, no more than about 400 μL, no more than about 500 μL, no more than about 750 μL, no more than about 1 mL, no more than about 2 mL, no more than about 3 mL, no more than about 4 mL, no more than about 5 mL, no more than about 6 mL, no more than about 7 mL, no more than about 8 mL, no more than about 9 mL, no more than about 10 mL, no more than about 15 mL, no more than about 20 mL, no more than about 25 mL, no more than about 30 mL, no more than about 35 mL, no more than about 40 mL, no more than about 45 mL, no more than about 50 mL, no more than about 60 mL, no more than about 70 mL, no more than about 80 mL, no more than about 90 mL, no more than about 100 mL, no more than about 150 mL, no more than about 200 mL, no more than about 250 mL, no more than about 300 mL, no more than about 400 mL, no more than about 500 mL, no more than about 750 mL, no more than about 1 L, or no more than about 1.5 L. The volume defined by the body of the device may, in some cases, be greater than about 0.1 μL, greater than about 0.5 μL, greater than about 1 μL, greater than about 10 μL, greater than about 20 μL, greater than about 50 μL, greater than about 75 μL, greater than about 100 μL, greater than about 200 μL, greater than about 300 μL, greater than about 400 μL, greater than about 500 μL, greater than about 750 μL, greater than about 1 mL, greater than about 2 mL, greater than about 3 mL, greater than about 4 mL, greater than about 5 mL, greater than about 6 mL, greater than about 7 mL, greater than about 8 mL, greater than about 9 mL, greater than about 10 mL, greater than about 15 mL, greater than about 20 mL, greater than about 25 mL, greater than about 30 mL, greater than about 35 mL, greater than about 40 mL, greater than about 45 mL, greater than about 50 mL, greater than about 60 mL, greater than about 70 mL, greater than about 80 mL, greater than about 90 mL, greater than about 100 mL, greater than about 150 mL, greater than about 200 mL, greater than about 250 mL, greater than about 300 mL, greater than about 400 mL, greater than about 500 mL, greater than about 750 mL, greater than about 1 L, or greater than about 1.5 L. In some aspects of the device, the device body further comprises two openings, a distal opening, and a proximal opening. These opening may be the same radius, or they may be different radii. The radius of either the proximal or distal opening may, in some cases, be no more than about 1 mm, no more than about 2 mm, no more than about 3 mm, no more than about 4 mm, no more than about 5 mm, no more than about 10 mm, no more than about 15 mm, no more than about 20 mm, no more than about 25 mm, no more than about 30 mm, no more than about 35 mm, no more than about 40 mm, no more than about 45 mm, no more than about 50 mm, no more than about 60 mm. The radius of either the proximal or distal opening may, in some cases, be greater than about 1 mm, greater than about 2 mm, greater than about 3 mm, greater than about 4 mm, greater than about 5 mm, greater than about 10 mm, greater than about 15 mm, greater than about 20 mm, greater than about 25 mm, greater than about 30 mm, greater than about 35 mm, greater than about 40 mm, greater than about 45 mm, greater than about 50 mm, greater than about 60 mm. They may be, in some cases, circular, rectangular, or another shape. They may be designed to attached or reversibly couple to other devices. One opening may be designed to attached to one set of devices and the other opening designed to attach to a different set of devices. They may be designed to attached to a pipetman, pipetter, pipette tip, syringe, or fluid conducting cable, which may be of varying thickness, gauge, and material among other things. The device may have threading on the outer edged of the distal and proximal sides to facilitate attachment to other devices. The device may be designed to be physically integrated into automated laboratory preparation equipment. The device may be designed to be manipulated by these devices.

In some aspects of the device, it may be designed to enable liquid to flow through the body of the device. This fluid may flow from either the proximal side to the distal side or the reverse. During use of the device fluid may flow in multiple directions (e.g., from distal to proximal initially, then from proximal to distal for subsequent steps of enrichment). In some aspects of the method, the direction of the fluid flowing through the device may change once, twice, three times, or more throughout the execution of the method. Alternatively, use of the device may only use flow in a single direction. Fluid may flow through the device through the use of positive pressure, negative pressure, gravitational force, or passive diffusion among others. These types of fluid motivation may be used alone or in conjunction and may depend on the specific manifestation of the device.

### Chromatography Materials

Another aspect of the device comprises two or three different types of chromatography materials. These materials may be purchased from a common chemical supplier or may be made in the lab by the user. They may be a standard material or may be a custom-made unique material whether by a vendor or by the end users and not commercially available.

In some aspects of the device, the device may comprise a reverse phase chromatography material. In some cases, the device may have at least one, two, three, or more reverse phase chromatography material distinct layers. In some cases, the device may have at most one reverse phase chromatography material distinct layers. Reversed-phase chromatography is a technique using alkyl chains covalently bonded to the stationary phase particles in order to create a hydrophobic stationary phase, which has a stronger affinity for hydrophobic or less polar compounds. Reversed-phase chromatography employs a polar (aqueous) mobile phase. As a result, hydrophobic molecules in the polar mobile phase tend to adsorb to the hydrophobic stationary phase, and hydrophilic molecules in the polar mobile phase will pass through the column and are eluted first. Hydrophobic molecules may be eluted from the column by decreasing the polarity of the mobile phase using an organic (non-polar) solvent, which reduces hydrophobic interactions. The more hydrophobic the molecule, the more strongly it will bind to the hydrophobic stationary phase, and the higher the concentration of organic solvent that may be used to elute the molecule. Any inert polar substance that achieves sufficient packing may be used for reversed-phase chromatography. The non-polar stationary phase may be octadecyl carbon chain (C18)-bonded silica, a C8-bonded silica, C3-bonded silica, pure silica, cyano-bonded silica, underivatized polystyrene-divinyl benzene, or a phenyl-bonded silica. The device may use an octadecyl carbon chain (C18)-bonded silica, a C8-bonded silica, C3-bonded silica, pure silica, cyano-bonded silica, or a phenyl-bonded silica. In some aspects of the device the reverse phase silica may have different surface functionalization. This may be accomplished using a monomeric or a polymeric reaction with different short-chain organosilanes used in a second step to cover remaining silanol groups. The surface chemistries of different stationary phases may lead to changes in selectivity that will be better suited to different starting liquid and the glycopeptides that are undergoing enrichment. The size of the silica beads may be different sizes. The particle size of the beads may be based on the specifics of the separation. Larger bead size may provide larger capacities and potentially lesser pressures. Large-scale preparative processes may benefit by using beads of diameter, in some cases, greater than about 5 μm, greater than about 6 μm, greater than about 7 μm, greater than about 8 μm, greater than about 9 μm, greater than about 10 μm, greater than about 11 μm, greater than about 12 μm, greater than about 13 μm, greater than about 14 μm, greater than about 15 μm or larger. Large-scale preparative processes may benefit by using beads of diameter, in some cases, less than about 5 μm, less than about 6 μm, less than about 7 μm, less than about 8 μm, less than about 9 μm, less than about 10 μm, less than about 11 μm, less than about 12 μm, less than about 13 μm, less than about 14 μm, less than about 15 μm or smaller. Small-scale preparative and analytical separations may benefit with beads sizes no greater than about 10 μm, no greater than about 9 μm, no greater than about 8 μm, no greater than about 7 μm, no greater than about 6 μm, no greater than about 5 μm, no greater than about 4 μm, no greater than about 3 μm, no greater than about 2 μm, no greater than about 1 μm, no greater than about 0.5 μm, no greater than about 0.1 μm, or less. Small-scale preparative and analytical separations may benefit with beads sizes no less than about 10 μm, no less than about 9 μm, no less than about 8 μm, no less than about 7 μm, no less than about 6 μm, no less than about 5 μm, no less than about 4 μm, no less than about 3 μm, no less than about 2 μm, no less than about 1 μm, no less than about 0.5 μm, no less than about 0.1 μm, or more. The density of chromatography materials may be varied in order to better separate the desired fractions of each liquid or optimize the chromatography process.

In some aspects of the device, the first material is a reverse phase chromatography material.

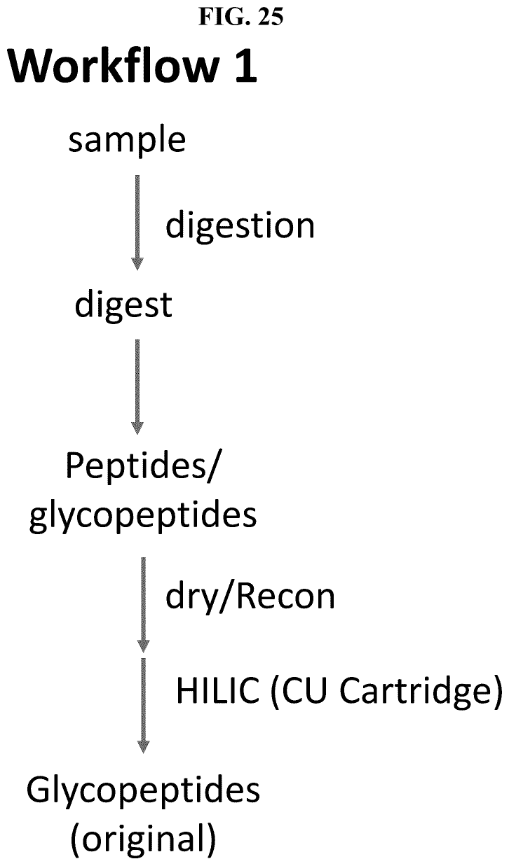

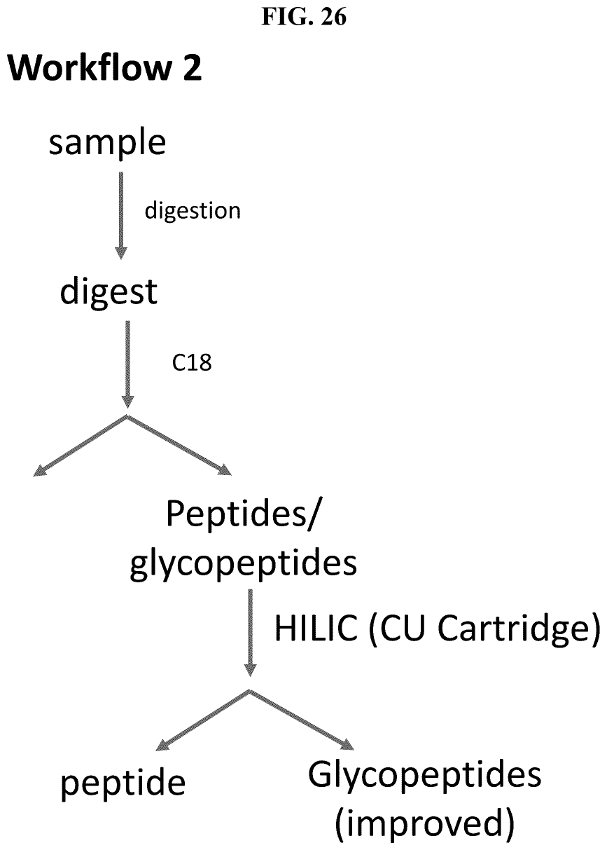

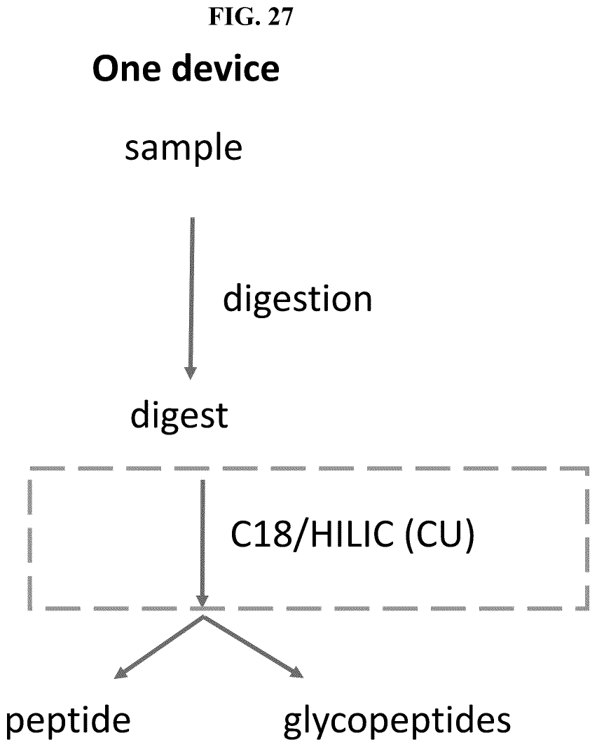

In another aspect of the device, the device may comprise a hydrophilic interaction liquid chromatography (HILIC) material. In some cases, the device may have at least one, two, three, or more hydrophilic interaction liquid chromatography (HILIC) material distinct layers. In some cases, the device may have at most one hydrophilic interaction liquid chromatography (HILIC) material distinct layer. HILIC is a variant of normal phase liquid chromatography that uses hydrophilic stationary phases with reversed-phase type mobile phases. In HILIC, the mobile phase forms a water-rich layer on the surface of the polar stationary phase vs. the water-deficient mobile phase, creating a liquid/liquid extraction system. The analyte is distributed between these two layers. However, HILIC is more than just simple partitioning and includes hydrogen donor interactions between neutral polar species as well as weak electrostatic mechanisms under the high organic solvent conditions used for retention. This distinguishes HILIC as a mechanism distinct from ion exchange chromatography. The more polar compounds will have a stronger interaction with the stationary aqueous layer than the less polar compounds. Thus, a separation based on a compound's polarity and degree of solvation takes place. HILIC mode of separation is used extensively for separation of some biomolecules, organic and some inorganic molecules due to its ability to separate out small organic acids, basic drugs, and many other neutral and charged substances. The characteristics of the hydrophilic stationary phase may affect and, in some cases, limit the choices of mobile phase composition, ion strength or buffer pH value available, since mechanisms other than hydrophilic partitioning could potentially occur. Any polar chromatographic surface may be used for HILIC separations. Typical HILIC stationary phases consist of classical bare silica or silica gels modified with many polar functional groups. The polar stationary phase may consist of classical bare silica or silica gels modified with many polar functional groups. Polymer-based stationary phases may be used. Modifications to the silica may be amino-silica, diol-silica, or amide-silica. Chemically bonded stationary phases with specific structural properties have been prepared. They may contain aminopropyl ligands bonded to silica, an alkylamide packing phase and or mixed phase containing different types of ligands (—NH2, —CN, -Ph, —C8, —C18) bonded to the support. In some embodiments, carbohydrates may be functionalized onto the HILIC stationary phase. It may comprise a zwitterionic substance bonded to the support. Alternatively, the hydrophilic interaction chromatography materials may be cellulose. The cellulose may be of a particular polymorph, density, or size. It may be microcrystalline cellulose. Many vendors sell both traditional HILIC materials and newer more sophisticated stationary phases. Novel separation materials for HILIC have attracted increasing attention in recent years thus the structural variations of HILIC-type stationary phases are wider than those found in reversed-phase systems. The device may use any HILIC chromatography material that is currently in use or may be developed. Workflow 1 (FIG. 25), workflow 2 (FIG. 26), and one device (FIG. 27), can use the polar stationary phases as described herein.

In another aspect of the device the second chromatography material comprises a hydrophilic interaction liquid chromatography (HILIC) material.

In another aspect of the device there may be a third chromatography material. This material may be the same material as used in the first reverse phase chromatography material. It may be a different reverse phase chromatography material. This chromatography material has the same disclosure as to the use and potential materials as the reverse phase chromatography material discussed above.

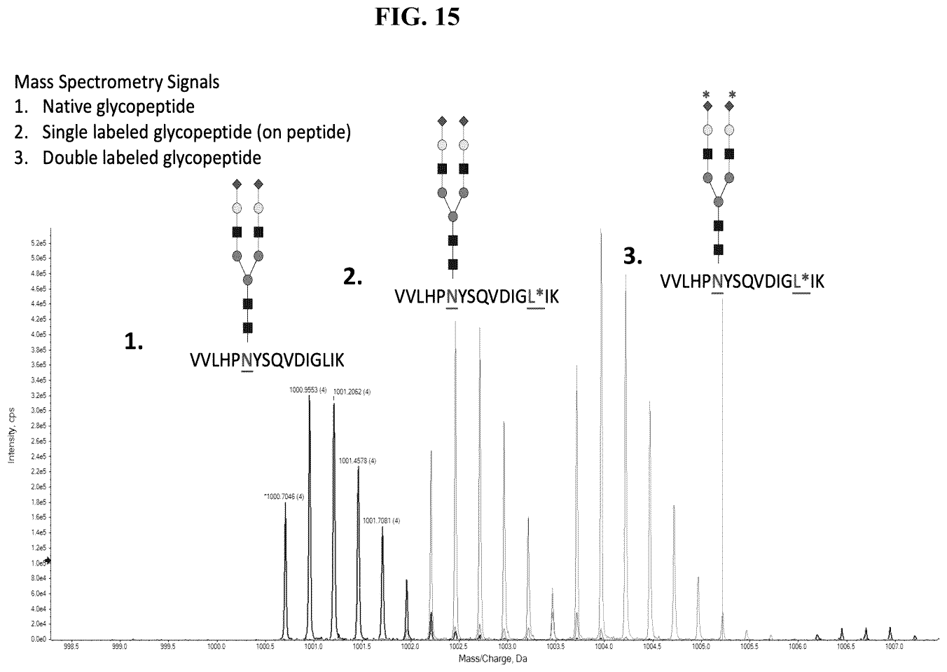

In another aspect of the device, the device may comprise a metal oxide material. The metal oxide material may be selected from TiO₂, SnO₂, Al₂O₃, Ga₂O₃, and Ta₂O₅. The metal oxide material may be TiO₂. In some aspects of the device, the metal oxide material may use the affinity interaction between the biofluid and the metal oxides to provide enriched phosphopeptides or phospholipids. The metal oxide material may come before a hydrophilic interaction chromatography material. The metal oxide material may come before a reverse phase chromatography material. The metal oxide material may be in the same device as the other materials or may be separate. As shown in FIG. 25 and FIG. 15, the metal oxide material is stacked on top of the reverse phase chromatography material, which is in turn stacked on a hydrophilic interaction chromatography material. The metal oxide material is useful for separating phosphopeptides from a biofluid. The metal oxide material is useful for separating phosphopeptides from glycopeptides. The metal oxide material is useful for removing phosphopeptides. Additionally, by removing these products at this stage, the lifetime of the following materials (e.g., C18 or HILIC material) can be extended. Further, the device may be useful for fractionating different post-translational modifications (PTMs) for in-depth proteomic analysis.

In another aspect of the device, each of the individual chromatography materials are homogeneous. Non-homogeneous chromatographic materials contain beads which have different modifications or functional groups mixed together. These serve to provide some separation between some peptide and glycopeptides. However, in this device the chromatographic material or each individual level will have identical compositions throughout the material. This will provide better separation of the peptides and glycopeptides as well as allowing for the isolation of certain fraction of the sample through a stepwise methodology elution.

### Device Kits and Assembly

In another aspect of the device, the two separate chromatography materials may be layered on top of each other within the body of the device such that two separate and distinct layers are formed within the device. These layers may be of different thicknesses and densities when compared to each other. In some aspects, the HILIC material may be above the stationary reverse phase chromatography material. In some aspects, the stationary reverse phase chromatography material may be above the HILIC material.

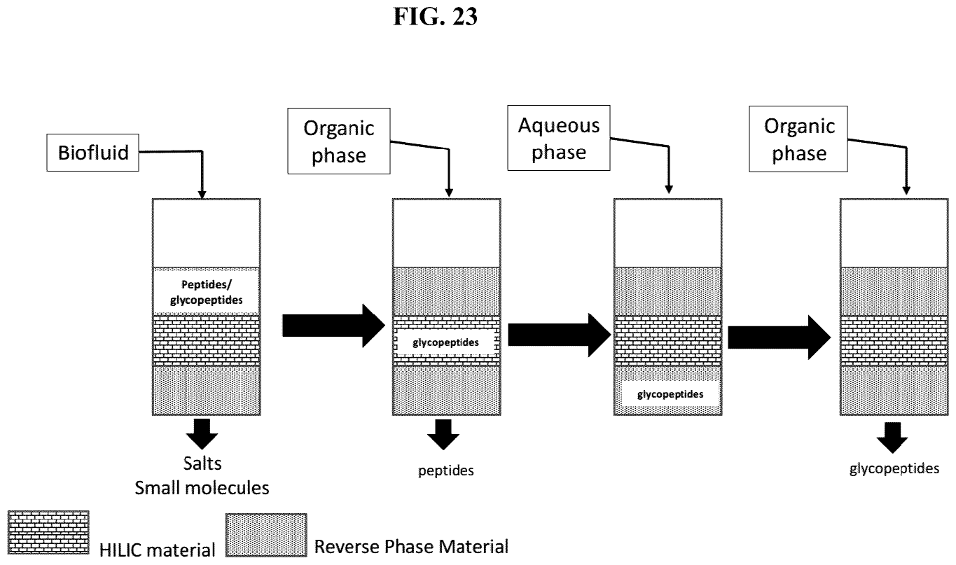

In another aspect of the device, the three separate chromatography materials may be layered on top of each other within the body of the device such that three separate and distinct layers are formed within the device. FIG. 23 displays one possible manifestation of the device as assembled with three distinct layers. These layers may be of different thicknesses and densities when compared to each other.

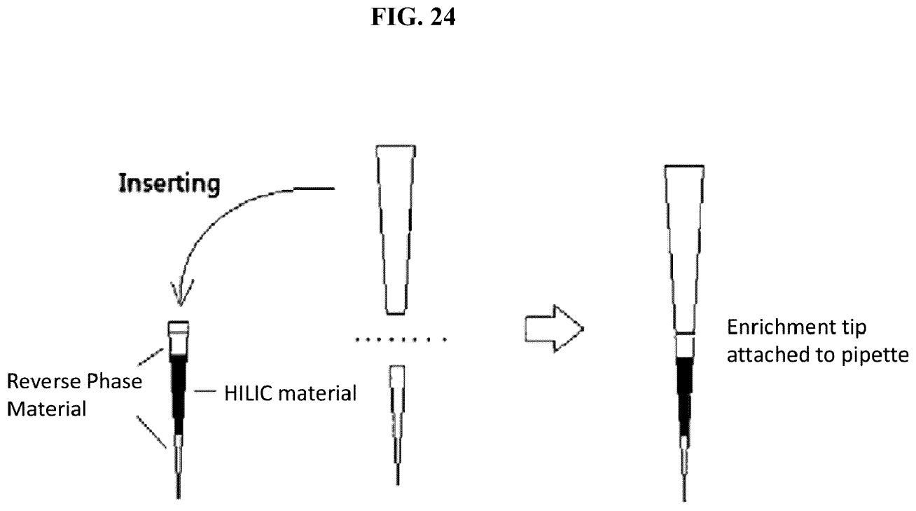

In some aspects, the layer of HILIC material will be between the two layers of reverse phase material. FIG. 24 displays one possible manifestation of the device as assembled with a pipette tip comprising the body of the device. This demonstrates the layering of the chromatography materials within the body of the device.

The materials may be packed such that the layers are fixed within the body of the device. This packing may allow for liquid to flow across the chromatography materials from either the distal or proximal opening of the device body without disturbing the packed material.

Another aspect of the device may provide a kit containing the materials that may be used to construct the disclosed device. The kit may provide a fully assembled device. It may alternatively provide the materials in a way that may allow user to assemble the device before use. The kit may not contain certain components that may allow the user to provide their own materials in order to customize the device to the specific needs of the user. The kit may further comprise instructions for the assembly and uses of the disclosed device.

### Method of Enriching Glycopeptides

Aspects of the method disclosed herein may enable one skilled in the art to the glycopeptides contained within a sample. The method may be performed with a device disclosed herein. The method may include various steps, such as sample collection, preparation, preliminary processing, storage, preservation, isolation, treatment, transformation, digestion, analysis, or identification. Some steps may include extra wash steps, multiple elution steps, or using a gradient elution. Method steps may be performed to better enrich the glycopeptides or be useful based on the nature of the glycopeptides, peptides, buffer, analytical procedures, chromatographic materials, or mobile phases among other things.

### Chromatographic Mobile Phases

Chromatography is a method by which a mixture is separated by distributing its components between two phases. The stationary phase remains fixed in place while the mobile phase carries the components of the mixture through the medium being used. The stationary phase acts as a constraint on many of the components in a mixture, slowing them down to move slower than the mobile phase. The movement of the components in the mobile phase is controlled by the significance of their interactions with the mobile and/or stationary phases. Because of the differences in factors such as the solubility of certain components in the mobile phase and the strength of their affinities for the stationary phase, some components will move faster than others, thus facilitating the separation of the components within that mixture. The mobile phase is sometimes referred to as the solvent, but the liquid used to suspend the sample may differ from the actual mobile phase.

In some aspects of the method, the enrichment is accomplished using at least one, two, three, four, five, six, seven, eight, nine, ten or more mobile phases. In some cases, the enrichment is accomplished using at least one, two, three, four, five, six, seven, eight, nine, ten, or more organic mobile phases. In some cases, the enrichment is accomplished using at least one, two, three, four, five, six, seven, eight, or more aqueous mobile phases. In some cases, the enrichment is accomplished using at most ten, nine, eight, seven, six, five, four, three, two, or fewer organic mobile phases. In some cases, the enrichment is accomplished using at most ten, nine, eight, seven, six, five, four, three, two, or fewer aqueous mobile phases. The different mobile phases may be used to elute the glycopeptides or other components of the biofluid at different times or at varying times or at similar times. The different mobile phases may pass through a single device containing the stationary phases. The different mobile phases may pass through a single device containing two stationary phases (e.g., one stationary reverse phase chromatography material and hydrophilic interaction chromatography material). The different mobile phases may pass through separate devices containing separate stationary phases.

In some aspects of the method, the enrichment is accomplished using at least one organic mobile phase and at least one aqueous mobile phase. This method may comprise applying a biofluid comprising the glycopeptides onto a hydrophilic interaction chromatography material. This method may comprise drying biofluids. This method may comprise reconstituting biofluids. The biofluids may be reconstituted using an organic mobile phase (e.g., 85% acetonitrile (ACN) with 1% trifluoroacetic acid (TFA)) before applying the biofluid to the hydrophilic interaction chromatography material. After applying the biofluid to the hydrophilic interaction chromatography material, the method may include washing the hydrophilic interaction chromatography material with an organic mobile phase (e.g., 85% ACN/1% TFA). Any bound glycopeptides may be eluted by an aqueous mobile phase (e.g., 0.1% formic acid).

In some aspects of the method, the enrichment is accomplished using at least one organic mobile phase and at least one aqueous mobile phase. The method may include using at least one organic mobile phase on a stationary reverse phase chromatography material. The method may then include using at least one aqueous mobile phase on a stationary hydrophilic interaction chromatography material. The hydrophilic interaction chromatography material may be subjected to at least one organic mobile phase. The stationary hydrophilic interaction chromatography material and the stationary reverse phase chromatography material may be separate from each other. In some aspects of the method, the method may include washing the stationary reverse phase chromatography material with an organic mobile phase (e.g., 85% ACN/1% TFA). The method may include equilibrating the stationary reverse phase chromatography material with an aqueous mobile phase (e.g., 1% TFA). The method may include washing the stationary reverse phase chromatography material with an aqueous mobile phase (e.g., 1% TFA). The method may include washing the stationary reverse phase chromatography material with an organic mobile phase (e.g., 85% ACN/1% TFA) to elute any bound peptides. The eluted bound peptides may be loaded onto the hydrophilic interaction chromatography material. The method may include priming the hydrophilic interaction chromatography material. The hydrophilic interaction chromatography material may be equilibrated with an organic mobile phase (e.g., 85% ACN/1% TFA). The hydrophilic interaction chromatography material may be washed with an organic mobile phase (e.g., 85% ACN/1% TFA). Any bound glycopeptides may then be eluted using an aqueous mobile phase (e.g., 0.1% formic acid).

In some aspects of the method, the enrichment is accomplished using at least one organic mobile phase and at least one aqueous mobile phase in a single device. The method may include using an organic mobile phase to remove the other peptides. The method may include using an organic mobile phase to separate the other peptides from the glycopeptides. The method may include using an aqueous mobile phase to remove the glycopeptides. The method may include washing the two materials of the device with an organic mobile phase (e.g., 85% ACN/1% TFA). The method may include equilibrating the two materials of the device with an aqueous mobile phase (e.g., 1% TFA). The method may include washing the device after adding the biofluid. The method may include washing the device after adding the biofluid using an aqueous mobile phase (e.g., 1% TFA). The method may include washing the device after adding the biofluid using an aqueous mobile phase (e.g., 1% TFA) more than once (e.g., twice). The peptides from the biofluid may then eluted from the device using an organic mobile phase (e.g., 85% ACN/1% TFA). The glycopeptides from the biofluid may then be eluted from the device using an aqueous mobile phase (e.g., 2% ACN/0.1% formic acid).

In some aspects of the method, the mobile phase may be an organic mobile phase. The organic mobile phase may include a solvent selected from acetonitrile, methanol, tetrahydrofuran, ethyl acetate, ethanol, isopropanol, chloroform, cyclohexane, hexane, acetone, butanol, carbon tetrachloride, cyclopentane, dichloroethane, dichloromethane, diethyl ether, dimethyl sulfoxide, dipropyl ether, dioxane, methyl ethyl ketone, octane, pentane, tetrachloroethane, toluene, trichloroethane, xylene, dimethylformamide, heptane, benzene, and dimethylacetamide. The organic mobile phase may also include water. The water of the organic mobile phase may be at a lower percentage (v/v) than the solvent of the organic mobile phase. The water of the organic mobile phase may be at least about 1% (v/v), at least about 5% (v/v), at least about 10% (v/v), at least about 15% (v/v), at least about 20% (v/v), at least about 25% (v/v), at least about 30% (v/v), at least about 35% (v/v), at least about 40% (v/v), or at least about 45% (v/v). The water of the organic mobile phase may be at most about 1% (v/v), at most about 5% (v/v), at most about 10% (v/v), at most about 15% (v/v), at most about 20% (v/v), at most about 25% (v/v), at most about 30% (v/v), at most about 35% (v/v), at most about 40% (v/v), or at most about 45% (v/v). The solvent selected for the organic mobile phase may be at least about 55% (v/v), at least about 60% (v/v), at least about 65% (v/v), at least about 70% (v/v), at least about 75% (v/v), at least about 80% (v/v), at least about 85% (v/v), at least about 90% (v/v), at least about 95% (v/v), at least about 97% (v/v), or at least about 99% (v/v). The solvent selected for the organic mobile phase may be at most about 55% (v/v), at most about 60% (v/v), at most about 65% (v/v), at most about 70% (v/v), at most about 75% (v/v), at most about 80% (v/v), at most about 85% (v/v), at most about 90% (v/v), at most about 95% (v/v), at most about 97% (v/v), or at most about 99% (v/v). The percentage of acetonitrile in an organic mobile phase may be at least about 55% (v/v), at least about 60% (v/v), at least about 65% (v/v), at least about 70% (v/v), at least about 75% (v/v), at least about 80% (v/v), at least about 85% (v/v), at least about 90% (v/v), at least about 95% (v/v), at least about 97% (v/v), or at least about 99% (v/v). The percentage of acetonitrile in an organic mobile phase may be at most about 55% (v/v), at most about 60% (v/v), at most about 65% (v/v), at most about 70% (v/v), at most about 75% (v/v), at most about 80% (v/v), at most about 85% (v/v), at most about 90% (v/v), at most about 95% (v/v), at most about 97% (v/v), or at most about 99% (v/v). In some embodiments, one or more additives may be added to the organic mobile phase. In some embodiments, the one or more additives may be selected from formic acid, acetic acid, trifluoroacetic acid, trichloroacetic acid, phosphoric acid, citric acid, propionic acid, carbonic acid, tris(hydroxymethyl)aminomethane, boric acid, ammonia, glycine, carbonic acid, pyrrolidine, phosphoric acid, dichloroacetic acid, chloroacetic acid, ammonium acetate, triethylamine, and ammonium hydroxide. The percentage of one or more additives included in the organic mobile phase may be at least about 0.1% (v/v), at least about 0.5% (v/v), at least about 1% (v/v), at least about 1.5% (v/v), at least about 1.75% (v/v), at least about 2% (v/v), at least about 3% (v/v), at least about 4% (v/v), at least about 5% (v/v), at least about 6% (v/v), at least about 7% (v/v), at least about 8% (v/v), at least about 9% (v/v), or at least about 10% (v/v). The percentage of one or more additives included in the organic mobile phase may be at most about 0.1% (v/v), at most about 0.5% (v/v), at most about 1% (v/v), at most about 1.5% (v/v), at most about 1.75% (v/v), at most about 2% (v/v), at most about 3% (v/v), at most about 4% (v/v), at most about 5% (v/v), at most about 6% (v/v), at most about 7% (v/v), at most about 8% (v/v), at most about 9% (v/v), or at most about 10% (v/v). The percentage of trifluoroacetic acid included in the organic mobile phase may be at least about 0.1% (v/v), at least about 0.5% (v/v), at least about 1% (v/v), at least about 1.5% (v/v), at least about 1.75% (v/v), at least about 2% (v/v), at least about 3% (v/v), at least about 4% (v/v), or at least about 5% (v/v). The percentage of trifluoroacetic acid included in the organic mobile phase may be at most about 0.1% (v/v), at most about 0.5% (v/v), at most about 1% (v/v), at most about 1.5% (v/v), at most about 1.75% (v/v), at most about 2% (v/v), at most about 3% (v/v), at most about 4% (v/v), or at most about 5% (v/v). The percentage of formic acid included in the organic mobile phase may be at least about 0.1% (v/v), at least about 0.5% (v/v), at least about 1% (v/v), at least about 1.5% (v/v), at least about 1.75% (v/v), at least about 2% (v/v), at least about 3% (v/v), at least about 4% (v/v), or at least about 5% (v/v). The percentage of formic acid included in the organic mobile phase may be at most about 0.1% (v/v), at most about 0.5% (v/v), at most about 1% (v/v), at most about 1.5% (v/v), at most about 1.75% (v/v), at most about 2% (v/v), at most about 3% (v/v), at most about 4% (v/v), or at most about 5% (v/v). The methods may use the organic mobile phase as described herein. Workflow 1, workflow 2, and one device may use the organic mobile phase as described herein.

In some aspects of the method, the mobile phase may be an aqueous mobile phase. The aqueous mobile phase includes water. The water of the aqueous mobile phase may be at a higher percentage (v/v) than the solvent of the aqueous mobile phase. The aqueous mobile phase may include a solvent selected from acetonitrile, methanol, tetrahydrofuran, ethyl acetate, ethanol, isopropanol, chloroform, cyclohexane, hexane, acetone, butanol, carbon tetrachloride, cyclopentane, dichloroethane, dichloromethane, diethyl ether, dimethyl sulfoxide, dipropyl ether, dioxane, methyl ethyl ketone, octane, pentane, tetrachloroethane, toluene, trichloroethane, xylene, dimethylformamide, heptane, benzene, and dimethylacetamide. The water for the aqueous mobile phase may be at least about 55% (v/v), at least about 60% (v/v), at least about 65% (v/v), at least about 70% (v/v), at least about 75% (v/v), at least about 80% (v/v), at least about 85% (v/v), at least about 90% (v/v), at least about 95% (v/v), at least about 97% (v/v), or at least about 99% (v/v). The water for the aqueous mobile phase may be at most about 55% (v/v), at most about 60% (v/v), at most about 65% (v/v), at most about 70% (v/v), at most about 75% (v/v), at most about 80% (v/v), at most about 85% (v/v), at most about 90% (v/v), at most about 95% (v/v), at most about 97% (v/v), or at most about 99% (v/v). The solvent of the aqueous mobile phase may be at least about 1% (v/v), at least about 5% (v/v), at least about 10% (v/v), at least about 15% (v/v), at least about 20% (v/v), at least about 25% (v/v), at least about 30% (v/v), at least about 35% (v/v), at least about 40% (v/v), or at least about 45% (v/v). The solvent of the aqueous mobile phase may be at most about 1% (v/v), at most about 5% (v/v), at most about 10% (v/v), at most about 15% (v/v), at most about 20% (v/v), at most about 25% (v/v), at most about 30% (v/v), at most about 35% (v/v), at most about 40% (v/v), or at most about 45% (v/v). The percentage of acetonitrile in an aqueous mobile phase may be at least about 1% (v/v), at least about 5% (v/v), at least about 10% (v/v), at least about 15% (v/v), at least about 20% (v/v), at least about 25% (v/v), at least about 30% (v/v), at least about 35% (v/v), at least about 40% (v/v), or at least about 45% (v/v). The percentage of acetonitrile in an aqueous mobile phase may be at most about 1% (v/v), at most about 5% (v/v), at most about 10% (v/v), at most about 15% (v/v), at most about 20% (v/v), at most about 25% (v/v), at most about 30% (v/v), at most about 35% (v/v), at most about 40% (v/v), or at most about 45% (v/v). In some embodiments, one or more additives may be added to the aqueous mobile phase. In some embodiments, the one or more additives may be selected from formic acid, acetic acid, trifluoroacetic acid, trichloroacetic acid, phosphoric acid, citric acid, propionic acid, carbonic acid, tris(hydroxymethyl)aminomethane, boric acid, ammonia, glycine, carbonic acid, pyrrolidine, phosphoric acid, dichloroacetic acid, chloroacetic acid, ammonium acetate, triethylamine, and ammonium hydroxide. The one or more additives may include a buffer. The percentage of one or more additives included in the aqueous mobile phase may be at least about 0.1% (v/v), at least about 0.5% (v/v), at least about 1% (v/v), at least about 1.5% (v/v), at least about 1.75% (v/v), at least about 2% (v/v), at least about 3% (v/v), at least about 4% (v/v), at least about 5% (v/v), at least about 6% (v/v), at least about 7% (v/v), at least about 8% (v/v), at least about 9% (v/v), or at least about 10% (v/v). The percentage of one or more additives included in the aqueous mobile phase may be at most about 0.1% (v/v), at most about 0.5% (v/v), at most about 1% (v/v), at most about 1.5% (v/v), at most about 1.75% (v/v), at most about 2% (v/v), at most about 3% (v/v), at most about 4% (v/v), at most about 5% (v/v), at most about 6% (v/v), at most about 7% (v/v), at most about 8% (v/v), at most about 9% (v/v), or at most about 10% (v/v). The percentage of trifluoroacetic acid included in the aqueous mobile phase may be at least about 0.1% (v/v), at least about 0.5% (v/v), at least about 1% (v/v), at least about 1.5% (v/v), at least about 1.75% (v/v), at least about 2% (v/v), at least about 3% (v/v), at least about 4% (v/v), or at least about 5% (v/v). The percentage of trifluoroacetic acid included in the aqueous mobile phase may be at most about 0.1% (v/v), at most about 0.5% (v/v), at most about 1% (v/v), at most about 1.5% (v/v), at most about 1.75% (v/v), at most about 2% (v/v), at most about 3% (v/v), at most about 4% (v/v), or at most about 5% (v/v). The percentage of formic acid included in the aqueous mobile phase may be at least about 0.10% (v/v), at least about 0.5% (v/v), at least about 1% (v/v), at least about 1.5% (v/v), at least about 1.75% (v/v), at least about 2% (v/v), at least about 3% (v/v), at least about 4% (v/v), or at least about 5% (v/v). The percentage of formic acid included in the aqueous mobile phase may be at most about 0.1% (v/v), at most about 0.5% (v/v), at most about 1% (v/v), at most about 1.5% (v/v), at most about 1.75% (v/v), at most about 2% (v/v), at most about 3% (v/v), at most about 4% (v/v), or at most about 5% (v/v). The methods may use the aqueous mobile phase as described herein. Workflow 1, workflow 2, and one device may use the aqueous mobile phase as described herein.

The organic mobile phase may be used to wet the stationary phase. The organic mobile phase may be used to solvate the stationary phase. The organic mobile phase may be used to prime the stationary phase. The organic mobile phase may be used to equilibrate the stationary phase. The organic mobile phase may be used to elute the glycopeptides from the stationary phase. The organic mobile phase may be flowed through the stationary phase automatically or manually. The organic mobile phase may be flowed through the stationary phase 1, 2, 3, 4, 5, 6, 7, 8, 9, 10, 11, 12, 13, 14, 15, 16, 17, 18, 19, 20, or more times. The organic mobile phase may be flowed through the stationary phase with a volume of at least about 1 microliter (μL), at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 110 μL, at least about 120 μL, at least about 130 μL, at least about 140 μL, at least about 150 μL, at least about 160 μL, at least about 170 μL, at least about 180 μL, at least about 190 μL, at least about 200 μL, at least about 500 μL, at least about 1000 μL, or more. The organic mobile phase may be flowed through the stationary phase with a volume of at most about 100 milliliters (mL), at most about 90 mL, at most about 80 mL, at most about 70 mL, at most about 60 mL, at most about 50 mL, at most about 40 mL, at most about 30 mL, at most about 20 mL, at most about 10 mL, at most about 9 mL, at most about 8 mL, at most about 7 mL, at most about 6 mL, at most about 5 mL, at most about 4 mL, at most about 3 mL, at most about 2 mL, at most about 1 mL, at most about 900 μL, at most about 800 μL, at most about 700 μL, at most about 600 μL, at most about 500 μL, at most about 400 μL, at most about 300 μL, at most about 200 μL, at most about 100 μL, at most about 90 μL, at most about 80 μL, at most about 70 μL, at most about 60 μL, at most about 50 μL, or less.

The aqueous mobile phase may be used to wet the stationary phase. The aqueous mobile phase may be used to solvate the stationary phase. The aqueous mobile phase may be used to prime the stationary phase. The aqueous mobile phase may be used to equilibrate the stationary phase. The aqueous mobile phase may be used to elute the glycopeptides from the stationary phase. The aqueous mobile phase may be flowed through the stationary phase automatically or manually. The aqueous mobile phase may be flowed through the stationary phase 1, 2, 3, 4, 5, 6, 7, 8, 9, 10, 11, 12, 13, 14, 15, 16, 17, 18, 19, 20, or more times. The aqueous mobile phase may be flowed through the stationary phase with a volume of at least about 1 microliter (μL), at least about 10 μL, at least about 20 μL, at least about 30 μL, at least about 40 μL, at least about 50 μL, at least about 60 μL, at least about 70 μL, at least about 80 μL, at least about 90 μL, at least about 100 μL, at least about 110 μL, at least about 120 μL, at least about 130 μL, at least about 140 μL, at least about 150 μL, at least about 160 μL, at least about 170 μL, at least about 180 μL, at least about 190 L, at least about 200 μL, at least about 500 μL, at least about 1000 μL, or more. The aqueous mobile phase may be flowed through the stationary phase with a volume of at most about 100 milliliters (mL), at most about 90 mL, at most about 80 mL, at most about 70 mL, at most about 60 mL, at most about 50 mL, at most about 40 mL, at most about 30 mL, at most about 20 mL, at most about 10 mL, at most about 9 mL, at most about 8 mL, at most about 7 mL, at most about 6 mL, at most about 5 mL, at most about 4 mL, at most about 3 mL, at most about 2 mL, at most about 1 mL, at most about 900 μL, at most about 800 μL, at most about 700 μL, at most about 600 μL, at most about 500 μL, at most about 400 μL, at most about 300 L, at most about 200 μL, at most about 100 μL, at most about 90 μL, at most about 80 μL, at most about 70 μL, at most about 60 μL, at most about 50 μL, or less.

In some aspects of the method, the enrichment is accomplished using three mobile phases, two organic mobile phase and an aqueous mobile phase. The organic mobile phase may interrupt the binding of the glycopeptides to the reverse phase chromatography material. While the aqueous mobile phase may interrupt the binding of the glycopeptide to the HILIC chromatography material. The organic phase may allow for the elution of peptides from the reverse phase chromatography material by interfering with the binding material with peptides while not allowing interactions to occur between the HILIC material and the peptides.

In some aspects of the method the organic phase may be different for different glycopeptides, sample types, and chromatography materials. It may be chosen with respect to the effect it may have on column pressure, its absorption spectrum, elution strength, separation selectivity, retention behavior, potential precipitation from mixing with certain buffers, or heat of reaction from mixing with water. The organic phase used to elute the glycopeptides from the first layer of reverse phase chromatography material may be the same or different from the organic mobile phase used to elute the glycopeptide from the second reverse phase chromatography material. A range of organic solvents are suitable for use as the organic mobile phase in chromatography, although in practice, only a few have been used routinely. In some aspects of the method, the organic mobile phase may be chosen from tetrahydrofuran, acetone, isopropanol, acetonitrile, or methanol. The mobile phase may further comprise water. The percentage of water (v/v) in the mobile phase may, in some cases, be no more than about 1%, no more than about 5%, no more than about 10%, no more than about 15%, no more than about 20%, no more than about 25%, no more than about 30%, no more than about 35%, no more than about 40%, no more than about 45%, no more than about 50%, no more than about 55%, no more than about 60%, or less. The percentage of water (v/v) in the mobile phase may, in some cases, be no less than about 1%, no less than about 5%, no less than about 10%, no less than about 15%, no less than about 20%, no less than about 25%, no less than about 30%, no less than about 35%, no less than about 40%, no less than about 45%, no less than about 50%, no less than about 55%, no less than about 60%, or more.

In some aspects of the method the aqueous mobile phase may be water. However, reverse phase chromatography materials should usually be operated with at least 5% organic modifier, unless otherwise stated. Under 95 to 100% aqueous conditions, the columns are likely to undergo dewetting or phase collapse which means the mobile phase will be expelled from the porous system due to surface tension. The percentage of water in the aqueous phase of the method may, in some cases, be no more than about 95%, no more than about 90%, no more than about 85%, no more than about 80%, no more than about 75%, no more than about 70%, no more than about 65%, no more than about 60%, no more than about 55%, no more than about 50%, no more than about 45%, no more than about 40%, no more than about 35% or more. The percentage of water in the aqueous phase of the method may, in some cases, be no less than about 95%, no less than about 90%, no less than about 85%, no less than about 80%, no less than about 75%, no less than about 70%, no less than about 65%, no less than about 60%, no less than about 55%, no less than about 50%, no less than about 45%, no less than about 40%, no less than about 35% or less. The organic modifier may be any one of the organic solvents discussed above as the organic mobile phase. The modifier may be the same as the mobile organic phase solvent, but it does not necessarily need to be the same.

In some aspects of the method, both the organic and aqueous mobile phase may include an acid in order to adjust the pH of the mobile phase. Analyte retention in reverse-phase is dictated by analyte hydrophobicity. For ionizable analytes, as the degree of ionization increases, retention typically decreases providing that no alternative modes of interaction such as ion exchange are present. The mobile phase pH determines the ionization state of ionizable analytes. The mobile phase pH may therefore be varied and used as a powerful tool to control analyte retention, peak shape, and selectivity. With reversed phase chromatography, this may be accomplished by adding formic, acetic, or trifluoroacetic acid to each solvent. With HILIC, this may be accomplished by adding formic, acetic, or trifluoroacetic acid to each solvent. The acid selected for the organic and aqueous mobile phases may be the same or may be different. The acid content of the mobile phases may, in some cases, be no more than about 0.01% (v/v), no more than about 0.05%, no more than about 0.1%, no more than about 0.15%, no more than about 0.2%, no more than about 0.3%, no more than about 0.4%, no more than about 0.5%, no more than about 0.6%, no more than about 0.7%, no more than about 0.8%, no more than about 0.9% no more than about 1.0%, no more than about 1.25%, no more than about 1.5%, no more than about 1.75%, no more than about 2.0, no more than about 2.25%, no more than about 2.5%, no more than about 2.75%, no more than about 3.0%, no more than about 3.5%, no more than about 4.0%, no more than about 4.5%, no more than about 5.0%, or more. The acid content of the mobile phases may, in some cases, be no less than about 0.01% (v/v), no less than about 0.05%, no less than about 0.1%, no less than about 0.15%, no less than about 0.2%, no less than about 0.3%, no less than about 0.4%, no less than about 0.5%, no less than about 0.6%, no less than about 0.7%, no less than about 0.8%, no less than about 0.9% no less than about 1.0%, no less than about 1.25%, no less than about 1.5%, no less than about 1.75%, no less than about 2.0%, no less than about 2.25%, no less than about 2.5%, no less than about 2.75%, no less than about 3.0%, no less than about 3.5%, no less than about 4.0%, no less than about 4.5%, no less than about 5.0%, or less.

In some aspects of the method the flow of the mobile phase through the chromatography materials may be changed linearly using two or more pumps. The two or more mobile phases may be prepared and a program of linearly adjusting the percentage of each phase over time may be programed into the pumps through a controller. This linear gradient flow gradient may allow for separation of peptides and glycopeptides from other peptides and glycopeptides respectively. It may allow for greater purity or enrichment of the desired fraction of glycopeptides. This method may be limited to certain aspects for the disclosed device in order to allow for this aspect of the method. Alternatively in some aspects of the method, a step-gradient is performed using a single source for each phase used in the method. This aspect of the method may use a set volume of the mobile phases or a set time and flow rate. Once the set point has been reached the next mobile phase in the method may be added onto the column with its own set point of either volume or time and flow rate. This may be continued with as many mobile phases as the method may use for the enrichment desired.

In some aspects of the method, the flow rate of the mobile phases through the device may be controlled. It may be set to a constant rate for the entire method, or it may be varied by during each step of the method. The flow rate may, in some cases, be no greater than about 0.1 mL/min, no greater than about 0.25 mL/min, no greater than about 0.5 mL/min, no greater than about 0.75 mL/min, no greater than about 1 mL/min, no greater than about 1.5 mL/min, no greater than about 2 mL/min, no greater than about 3 mL/min, no greater than about 4 mL/min, no greater than about 5 mL/min, no greater than about 10 mL/min, no greater than about 20 mL/min, no greater than about 30 mL/min, no greater than about 40 mL/min, no greater than about 50 mL/min, no greater than about 60 mL/min, no greater than about 70 mL/min, no greater than about 80 mL/min, no greater than about 90 mL/min, no greater than about 100 mL/min, no greater than about 125 mL/min, no greater than about 150 mL/min, no greater than about 175 mL/min, no greater than about 200 mL/min, no greater than about 250 mL/min, no greater than about 300 mL/min, no greater than about 350 mL/min, no greater than about 400 mL/min, no greater than about 500 mL/min or more. The flow rate may, in some cases, be no less than about 0.1 mL/min, no less than about 0.25 mL/min, no less than about 0.5 mL/min, no less than about 0.75 mL/min, no less than about 1 mL/min, no less than about 1.5 mL/min, no less than about 2 mL/min, no less than about 3 mL/min, no less than about 4 mL/min, no less than about 5 mL/min, no less than about 10 mL/min, no less than about 20 mL/min, no less than about 30 mL/min, no less than about 40 mL/min, no less than about 50 mL/min, no less than about 60 mL/min, no less than about 70 mL/min, no less than about 80 mL/min, no less than about 90 mL/min, no less than about 100 mL/min, no less than about 125 mL/min, no less than about 150 mL/min, no less than about 175 mL/min, no less than about 200 mL/min, no less than about 250 mL/min, no less than about 300 mL/min, no less than about 350 mL/min, no less than about 400 mL/min, no less than about 500 mL/min or less.

### Sample Types

In another aspect of the method, the sample used in the method as the source of the glycopeptide may be derived from a variety of sources. The sample may be a biofluid sample. Examples of biofluids include blood, serum, or plasma. Other examples of biofluids include urine, tears, semen, milk, vaginal fluid, mucus, saliva, or sweat. A biofluid may include a tissue or cell homogenate. A biofluid sample may be obtained from a subject. For example, a blood, serum, or plasma sample may be obtained from a subject by a blood draw. Other ways of obtaining biofluid samples include aspiration or swabbing. The biofluid sample may be cell-free or substantially cell-free. To obtain a cell-free or substantially cell-free biofluid sample, a biofluid may undergo a sample preparation method such as centrifugation and pellet removal.

In some aspects of the method, the sample may be acidified. The sample may be acidified using TFA. The sample may be acidified using acetic acid. The sample may be acidified using formic acid.

In some aspects of the method, a sample may be derived from in vitro experiments on cells. These cells may have undergone specific treatments such as the introduction of small molecules, proteins, peptides, nucleic acids, biomolecules, or other materials that may affect cellular function. The sample may be of the extracellular matrix, the cellular homogenate, or a fraction thereof.

A non-biofluid sample may be obtained from a subject. For example, a sample may include a tissue sample. The sample may include a cell sample. The sample may include a homogenate of a cell or tissue. The sample may include a supernatant of a centrifuged homogenate of a cell or tissue. Glycopeptides may be enriched from a single sample, or from multiple samples.

In another aspect of the method, the sample may be treated before the method for enrichment of the glycopeptide portion of the sample is performed. This treatment may be a protein digestion. Protein digestion, either enzymatically or nonenzymatically, is an important tool in protein identification, characterization, and quantification by proteomics strategies. The most widely applied method for protein digestion involves the use of enzymes. Many proteases are available for this purpose, each having their own characteristics in terms of specificity, efficiency, and optimum digestion conditions. The digestion may be accomplished using Arg-C, Asp-N, Glu-C, Lys-C, Lys-N, trypsin, chymotrypsin, pepsin, thermolysin, papain, or pronas. The digestion may be done through chemical cleavage. It may be achieved by treatment with dilute solutions of formic acid, hydrochloric acid, or acetic acid, or with other chemicals such as cyanogen bromide, 2-nitro-5-thiocyanobenzoate, or hydroxylamine. It may be accomplished using electrochemical oxidation which results in the nonenzymatic cleavage at Tyr and Trp. The digestion may be accomplished through a combination of any of the previous methods.

In some aspects of the method, the sample may further comprise chaotropes, surfactants, salts, and organic solvents. This may, in some cases, include phosphate buffered saline, acetate buffered saline, tris(hydroxymethyl)aminomethane, (4-(2-hydroxyethyl)-1-piperazineethanesulfonic acid), [tris(hydroxymethyl)methylamino]propanesulfonic acid, 2-(bis(2-hydroxyethyl)amino)acetic acid, N-[tris(hydroxymethyl)methyl]glycine, 3-[N-tris(hydroxymethyl)methylamino]-2-hydroxypropanesulfonic acid, 2-[[1,3-dihydroxy-2-(hydroxymethyl)propan-2-yl]amino]ethanesulfonic acid, 3-(N-morpholino)propanesulfonic acid, piperazine-N,N′-bis(2-ethanesulfonic acid), dimethylarsenic acid, 2-(N-morpholino)ethanesulfonic acid, sodium chloride, urea, tween, magnesium chloride, guanidinium chloride, methanol, dimethyl sulfoxide, acetonitrile, formamide, and sodium deoxycholate among others depending on the type of sample and the desired glycopeptide.

### Applications of Method or Device

In some aspects of the present disclosure, the invention may be used for the preparation of glycopeptides on a large scale. The size of the device, the flow rate of the mobile phases, and the amount of glycopeptide loaded onto the device may be scaled to accommodate larger batches of glycopeptides. The sample may be dried and reconstituted prior to loading onto the device. The sample may be suspended in an organic mobile phase. The sample may be suspended in an aqueous mobile phase. The amount of sample (e.g., biofluid, digest, plasma) loaded onto the device may, in some cases, be no more than about 1 mg, no more than about 5 mg, no more than about 10 mg, no more than about 15 mg, no more than about 20 mg, no more than about 25 mg, no more than about 50 mg, no more than about 100 mg, no more than about 200 mg, no more than about 300 mg, no more than about 400 mg, no more than about 500 mg, no more than about 600 mg, no more than about 700 mg, no more than about 800 mg, no more than about 900 mg, no more than about 1 g, no more than about 2 g, no more than about 3 g, no more than about 4 g, no more than about 5 g, no more than about 6 g, or more. The amount of sample loaded onto the device may be less than 0.1 mg. The amount of sample loaded onto the device may, in some cases, be no less than about 0.1 mg, no less than about 0.2 mg, no less than about 0.3 mg, no less than about 0.4 mg, no less than about 0.5 mg, no less than about 0.6 mg, no less than about 0.7 mg, no less than about 0.8 mg, no less than about 0.9 mg, no less than about 1 mg, no less than about 5 mg, no less than about 10 mg, no less than about 15 mg, no less than about 20 mg, no less than about 25 mg, no less than about 50 mg, no less than about 100 mg, no less than about 200 mg, no less than about 300 mg, no less than about 400 mg, no less than about 500 mg, no less than about 600 mg, no less than about 700 mg, no less than about 800 mg, no less than about 900 mg, no less than about 1 g, no less than about 2 g, no less than about 3 g, no less than about 4 g, no less than about 5 g, no less than about 6 g or less. The method may be customized in order to facilitate the enrichment of larger masses and/or faster enrichment of glycopeptides. This may be changes to the flow rate, mobile phase compositions, chromatography materials, and size of the device.

In another aspect of the present disclosure, the device or method may be integrated into an automated system. The system may be designed to perform preparation of sample for analysis, for analysis of prepared samples or both. The system may be designed to perform screening of chemical compounds to discover new medicines or other useful and desirable properties of chemicals, mapping enzyme networks in cells, mapping control networks of gene expression, elucidating mechanisms of signal transduction and determining the cellular constituents comprising signal transduction pathways, determining mechanisms of pathogenesis, such as the control of gene expression in bacteria when stimulated to form a biofilm, the host response to an etiologic agent, or other pathogenic activity, or determining other desirable knowledge of biological mechanisms that may be put to utility. The system may be of modular design and only implement the disclosed method when a workflow that may benefit from the enrichment of glycopeptides is activated. Code may be written in order to allow the system to follow the method. This code may enable the system to communicate with a controller that performs the method. The system may perform analysis of the enriched glycopeptide. This analysis may be UV/Vis spectroscopy, mass spectroscopy, glycopeptide identification, or glycopeptide profiling among others. In some aspects of the device, it may be in direct fluid communication with the analytical device. In some aspects, the elution from the device, or some fraction thereof, may flow directly into the ionization chamber of a mass spectrometry device.

### Generation of a Double-Labeled Glycopeptide Standard

Disclosed herein in some aspects are methods for generating a labeled glycopeptide standards (e.g., peptide-backbone- and glycan-modified standards). The double-labeled peptide standard may comprise a peptide. The double-labeled peptide standard may comprise a PTM. The PTM may comprise glycosylation. The PTM may comprise a glycan. The double-labeled peptide standard may comprise a peptide and a PTM. The double-labeled peptide standard may comprise enriched or purified peptides comprising the PTM. The double-labeled peptide standard may be generated from a portion of the biological sample comprising the enriched or purified peptide. The double-labeled peptide standard may be generated from a portion of the biological sample that is a majority of the biological sample. The double-labeled peptide standard may be generated from a portion of the biological sample that is e.g., about 90% or more of the biological sample. The double-labeled peptide standard may be generated from a portion of the biological sample that is half or less than half of the biological sample. The double-labeled peptide standard may be generated from a portion of the biological sample that is half or more than half of the biological sample.

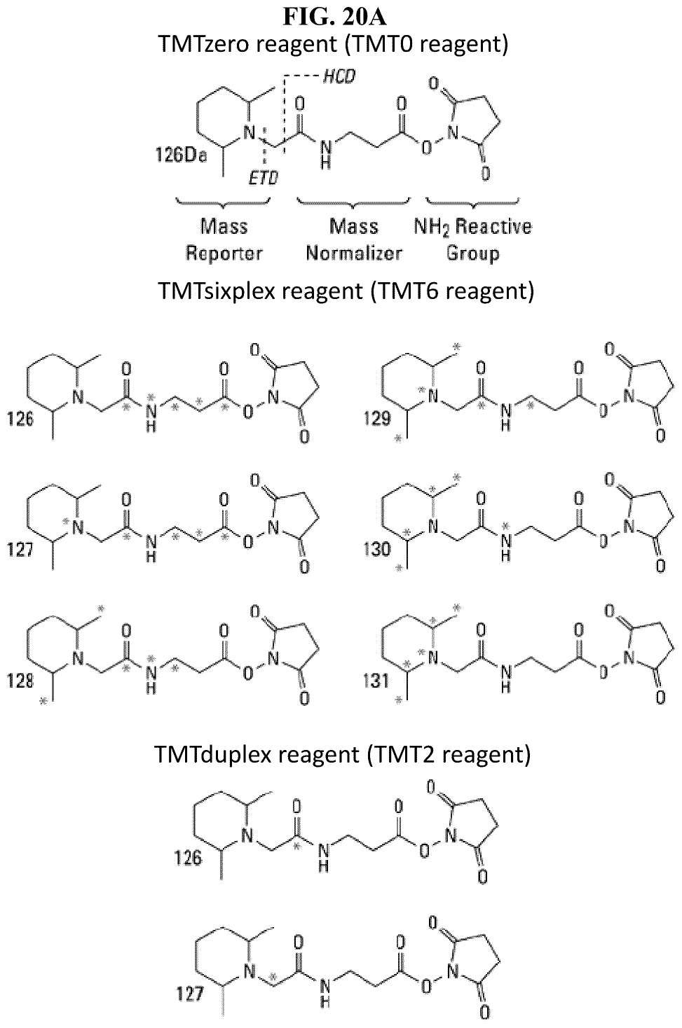

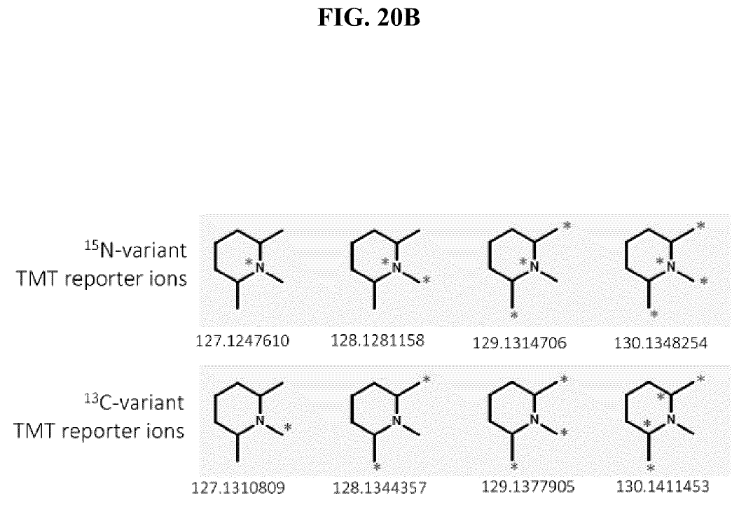

Generation of the double-labeled peptide standard may comprise adding a first label. Generation of the double-labeled peptide standard may comprise adding a first label to the enriched or purified portion of the biological sample. The first label may comprise a mass (e.g., isotopic) label. The offset label may comprise an isobaric label. The isobaric label may comprise TMT. The first label may comprise an offset label comprising the same chemical structure as isobaric tags that may be added to a portion of the biological sample that is not enriched or purified (e.g., the endogenous samples). In some aspects, the offset label may have higher molecular weight than the isobaric tags used in the endogenous samples. In some aspects, the offset label may have a higher molecular weight by incorporating more heavy isotope elements than the isobaric tags used in the endogenous samples. In some aspects, the higher molecular weight may help in distinguish the double-labeled peptide standard from the endogenous samples on a mass spectrum. In some aspects, the offset label may generate the double-labeled peptide standard to have similar chemical structure and physical properties as the endogenous samples. For example, in FIG. 1, a mass (e.g., isotopic) label may be added to the enriched glycopeptides. The mass (e.g., isotopic) label may be added linked to the peptide backbone of the enriched or purified portion of the biological sample. The mass label may comprise an isobaric label. The isobaric label may comprise a TMT. Isobaric tags as described herein can include any of the isobaric tags described in e.g., FIG. 20A, 20B, 21, or 22, or any combination thereof. The mass label may comprise the same mass as a peptide that the mass label is added to. The mass label may vary in terms of distribution of heavy isotopes in its structure. The mass label may comprise a mass reporter region.

Examples of additional isobaric labeling methods include isobaric tags for relative and absolute quantification (iTRAQ), mass differential tags for absolute and relative quantification, and DiLeu labeling. Isobaric tags as described herein can include any of the isobaric tags described in e.g., FIG. 20A, 20B, 21, or 22, or any combination thereof. The isobaric tag may comprise a mass reporter region, a linker region, a mass normalization region, a protein reactive group, or combinations thereof. The link region may be cleavable. The isobaric label may comprise amine-reactive tags. The isobaric label may react with cysteine residues. The isobaric label may react with carbonyl groups. The isobaric label may be used to help distinguish between the enriched or purified biological samples (e.g., the double-labeled peptide standard) from the endogenous samples. The isobaric labels may be used to distinguish the enriched or purified biological samples (e.g., the double-labeled peptide standard) from the endogenous samples on a mass spectrometer. The isobaric label may provide for multiplexing.

Generation of the double-labeled peptide standard may comprise adding a second label. Generation of the double-labeled peptide standard may comprise adding a second label to the enriched or purified portion of the biological sample. The second label may be an isotope label. For example, in FIG. 1, an isotope label is added linked to a glycan of the enriched glycopeptides. The isotope label may be added to the glycan PTM portion of the enriched or purified portion of the biological sample. The isotope label may be added to the glycan portion of the enriched or purified portion of the biological sample by enzymatic reaction.

In some cases, the second (e.g., glycan-linked) label can be added to a glycan chain of a glycopeptide or glycolipid described herein through the use of one or more enzyme described in Table 1 or Table 2 below.

TABLE 1
Example Glycosidases Useful with Methods According to the Disclosure

| | | | Example |
|---|---|---|---|
| Reaction | Enzyme | Example Species | Source |
| Removal of α2-3 | α2-3 Neuraminidase S | Streptococcus | New |
| linked sialic | | pneumoniae | England |
| acid | | | Biolabs |
| Removal of | α2-3,6,8 | Clostridium | New |
| sialic acid | Neuraminidase | perfringens | England |
| | | | Biolabs |
| Removal of | β1-4 Galactosidase S | Streptococcus | New |
| β1-4 linked | | pneumoniae | England |
| galactose | | | Biolabs |
| Removal of | β1-3 Galactosidase S | Xanthomonas | New |
| β1-3 linked | | manihotis | England |
| galactose | | | Biolabs |
| Removal of α1-2 | α1-2 Fucosidase | Xanthomonas | New |
| linked fucose | | manihotis | England |
| | | | Biolabs |
| Removal of | α1-3,4 Fucosidase | Prunus dulcis | New |
| α1-3/4 linked | | | England |
| fucose | | | Biolabs |
| Removal of | α1-2,3,6 Mannosidase | Canavalia | New |
| mannose | | ensiformis | England |
| | | | Biolabs |
| Removal of N- | β-N- | Streptococcus | New |
| Acetylglucosamine | Acetylglucosaminidase | pneumoniae | England |
| | S | | Biolabs |
| Removal of N- | α-N- | Chryseobacterium | New |
| Acetylgalactosamine | Acetyl- | meningosepticum | England |
| | galactosaminidase | | Biolabs |

TABLE 2
Example Glycosyltransferases (e.g., Glycosylases) Useful with
Methods According to the Disclosure

| | | Example | Example |
|---|---|---|---|
| Reaction | Enzyme | Species | source |
| α2-6 sialylation | α2-6 sialyltransferase | Homo | Millipore- |
| | 1 (ST6Gal1) | sapiens | Sigma |
| α2-3 sialylation | α2-3 sialyltransferase 3/4 | Homo | Recombinant |
| | (ST3Gal3, ST3Gal4) | sapiens | expression |
| β1-4 galactosylation | β1-4 Galacto- | Homo | Millipore- |
| | syltransferase 1 | sapiens | Sigma |
| | (β4GalT1) | | |
| β1-3 galactosylation | β1-3 Galacto- | Homo | Recombinant |
| | syltransferase 5 | sapiens | expression |
| | (β3GalT5) | | |
| α1-2 fucosylation | Fucosyltransferase | Homo | Recombinant |
| | 1 and 2 (FucT1 | sapiens | expression |
| | and FucT2) | | |
| α1-3 fucosylation | Fucosyltransferase | Homo | Recombinant |
| | 4 and 7 (FucT4 | sapiens | expression |
| | and FucT7) | | |
| α1-4 fucosylation | Fucosyltransferase | Homo | Recombinant |
| | 3 (FucT3) | sapiens | expression |
| β1-3 N- | β1-3N- | Homo | Recombinant |
| acetylgluco- | acetylglucosa- | sapiens | expression |
| saminylation | minyltransferase 2 | | |
| | (B3GNT2) | | |
| β1-6 N- | β1-6 N-acetylgluco- | Homo | Recombinant |
| acetylgluco- | saminylation | sapiens | expression |
| saminylation | (GCNT2) | | |

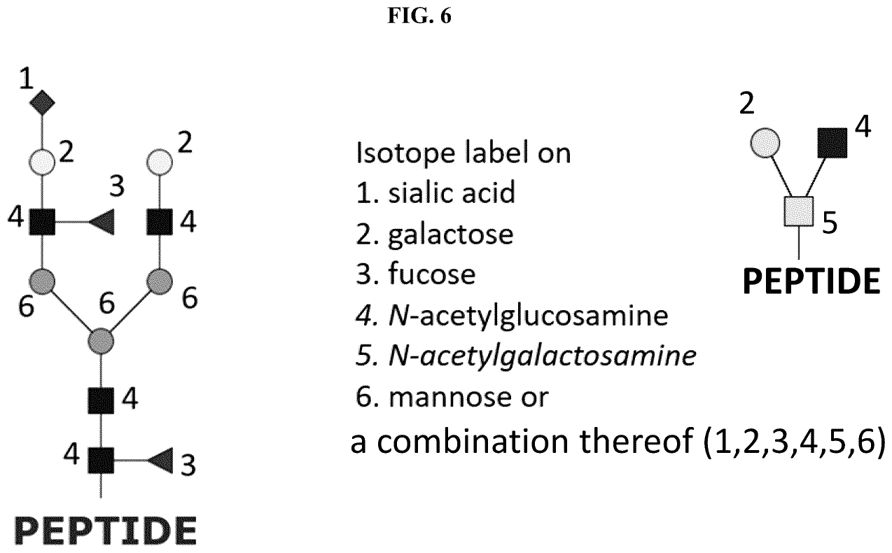

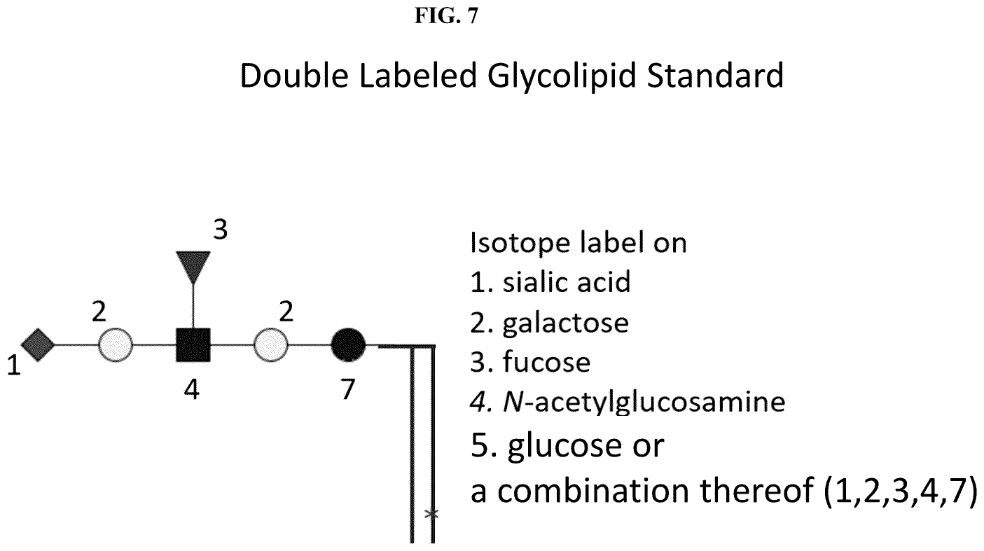

The second (e.g., glycan-linked, optionally isotopic) label may be added a glycan chain of the enriched or purified portion of the biological sample by one enzyme. The isotope label may be added to the glycan portion of the enriched or purified portion of the biological sample by more than one enzyme, more than two enzymes, more than three enzymes, more than four enzymes, more than five enzymes, more than six enzymes, more than seven enzymes, more than eight enzymes, more than nine enzymes, or more than ten enzymes. The isotope label may be added to the glycan portion of the enriched or purified portion of the biological sample by more than 10 enzymes, more than 20 enzymes, more than 30 enzymes, more than 40 enzymes, or more than 50 enzymes. The enzyme may trim a monosaccharide residue from a native glycan of the enriched or purified portion of the biological sample. The enzyme may trim more than one, more than two, more than three, more than four, more than five, more than six, more than seven, more than eight, more than nine, or more than ten monosaccharide residues from the native glycan of the enriched or purified portion of the biological sample. The enzyme may add a labeled monosaccharide residue on the enriched or purified portion of the biological sample. The enzyme may add more than one, more than two, more than three, more than four, more than five, more than six, more than seven, more than eight, more than nine, or more than ten labeled monosaccharide residues on the enriched or purified portion of the biological sample. Examples of monosaccharides include glucose, galactose, mannose, N-acetylglucoasmine, N-acetylgalactosamine, sialic acid, or combinations thereof (as shown in FIG. 6 and FIG. 7). The sialic acid may comprise Neu5Ac, Neu5Gc, KDN, or combinations thereof. The monosaccharide residue from the native glycan and the labeled monosaccharide may be the same. The monosaccharide residue from the native glycan and the labeled monosaccharide residue may be different. The monosaccharide residue from the native glycan and the labeled monosaccharide residue may have the same mass. The monosaccharide residue from the native glycan and the labeled monosaccharide residue may have different masses. The glycan-linked isotopic label can be specific for: (i) a disaccharide linkage type of the enriched glycan-modified macromolecules, or (ii) a monosaccharide of the glycan of the enriched glycan-modified macromolecules. The glycan-linked isotopic label can comprise a naturally-occurring monosaccharide comprising at least one, two, three, four, five, six, or more heavy isotope atoms.

Installing the second (e.g., glycan-linked, optionally isotopic) label may involve one or more enzymatic reaction using e.g., at least one enzyme described in Table 1 or Table 2. In some cases, the installation can involve treating a plurality of enriched glycan-modified macromolecules with a monosaccharide-specific or linkage-specific glycosidase to remove a predetermined monosaccharide from a glycan of the plurality of enriched glycan-modified macromolecules. In some embodiments, the installation can comprise treating the plurality of enriched glycan-modified macromolecules with a linkage-specific glycosidase, wherein the linkage-specific glycosidase comprises a glycosidase specific for an α2-3 sialic acid linkage, a β1-4 galactose linkage, a β1-3 galactose linkage, an α1-2 fucose linkage, or an α1-3/4 fucose linkage. In some embodiments, the linkage-specific glycosidase comprises α2-3 Neuraminidase S, β1-4 Galactosidase S, β1-3 Galactosidase S, α1-2 Fucosidase, or α1-3,4 Fucosidase, or any combination thereof. In some embodiments, the installation can involve treating the plurality of enriched glycan-modified macromolecules with a monosaccharide-specific glycosidase, wherein the monosaccharide comprises sialic acid, mannose, N-Acetylglucosamine, or N-Acetylgalactosamine. In some embodiments, the glycosidase comprises α2-3,6,8 Neuraminidase, α1-2,3,6 Mannosidase, β-N-Acetylglucosaminidase S, or α-N-Acetylgalactosaminidase, or any combination thereof. In some cases, the installation can involve treating the plurality of enriched glycan-modified macromolecules with a glycosyltransferase to attach the glycan-linked isotopic label to a glycan of the enriched glycan-modified macromolecules. In some embodiments, the glycosyltransferase can be configured to perform an α2-6 sialylation, an α2-3 sialylation, a β1-4 galactosylation, a β1-3 galactosylation, an α1-2 fucosylation, an α1-3 fucosylation, a α1-4 fucosylation, a β1-3 N-acetylglucosaminylation, or a β1-6 N-acetylglucosaminylation, or any combination thereof. In some embodiments, the glycosyltransferase comprises α2-6 sialyltransferase 1 (ST6Gal1), α2-3 sialyltransferase 3/4 (ST3Gal3, ST3Gal4), β1-4 Galactosyltransferase 1 (β4GalT1), β1-3 Galactosyltransferase 5 (β3GalT5), Fucosyltransferase 1 or 2 (FucT1 or FucT2), Fucosyltransferase transferases 4 or 7 (FucT4 or FucT7), Fucosyltransferase 3 (FucT3), β1-3N-acetylglucosaminyltransferase 2 (B3GNT2), or β1-6 N-acetylglucosaminylation (GCNT2), or any combination thereof. In some embodiments, the glycan-linked isotopic label corresponds to a heavy isotope labeled derivative of the predetermined monosaccharide cleaved by a glycosidase. In some cases, the glycan-linked isotopic label comprises sialic acid or a derivative thereof, galactose or a derivative thereof, fucose or a derivative thereof, N-acetylglucosamine or a derivative thereof, N-acetylgalactosamine or a derivative thereof, mannose or a derivative thereof, glucose or a derivative thereof, or any combination thereof. In some embodiments, wherein the isotopic label comprises sialic acid or a derivative thereof, wherein the sialic acid or a derivative thereof comprises N-Acetylneuraminic acid (Neu5Ac), N-Glycolylneuraminic acid (Neu5Gc), or 2-keto-3-deoxy-D-glycero-D-galacto-nononic acid (KDN). In some embodiments, the glycan-linked isotopic label comprises a naturally-occurring monosaccharide comprising one, two, three, four, five or six ¹³C or ¹⁶O atoms.

The second (e.g., glycan-linked, optionally isotopic) label may provide for a mass offset to distinguish the double-labeled peptide standard from the endogenous proteins (e.g., the endogenous samples). The isotope label may provide for a mass offset to distinguish the double-labeled peptide standard from endogenous peptides on a mass spectrometer. The isotope label may provide for glycan specific triggering. The glycan specific triggering may comprise increasing the mass difference between two glycopeptides. The glycan specific triggering may comprise increasing the mass difference between the two glycopeptides to differentiate between the two glycopeptides on a mass spectrometer. The isotope label may increase the mass difference between the two glycopeptides by more than 0.1 Dalton (Da), more than 0.2 Da, more than 0.3 Da, more than 0.4 Da, more than 0.5 Da, more than 0.6 Da, more than 0.7 Da, more than 0.8 Da, more than 0.9 Da, more than 1 Da, more than 1.1 Da, more than 1.2 Da, more than 1.3 Da, more than 1.4 Da, or more than 1.5 Da. The isotope label may increase the mass difference between the two glycopeptides by less than 1.5 Da, less than 1.4 Da, less than 1.3 Da, less than 1.2 Da, less than 1.1 Da, less than 1.0 Da, less than 0.9 Da, less than 0.8 Da, less than 0.7 Da, less than 0.6 Da, less than 0.5 Da, less than 0.4 Da, less than 0.3 Da, less than 0.2 Da, or less than 0.1 Da.

In some aspects, the present disclosure provides for a set of mass spectrometry standards of a plurality of glycan-modified macromolecules, wherein a glycan-modified macromolecule of the plurality comprises a macromolecular backbone and at least one glycan modified macromolecular residue, comprising: a plurality of glycan-modified fragments of the glycan-modified macromolecules, wherein the plurality of glycan-modified fragments comprise: (a) a macromolecular backbone-linked isobaric label; and (b) a glycan-linked isotopic label. In some embodiments, the glycan-modified macromolecules comprise glycoproteins. In some embodiments, the plurality of glycan-modified fragments comprise fragments generated by treatment with an endoproteinase. In some embodiments, the endoproteinase comprises Trypsin, rLys-C, Lys-C, rAsp-N, chymotrypsin, Glu-C, or any combination thereof. In some embodiments, the plurality of glycan-modified fragments comprises: (i) peptides with C-terminal arginine or lysine; (ii) peptides with C-terminal lysine; (iii) peptides with C-terminal arginine; (iv) peptides with C-terminal tyrosine, phenylalanine, or tryptophan; or (v) peptides with C-terminal glutamate. In some embodiments, the macromolecular backbone-linked isotopic label comprises an isobaric labeling reagent. Examples of isobaric labeling methods include tandem mass labels (TMT), isobaric labels for relative and absolute quantification (iTRAQ), mass differential labels for absolute and relative quantification, and DiLeu labeling. Isobaric labels as described herein can include any of the isobaric labels described in e.g., FIG. 20A, 20B, 21, or 22, or any combination thereof. The isobaric label may comprise a mass reporter region, a linker region, a mass normalization region, a protein reactive group, or combinations thereof. The link region may be cleavable. The isobaric label may comprise amine-reactive labels. The isobaric label may react with cysteine residues. The isobaric label may react with carbonyl groups. The isobaric label may be used to help distinguish between the enriched or purified biological samples (e.g., the double-labeled peptide standard) from the endogenous samples. The isobaric labels may be used to distinguish the enriched or purified biological samples (e.g., the double-labeled peptide standard) from the endogenous samples on a mass spectrometer. The isobaric label may provide for multiplexing. In some embodiments, the isobaric labeling reagent comprises: (i) an amine-, thiol-, or carbonyl-reactive moiety and (ii) an ionizable moiety comprising a stable heavy isotope. In some embodiments, the glycan-linked isotopic label is specific for: (i) a disaccharide linkage type of the enriched glycan-modified macromolecules, or (ii) a monosaccharide of the glycan of the enriched glycan-modified macromolecules. In some embodiments, the glycan-linked isotopic label comprises sialic acid or a derivative thereof, galactose or a derivative thereof, fucose or a derivative thereof, N-acetylglucosamine or a derivative thereof, N-acetylgalactosamine or a derivative thereof, mannose or a derivative thereof, glucose or a derivative thereof, or any combination thereof. In some embodiments, the isotopic label comprises sialic acid or a derivative thereof, wherein the sialic acid or a derivative thereof comprises N-Acetylneuraminic acid (Neu5Ac), N-Glycolylneuraminic acid (Neu5Gc), or 2-keto-3-deoxy-D-glycero-D-galacto-nononic acid (KDN). In some embodiments, the glycan-linked isotopic label comprises a naturally occurring monosaccharide comprising one, two, three, four, five or six 13C or 16O atoms.

### Generation of a Double-Labeled Glycolipid Standard

Disclosed herein are methods for generating a double-labeled lipid standard. The double-labeled lipid standard may comprise a lipid. The double-labeled lipid standard may comprise a glycan. The double-labeled lipid standard may comprise a lipid and a glycan. The double-labeled lipid standard may comprise enriched or purified lipids comprising the glycan. The double-labeled lipid standard may be generated from a portion of the biological sample comprising the enriched or purified lipid. The double-labeled lipid standard may be generated from a portion of the biological sample that is a majority of the biological sample. The double-labeled lipid standard may be generated from a portion of the biological sample that is a 90% or more of the biological sample. The double-labeled lipid standard may be generated from a portion of the biological sample that is half or less than half of the biological sample. The double-labeled lipid standard may be generated from a portion of the biological sample that is half or more than half of the biological sample.

Generation of the double-labeled lipid standard may comprise adding a first mass label (aka tag). The first label may be an isotope label. The isotope label may be added to a lipid backbone portion of the enriched or purified portion of the biological sample. In some embodiments, the isotope label may be added to a lipid backbone portion of the enriched or purified portion of the biological sample.

Generation of the double-labeled lipid standard may comprise a second (e.g., glycan-linked) mass label. Generation of the double-labeled lipid standard may comprise adding the second label to the enriched or purified portion of the biological sample. The second label may be an isotope label. The isotope label may be added to the glycan portion of the enriched or purified portion of the biological sample. In some embodiments, the isotope label may be added to the lipid portion of the enriched or purified portion of the biological sample. In some embodiments, the isotope label may be added to the lipid portion, or the glycan portion, or a combination thereof. In some embodiments, the isotope label may be added to the lipid portion or the glycan portion through organic synthesis or enzymatic reactions, or combinations thereof. The isotope label may be added to the glycan portion of the enriched or purified portion of the biological sample by enzymes. The isotope label may be added to the glycan portion of the enriched or purified portion of the biological sample by one enzyme. The isotope label may be added to the glycan portion of the enriched or purified portion of the biological sample by more than one enzyme, more than two enzymes, more than three enzymes, more than four enzymes, more than five enzymes, more than six enzymes, more than seven enzymes, more than eight enzymes, more than nine enzymes, or more than ten enzymes. The isotope label may be added to the glycan portion of the enriched or purified portion of the biological sample by more than 10 enzymes, more than 20 enzymes, more than 30 enzymes, more than 40 enzymes, or more than 50 enzymes. The enzyme may trim a monosaccharide residue from a native glycan of the enriched or purified portion of the biological sample. The enzyme may trim more than one monosaccharide residues from the native glycan. The enzyme may add a labeled monosaccharide residue on the enriched or purified portion of the biological sample. The enzyme may add more than one labeled monosaccharide residues on the enriched or purified portion of the biological sample. The monosaccharide may comprise, but is not limited to, glucose, galactose, mannose, N-acetylglucoasmine, N-acetylgalactosamine, sialic acids, or combinations thereof. The sialic acids may comprise Neu5Ac, Neu5Gc, KDN, or combinations thereof. The monosaccharide residue from the native glycan and the labeled monosaccharide may be the same. The monosaccharide residue from the native glycan and the labeled monosaccharide residue may be different. The monosaccharide residue from the native glycan and the labeled monosaccharide residue may have the same mass. The monosaccharide residue from the native glycan and the labeled monosaccharide residue may have different masses.

The glycan-linked isotopic label can be specific for: (i) a disaccharide linkage type of the enriched glycan-modified macromolecules, or (ii) a monosaccharide of the glycan of the enriched glycan-modified macromolecules. The glycan-linked isotopic label can comprise a naturally-occurring monosaccharide comprising at least one, two, three, four, five, six, or more heavy isotope atoms.

Installing the second (e.g., glycan-linked, optionally isotopic) label may involve one or more enzymatic reaction using e.g., at least one enzyme described in Table 1 or Table 2. In some cases, the installation can involve treating a plurality of enriched glycan-modified macromolecules with a monosaccharide-specific or linkage-specific glycosidase to remove a predetermined monosaccharide from a glycan of the plurality of enriched glycan-modified macromolecules. In some embodiments, the installation can comprise treating the plurality of enriched glycan-modified macromolecules with a linkage-specific glycosidase, wherein the linkage-specific glycosidase comprises a glycosidase specific for an α2-3 sialic acid linkage, a β1-4 galactose linkage, a β1-3 galactose linkage, an α1-2 fucose linkage, or an α1-3/4 fucose linkage. In some embodiments, the linkage-specific glycosidase comprises α2-3 Neuraminidase S, β1-4 Galactosidase S, β1-3 Galactosidase S, α1-2 Fucosidase, or α1-3,4 Fucosidase, or any combination thereof. In some embodiments, the installation can involve treating the plurality of enriched glycan-modified macromolecules with a monosaccharide-specific glycosidase, wherein the monosaccharide comprises sialic acid, mannose, N-Acetylglucosamine, or N-Acetylgalactosamine. In some embodiments, the glycosidase comprises α2-3,6,8 Neuraminidase, α1-2,3,6 Mannosidase, β-N-Acetylglucosaminidase S, or α-N-Acetylgalactosaminidase, or any combination thereof. In some cases, the installation can involve treating the plurality of enriched glycan-modified macromolecules with a glycosyltransferase to attach the glycan-linked isotopic label to a glycan of the enriched glycan-modified macromolecules. In some embodiments, the glycosyltransferase can be configured to perform an α2-6 sialylation, an α2-3 sialylation, a β1-4 galactosylation, a β1-3 galactosylation, an α1-2 fucosylation, an α1-3 fucosylation, a α1-4 fucosylation, a β1-3 N-acetylglucosaminylation, or a β1-6 N-acetylglucosaminylation, or any combination thereof. In some embodiments, the glycosyltransferase comprises α2-6 sialyltransferase 1 (ST6Gal1), α2-3 sialyltransferase 3/4 (ST3Gal3, ST3Gal4), β1-4 Galactosyltransferase 1 (β4GalT1), β1-3 Galactosyltransferase 5 (β3GalT5), Fucosyltransferase 1 or 2 (FucT1 or FucT2), Fucosyltransferase transferases 4 or 7 (FucT4 or FucT7), Fucosyltransferase 3 (FucT3), β1-3N-acetylglucosaminyltransferase 2 (B3GNT2), or β1-6 N-acetylglucosaminylation (GCNT2), or any combination thereof. In some embodiments, the glycan-linked isotopic label corresponds to a heavy isotope labeled derivative of the predetermined monosaccharide cleaved by a glycosidase. In some cases, the glycan-linked isotopic label comprises sialic acid or a derivative thereof, galactose or a derivative thereof, fucose or a derivative thereof, N-acetylglucosamine or a derivative thereof, N-acetylgalactosamine or a derivative thereof, mannose or a derivative thereof, glucose or a derivative thereof, or any combination thereof. In some embodiments, wherein the isotopic label comprises sialic acid or a derivative thereof, wherein the sialic acid or a derivative thereof comprises N-Acetylneuraminic acid (Neu5Ac), N-Glycolylneuraminic acid (Neu5Gc), or 2-keto-3-deoxy-D-glycero-D-galacto-nononic acid (KDN). In some embodiments, the glycan-linked isotopic label comprises a naturally occurring monosaccharide comprising one, two, three, four, five or six ¹³C or ¹⁶O atoms.

In some cases, the second (e.g., glycan-linked) label can be added to a glycan chain of a glycopeptide or glycolipid described herein through the use of one or more enzyme described in Table 1 or Table 2.

The isotope label may provide a mass offset to distinguish the double-labeled lipid standard from the endogenous lipid samples (e.g., the non-enriched or non-purified portion of the biological sample). The isotope label may provide for a mass offset which distinguishes double-labeled lipid standard from the endogenous lipid samples on a mass spectrometer. The isotope label may provide for glycan specific triggering of MS2 spectrum acquisition. The glycan specific label may increase the mass difference between two glycolipids. The glycan specific label may increase the mass difference between the two glycolipids to differentiate between the two glycolipids on a mass spectrometer. The isotope label may increase the mass difference between the two glycolipids by more than 0.1 Dalton (Da), more than 0.2 Da, more than 0.3 Da, more than 0.4 Da, more than 0.5 Da, more than 0.6 Da, more than 0.7 Da, more than 0.8 Da, more than 0.9 Da, more than 1 Da, more than 1.1 Da, more than 1.2 Da, more than 1.3 Da, more than 1.4 Da, or more than 1.5 Da. The isotope label may increase the mass difference between the two glycolipids by less than 1.5 Da, less than 1.4 Da, less than 1.3 Da, less than 1.2 Da, less than 1.1 Da, less than 1.0 Da, less than 0.9 Da, less than 0.8 Da, less than 0.7 Da, less than 0.6 Da, less than 0.5 Da, less than 0.4 Da, less than 0.3 Da, less than 0.2 Da, or less than 0.1 Da.

### Isobaric Labeling of the Biological Sample

The endogenous proteins of the biological sample may be labeled. The endogenous proteins of the biological sample may comprise a portion of the biological sample that does not include the enriched or purified portion of the biological sample. The non-enriched or non-purified portion of the biological sample (e.g., the endogenous samples) may include the endogenous proteins of the biological sample. For example, FIG. 1 shows a 10% portion of the biological sample that is not enriched or purified and a 90% portion of the biological sample that is enriched or purified. The non-enriched or non-purified portion of the biological sample may be 10% or a minority of the biological sample. The non-enriched or non-purified portion of the biological sample (e.g., the endogenous samples) may be less than 1%, 2%, 3%, 4%, 5%, 10%, 20%, 30%, 40%, 50%, 60%, 70%, 80%, 90%, 95%, or less than 99% of the biological sample. The non-enriched or non-purified portion of the biological sample may be more than 99%, 95%, 90%, 80%, 70%, 60%, 50%, 40%, 30%, 20%, 10%, 5%, 4%, 3%, 2%, or more than 1% of the biological sample. The non-enriched or non-purified portion of the biological sample may be half of the biological sample, less than half, or more than half of the biological sample, or combinations thereof.

The non-enriched or non-purified portion of the biological sample (e.g., the endogenous samples) may be labeled. The label may be an isobaric label. The isobaric label may comprise a first isobaric tag. The isobaric tag may be added to the peptide portion of the non-enriched or non-purified portion of the biological sample. The isobaric tag may be a TMT reagent. Examples of additional isobaric labeling methods include isobaric tags for relative and absolute quantification (iTRAQ), mass differential tags for absolute and relative quantification, and DiLeu labeling. Isobaric tags as described herein can include any of the isobaric tags described in e.g., FIG. 20A, 20B, 21, or 22, or any combination thereof. The isobaric tags may comprise a mass reporter region, a linker region, a mass normalization region, a protein reactive group, or combinations thereof. The link region may be cleavable. The isobaric label may comprise amine-reactive tags. The isobaric label may react with cysteine residues. The isobaric label may react with carbonyl groups. The isobaric label may be used to help distinguish the non-enriched or non-purified portion of the biological samples (e.g., the endogenous samples) from the enriched or purified portion of the biological samples (e.g., the double-labeled peptide standard). The isobaric labels may be used to distinguish the enriched or purified portion of the biological sample from the non-enriched or non-purified portion of the biological sample (e.g., the endogenous samples) on a mass spectrometer. The isobaric label may provide for multiplexing.

In some aspects, the present disclosure provides for set of mass spectrometry standards of a plurality of glycan-modified macromolecules, wherein a glycan-modified macromolecule of the plurality comprises a macromolecular backbone and at least one glycan modified macromolecular residue, comprising: a plurality of glycan-modified fragments of the glycan-modified macromolecules, wherein the plurality of glycan-modified fragments comprise: a macromolecular backbone-linked isobaric tag; and a glycan-linked isotopic tag. In some cases, the glycan-modified macromolecules can comprise glycolipids.

### Identification/Measurement of the Endogenous Samples

Disclosed herein, in some aspects, are methods of identifying or measuring a physical parameter of an endogenous protein (or a fragment thereof) in a biological sample. The methods of identifying or measuring the physical parameter may include performing mass spectrometry to acquire a mass-to-charge ratio of at least one ionized species associated with an endogenous protein (or a fragment thereof) in a biological sample. The methods of identifying or measuring the physical parameter may include performing mass spectrometry on a single- or double-labeled peptide (e.g., glycopeptide) standard (e.g., labeled by any of the methods described herein) to acquire a mass-to-charge ratio of at least one ionized species associated with the single- or double-labeled peptide standard. The methods of identifying or measuring the physical parameter may include, based on a double-labeled peptide standard, identifying or measuring a physical parameter of an endogenous protein of a biological sample.

Mass spectrometry can be used for the determination of the elemental composition, mass to charge ratio, absolute abundance or relative abundance of an analyte (e.g., glycopeptides or glycolipids referred to herein). Mass spectrometric techniques can be useful for elucidating the composition (e.g., sequence of linked macromolecular residues in the case of proteins, glycans, or lipids or post-translational modifications on proteins) or abundance of analytes (e.g., abundance of glycopeptides, glycolipids, unmodified peptides, unmodified lipids, or PTM-modified peptides, or the presence of particular glycan or PTM modifications on peptides). Mass spectrometry can comprise ionizing an analyte (e.g., a proteolytically-digested glycopeptide or glycolipid) to generate charged species or species fragments, and a measuring mass-to-charge ratio or abundance of the analyte (e.g., precursor ions corresponding to the mass/charge ratio of a glycopeptide, peptide, or glycolipid, permitting evaluation of its presence or likely molecular composition, in a so-called MS1 analysis). Mass spectrometry can involve further fragmenting a charged species or species fragment to generate a product ion and measuring mass/charge ratio or abundance of the product ion (e.g., peptide, glycan, or PTM residue fragments, permitting elucidation of the residue components of precursor glycopeptides or glycolipids, or the presence of PTMs). Mass spectrometry data corresponding to analyte ion and analyte ion fragments can be provided as intensities of as a function of mass-to-charge (m/z) units (e.g., Thompson units) representing the mass-to-charge ratios of the analyte ions and/or analyte ion fragments.

In some cases, the mass spectrometry can be coupled with HPLC liquid chromatography for an LC MS analysis. Coupled chromatography-MS in such an LC MS analysis can improve performance of both chromatography on peptide, glycopeptide, and glycolipid species (e.g., proteolytically-digested peptides, PTM-modified peptides, or glycopeptides; or individual glycolipids) and mass spectrometric analysis of peptide, glycopeptide, and glycolipid species alone. A typical LC MS analysis can involve a first stage of a liquid chromatography column (e.g., an adsorption chromatography column, a partition chromatography column, an ion-exchange chromatography column, a size-exclusion chromatography column, or an affinity chromatography column) coupled to a second stage mass spectrometer that ionizes analytes as they are resolved on the liquid chromatography column and records mass-to-charge ratio of the resultant species. The first stage of LC-MS can report on the presence of individual peptide, PTM-peptide, glycopeptide, or glycolipid species (or fragments thereof) present in a sample via a chromatogram of total ion intensity or UV/Vis absorption peaks versus retention time, and a series of precursor (MS1) ions generated at each retention time. As individual chemical species in the sample will generally elute at continuous retention times, m/z spectra for each HPLC peak can provide a report of the individual starting constituents of a sample. The second stage of fragmentation MS (e.g., MS2) can be performed with selective triggering to further fragment precursor ions generating in the first stage mass spectrometry (MS1) to provide connectivity or modification information (e.g., identify glycan or PTM modifications of peptides or lipids via product ions corresponding to individual glycans, glycan fragments, PTMs, or PTM fragments).

The methods of identifying or measuring may include comparing a mass spectrum (e.g., individual m/z peaks) of an endogenous peptide to a mass spectrum of a single- or double-labeled peptide standard. The comparison may include using a double-labeled peptide standard to identify where mass-to-charge ratio of an endogenous peptide (or product ions thereof) are located in the mass spectrum. The comparison may include determining the amount of endogenous protein or occupancy of a modification on a protein (e.g., via characteristic m/z peaks of corresponding precursor or product ions). The methods of identifying or measuring may include matching the spectrum (e.g., individual m/z/peaks) of endogenous peptide comprising a PTM to a mass spectrometry library (e.g., to identify an m/z ratio corresponding to a predetermined peptide sequence plus a PTM, or to identify a characteristic product ion of the PTM).

In cases where separate samples for comparison are modified with isobaric tags and multiplexed, precursor or product ions originating from each individual sample can be identified by the predictable mass offset (e.g., m/z offset) provided by individual isobaric tags. In cases where a prepared double-labeled glycopeptide standard is provided within such samples, precursor or product ion peaks can be identified and distinguished from the precursor or product ions generated by the individual samples by the predictable mass offset of the peptide backbone mass, isotopic, or isobaric tag of the double-labeled glycopeptides in the standard. The peaks being identified, intensities of precursor or product ions corresponding to particular analytes within the samples (e.g., peptides of individual sequence, or complex glycans having a particular linkage of monosaccharides) can be compared to determine absolute abundances of particular analytes within the samples. In cases where a prepared double-labeled glycopeptide standard is provided within a sample where an isotopic glycan label is provided on a predetermined monosaccharide of peptidoglycans in the sample, the predictable mass offset (e.g., m/z offset) provided by the isotopic glycan label can be used to identify individual glycopeptide m/z peaks corresponding to glycopeptides containing the predetermined monosaccharide on a precursor or product ion spectrum. The individual glycopeptide m/z peaks being identified, their intensities can be compared to determine absolute abundances of particular glycopeptides containing the predetermined monosaccharide within the sample.

Disclosed herein, in some aspects, are methods of identifying the presence, abundance, or composition of an endogenous lipid in a biological sample, or measuring a physical parameter of an endogenous lipid in a biological sample. The methods of identifying the presence, abundance, or composition may include performing mass spectrometry. The methods of identifying the presence, abundance, or composition may include performing mass spectrometry on a double-labeled lipid standard. The methods of identifying the presence, abundance, or composition may include, based on the double-labeled lipid standard, identifying the presence, abundance, or composition of the endogenous lipid of the biological sample.

The methods of identifying the presence, abundance, or composition may include comparing a mass spectrum of the endogenous lipid to a mass spectrum of the double-labeled lipid standard. The comparison may include using the double-labeled lipid standard to identify where the endogenous lipid m/z peaks are located in the mass spectrum. The comparison may include determining an abundance of an endogenous lipid. The methods of identifying the presence, abundance, or composition may include obtaining a mass-to-charge ratio or abundance of an endogenous lipid. Obtaining the measurement of the endogenous lipid may include measuring a mass spectrum of the endogenous lipid. The methods of identifying or measuring may include matching the spectrum of endogenous lipids to a mass spectrometry library.

### Multiplexing

In some aspects, the biological sample may be combined with additional biological samples. The biological sample may be combined with one or more additional biological samples. The biological sample may be combined with two or more, three or more, four or more, five or more, six or more, seven or more, eight or more, nine or more, or ten or more additional biological samples. The biological sample may be combined with one or less, two or less, three or less, four or less, five or less, six or less, seven or less, eight or less, nine or less, or ten or less additional biological samples.

The additional biological sample may comprise the endogenous protein. The additional biological sample may comprise additional isobaric tags. The additional biological sample may comprise the endogenous lipid.

In some aspects, the methods of identifying or measuring the endogenous protein may include performing a multiplex measurement of the endogenous protein in the biological sample combined with the additional samples. FIG. 1, for example, shows samples 1, 2, and N measured and identified at the same time. N denotes the number of additional samples that may be included in the multiplex. In some aspects, N is equal to 3 or more, 4 or more, 5 or more, 6 or more, 7 or more, 8 or more, 9 or more, 10 or more, or 11 or more additional biological samples. The additional biological samples (N) may comprise additional endogenous proteins and additional isobaric tags. In some aspects, the methods of identifying or measuring the endogenous lipid may include performing a multiplex measurement of the endogenous lipid in the biological sample combined with the additional samples.

In some aspects, the methods described herein may include a second isobaric tag that identifies the second biological sample in the multiplex measurement. The methods may include additional isobaric tags that identifies the additional biological samples in the multiplex measurement. For example, in FIG. 1, sample 1 comprises endogenous protein 1 and isobaric tag 1, sample 2 comprises endogenous protein 2 and isobaric tag 2, and sample N comprises endogenous protein N and isobaric tag N. Isobaric tags as described herein can include any of the isobaric tags described in e.g., FIG. 20A, 20B, 21, or 22, or any combination thereof.

### Particle-Mediated Enrichment

Described herein, in some aspects, are measurement methods comprising: contacting the biological sample to capture biomolecules. Examples of biomolecules that may be captured by particles include proteins, transcripts, genetic material, or metabolites. Other examples of biomolecules that may be captured by particles include endogenous proteins. The captured biomolecules may make up a biomolecule corona around the particle.

Particles may be made from various materials. Such materials may include metals, magnetic particles, polymers, or lipids. A particle may be made from a combination of materials. A particle may comprise layers of different materials. The different materials may have different properties. A particle may include a core comprising one material and be coated with another material. The core and the coating may have different properties.

Such materials may include metals, magnetic particles, polymers, or lipids. A particle may be made from a combination of materials. A particle may comprise layers of different materials. The different materials may have different properties. A particle may include a core comprising one material and be coated with another material. The core and the coating may have different properties.

A particle may include a metal. For example, a particle may include gold, silver, copper, nickel, cobalt, palladium, platinum, iridium, osmium, rhodium, ruthenium, rhenium, vanadium, chromium, manganese, niobium, molybdenum, tungsten, tantalum, iron, or cadmium, or a combination thereof.

A particle may be magnetic (e.g., ferromagnetic or ferrimagnetic). A particle comprising iron oxide may be magnetic. A particle may include a superparamagnetic iron oxide nanoparticle (SPION).

Particles of various sizes may be used. The particles may include nanoparticles. Nanoparticles may be from about 10 nm to about 1000 nm in diameter. For example, the nanoparticles can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. A nanoparticle may be less than 1000 nm in diameter. Some examples include diameters of about 50 nm, about 130 nm, about 150 nm, 400-600 nm, or 100-390 nm.

### Identification of a Disease State in a Subject

The methods described herein may comprise inputting an abundance or presence of an endogenous protein or an endogenous lipid (or a PTM associated with the endogenous protein or endogenous lipid) from a sample from a subject into a classifier to evaluate a biological state. The methods described herein may be used to identify a biological state in a subject. The subject may be a human. The subject may be male or female. The subject may be a vertebrate. The subject may be a mammal. The subject may have a disease state. The subject may have a non-disease state. The subject may be in a healthy state.

The sample may be obtained for purposes of identifying a disease state in the subject. The sample may be obtained for purposes unrelated to identifying a disease state in the subject. The sample may be suspected as having the disease state or as not having the disease state in the subject. The methods described here may be used to confirm or refute the suspected disease state in the subject.

The biological state may be a healthy state. The biological state may be a disease state. The disease state may comprise a cancer. The cancer may comprise pancreatic cancer. The cancer may comprise lung cancer. The cancer may comprise breast cancer. The cancer may comprise colon cancer. The cancer may comprise liver cancer. The cancer may comprise ovarian cancer. The cancer may comprise pancreatic cancer, lung cancer, breast cancer, colon cancer, liver cancer, or ovarian cancer, or combinations thereof.

The classifier may be a method carried out by a computer system. The method may involve using measurements of biological samples to output a classification or prediction of a biological state. The method may use a classifier. The classifier may take biological measurements as input. The classifier may output a biological state. The classifier may be comprised of multiple steps such as, but not limited to, feature selection, feature transformation, latent space mapping, feature vector composition, feature weighting, input weighting, input into a model, output from a model, analysis of informative features, incorporation of pretrained models, transfer learning, fine-tuning of pretrained models, knowledge distillation, post-processing of model output. The model output may be post-processed. The classifier may be an artificial neural network, a support vector machine, a linear model, a non-linear model, a parametric model, a non-parametric model, a Bayesian model, a gaussian process, a binary classifier, a multilabel classifier, a non-binary classifier, a deep neural network, an ensemble method, a tree-based model, or a combination thereof. The model may be trained using a dataset composed of biological samples.

The model performance may be assessed using metrics such as, but not limited to, receiver operating curve area under the curve (ROCAUC), sensitivity-specificity curve, sensitivity-specificity area under the curve, precision-recall curve, precision-recall area under the curve, precision, recall, sensitivity, specificity, accuracy, f-measure, f1-measure, f2 measure or some combination thereof.

The performance of the model may be determined using at least one output of the model. The performance of the model may be determined using some or all of the internal state of the model. The performance of the model may be greater than about 20%. The performance of the model may be greater than about 30%. The performance of the model may be greater than about 40%. The performance of the model may be greater than about 50%. The performance of the model may be greater than about 60%. The performance of the model may be greater than about 70%. The performance of the model may be greater than about 75%. The performance of the model may be greater than about 77%. The performance of the model may be greater than about 79%. The performance of the model may be greater than about 80%. The performance of the model may be greater than about 82%. The performance of the model may be greater than about 84%. The performance of the model may be greater than about 86%. The performance of the model may be greater than about 88%. The performance of the model may be greater than about 90%. The performance of the model may be greater than about 91%. The performance of the model may be greater than about 92%. The performance of the model may be greater than about 93%. The performance of the model may be greater than about 94%. The performance of the model may be greater than about 95%. The performance of the model may be greater than about 96%. The performance of the model may be greater than about 97%. The performance of the model may be greater than about 98%. The performance of the model may be greater than about 99%. The classifier may be configured in a way to improve computational efficiency measure by, but not limited to, computational complexity, memory use, storage capacity, computational time, power requirements, storage and use on a smart phone, storage and use on a personal computer, storage and use on a cloud based system, storage and use on a high performance computer system, storage and use from a flash drive.

The methods for evaluating a disease state described herein may involve methods described in PCT/US2023/63358, which is incorporated by reference herein in its entirety. In some aspects, such methods can comprise: obtaining a data set comprising glycoprotein information from biomolecule coronas that correspond to physiochemically distinct particles incubated with a biofluid sample from a subject suspected of having a disease state; and applying a classifier to the data set to identify the biofluid sample as indicative of a healthy state or the disease state. In some embodiments, such methods can comprise: (a) obtaining a data set comprising amounts of at least 10 glycoproteins or glycopeptides from biomolecule coronas that correspond to particles incubated with a biofluid sample from a subject; and (b) applying a classifier to the data set to identify the biofluid sample as indicative of cancer or as not indicative of cancer. Some embodiments include identifying the subject as having the cancer. Some embodiments include identifying administering a cancer treatment to the subject. In some embodiments, the cancer comprises lung cancer. In some embodiments, the lung cancer comprises non-small cell lung cancer (NSCLC). In some embodiments, the NSCLC comprises stage 1, stage 2, or stage 3 NSCLC. In some embodiments, the NSCLC comprises stage 4 NSCLC. In some embodiments, the data set comprises first measurements of a readout indicative of the presence, absence, or amount of the at least 10 distinct glycoproteins or glycopeptides of the biomolecule coronas. In some embodiments, or glycopeptides further comprises generating second measurements having a sensitivity or specificity of about 80% or greater of being indicative of the subject having or not having the cancer. In some embodiments, obtaining the data set comprises detecting the at least 10 glycoproteins or glycopeptides by mass spectrometry, chromatography, liquid chromatography, high-performance liquid chromatography, solid-phase chromatography, a lateral flow assay, an immunoassay, an enzyme-linked immunosorbent assay, a western blot, a dot blot, immunostaining, sequencing, or a combination thereof. In some embodiments, obtaining the data set comprises detecting the at least 10 glycoproteins or glycopeptides by the mass spectrometry. In some embodiments, the classifier comprises features to distinguish between early-stage NSCLC and late-stage NSCLC. In some embodiments, the classifier comprises a supervised data analysis, an unsupervised data analysis, a machine learning, a deep learning, a dimension reduction analysis, a clustering analysis, or a combination thereof. In some embodiments, the clustering analysis comprises a hierarchical cluster analysis, a principal component analysis, a partial least squares discriminant analysis, a random forest classification analysis, a support vector machine analysis, a k-nearest neighbors analysis, a naive bayes analysis, a K-means clustering analysis, a hidden Markov analysis, or a combination thereof. In some embodiments, the glycoproteins or glycopeptides comprise multiple glycosylated versions of a same protein or a same peptide, respectively. In some embodiments, the glycoproteins or glycopeptides comprise different proteins or different peptides, respectively.

In some aspects, such methods can comprise: (a) contacting a sample (e.g., biofluid sample) of a subject with particles to form biomolecule coronas comprising at least 10 distinct glycoproteins or glycopeptides adsorbed to the particles; and (b) obtaining first measurements of the at least 10 distinct glycoproteins or glycopeptides. In some embodiments, obtaining the first measurements comprises combining the glycoproteins or glycopeptides with labeled or unlabeled glycoproteins or glycopeptides, or with labeled or unlabeled non-glycosylated forms of the glycoproteins or glycopeptides. In some embodiments, the method further comprises identifying second measurements as indicative of the subject having or not having have cancer. Some aspects include identifying the subject as having the cancer. Some aspects include identifying administering a cancer treatment to the subject. In some embodiments, the cancer comprises lung cancer. In some embodiments, the lung cancer comprises non-small cell lung cancer (NSCLC). In some embodiments, the NSCLC comprises stage 1, stage 2, or stage 3 NSCLC. In some embodiments, the NSCLC comprises stage 4 NSCLC. In some embodiments, the second measurements have a sensitivity or specificity of about 80% or greater of being indicative of the subject having or not having the cancer. In some embodiments, (b) comprises obtaining the first measurements of the at least 10 distinct glycoproteins or glycopeptides by mass spectrometry, chromatography, liquid chromatography, high-performance liquid chromatography, solid-phase chromatography, a lateral flow assay, an immunoassay, an enzyme-linked immunosorbent assay, a western blot, a dot blot, immunostaining, or sequencing, or a combination thereof. In some embodiments, (b) comprises obtaining the first measurements of the at least 10 distinct glycoproteins or glycopeptides by the mass spectrometry. In some embodiments, obtaining measurements of the at least 10 distinct glycoproteins comprise measuring a readout indicative of the presence, absence, or amount of the at least 10 distinct glycoproteins of the biomolecule coronas.

In some aspects, such methods can comprise: (a) contacting a sample (e.g., biofluid sample) from a subject with particles to form a biomolecule corona comprising proteins or peptides and glycoproteins or glycopeptides adsorbed to the particles; and (b) enriching the glycoproteins or glycopeptides, or separating the glycoproteins or glycopeptides from the proteins or peptides. In some embodiments, separating the glycoproteins or glycopeptides from the proteins or peptides comprises using liquid chromatography to separate the glycoproteins or glycopeptides from the proteins or peptides. In some embodiments, the liquid chromatography comprises hydrophilic interaction liquid chromatography (HILIC), electrostatic repulsion liquid chromatography (ERLIC) enrichments, high performance liquid chromatography (HPLC), or a combination thereof. In some embodiments, the liquid chromatography comprises multidimensional liquid chromatography. In some embodiments, the multidimensional liquid chromatography comprises two-dimensional electrophoresis.

In some aspects, such methods can comprise: (a) contacting a sample (e.g., biofluid sample) from a subject with particles to form a biomolecule corona comprising glycoproteins adsorbed to the particles; and (b) combining the glycoproteins or glycopeptides with labeled glycoproteins or glycopeptides. In some embodiments, (a) is performed prior to (b). In some embodiments, (a) is performed subsequent to (b). In some embodiments, (a) is performed during (b). In some embodiments, at least one of the glycoproteins or glycopeptides and at least one of the labeled glycoproteins or glycopeptides are the same. In some embodiments, at least one of the glycoproteins or glycopeptides and at least one of the labeled glycoproteins or glycopeptides are different. In some embodiments, the labeled glycoproteins or glycopeptides comprise an isotopic label, a mass tag, a barcode, a fluorescent label, a post-translation modification, a biomolecule from a same species of the subject, or a biomolecule from a species different than a species of the subject. In some embodiments, at least one of the labeled glycoproteins or glycopeptides have a predetermined amount. In some embodiments, each of the labeled glycoproteins or glycopeptides each have one predetermined amount. In some embodiments, the method further comprises measuring a readout indicative of the presence, absence, or amount of: (1) the glycoproteins or glycopeptides, (2) the labeled glycoproteins or glycopeptides, (3) a combination thereof. In some embodiments, the method further comprises generating the readout indicative of the presence, absence or amount of the glycoproteins or glycopeptides by comparing thereof with the readout indicative of the presence, absence or amount of the labeled glycoproteins or glycopeptides. In some embodiments, the method further comprises normalizing the readout indicative of the presence, absence or amount of the glycoproteins or glycopeptides with the readout indicative of the presence, absence or amount of the labeled glycoproteins or glycopeptides. In some embodiments, the method further comprises generating a combined readout indicative of the presence, absence or amount of the glycoproteins or glycopeptides using the readouts indicative of the presence, absence or amount of the glycoproteins or glycopeptides and the labeled glycoproteins or glycopeptides. Some aspects include calculating a ratio of glycosylated glycoprotein or glycopeptide over a total amount of glycosylated and non-glycosylated glycoprotein or glycopeptide.

In some aspects, such methods can comprise contacting a sample from a subject with particles to form a biomolecule corona comprising glycoproteins or glycopeptides adsorbed to the particles; and releasing at least one glycan moiety from the glycoproteins or glycopeptides adsorbed to the particles. Some aspects include separating the at least one glycan moiety from the glycoproteins or glycopeptides. Some aspects include combining the at least one glycan moiety with a labeled glycan moiety. In some aspects, the at least one glycan moiety and the labeled glycan moiety are a same glycan moiety. In some aspects, the at least one glycan moiety and the labeled glycan moiety are different glycan moieties. Some aspects include measuring an amount of the at least one glycan moiety or the labeled glycan moiety. Some aspects include measuring an amount of the at least one glycan moiety or the labeled glycan moiety by mass spectroscopy. In some aspects, a step is conducted in the presence of heavy water comprising an isotope. In some aspects, the heavy water comprises deuterium or ¹⁸O. Some aspects include introducing the isotope to a glycosylation site of the glycoproteins or glycopeptides that is de-glycosylated subsequent to a release of the at least one glycan moiety from the glycoproteins or glycopeptides. Some aspects include measuring an amount of at least one de-glycosylated glycoprotein or glycopeptide labeled by the isotope and an amount of glycoproteins or glycopeptides that are not labeled. Some aspects include calculating a ratio of the amount of at least one de-glycosylated glycoprotein or glycopeptide labeled by the isotope and the amount of glycoproteins or glycopeptides that are not labeled. In some aspects, the ratio may comprise the amount of at least one de-glycosylated glycoprotein or glycopeptide labeled by the isotope divided by a total amount comprising the amount of at least one de-glycosylated glycoprotein or glycopeptide labeled by the isotope and the amount of glycoproteins or glycopeptides that are not labeled.

In some aspects, such methods can include particles. In some embodiments, the particles comprise at least 2 different particles. In some embodiments, the particles comprise at least 3, 4, 5 or more different particles. In some embodiments, the particles comprise physiochemically distinct particles. In some embodiments, the physiochemically distinct particles comprise lipid particles, metal particles, silica particles, or polymer particles. In some embodiments, the physiochemically distinct particles comprise carboxylate particles, poly acrylic acid particles, dextran particles, polystyrene particles, dimethylamine particles, amino particles, silica particles, or N-(3-trimethoxysilylpropyl)diethylenetriamine particles.

Disclosed herein, in some aspects, are methods involve evaluating a cancer. In some aspects, the method further comprises identifying the subject as having a disease state such as cancer, and administering a treatment such as a cancer treatment to the subject.

In some aspects, such methods can include use of a sample. In some embodiments, the sample comprises a biofluid sample. In some embodiments, the biofluid comprises a blood sample that does not have red blood cells. In some embodiments, the biofluid comprises plasma or serum. In some embodiments, the biofluid comprises a blood sample that is essentially cell-free. In some embodiments, the biofluid is essentially free of red blood cells.

### Computer-Implemented Methods

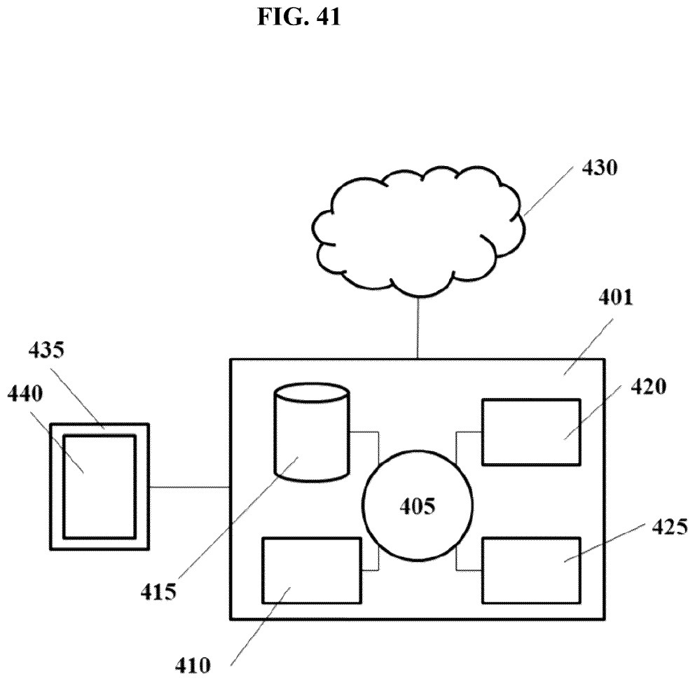

The present disclosure can provide for computer systems that are programmed to implement methods of the disclosure. FIG. 41 shows a computer system 401 that is programmed or otherwise configured to detect, compare, or analyze m/z (e.g., MS1, MS2) peaks or HPLC peaks from operations according to the disclosure (e.g., glycopeptide or glycolipid analyses via MS, LC/MS, or LC/MS/MS). The computer system 401 can regulate various aspects of the present disclosure, such as, for example, receive or determine m/z ratios of LC, MS1, or MS2 peaks, correlate sequences of peptides or structures of glycans to specific LC, MS1, or MS2 peaks, or utilize one or more LC, MS1, or MS2 peaks or detected ions associated with glycopeptides, glycolipids, or other PTM-modified molecules as described herein to detect a disease state. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple®iPhone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine-readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” generally in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, selecting LC, MS1, MS2, or m/z peaks for analysis in a mass spectrometry experiment, or interacting with graphical depictions of species detected by LC, MS1, MS2 and correlating the results among LC, MS1, MS2 analyses. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, calculate statistics measurements to identify antibodies and generate profiles or predict efficacy and toxicity of a treatment.

### EXAMPLES

The following examples are included for illustrative purposes only and are not intended to limit the scope of the invention.

### Example 1: Detecting Endogenous Glycopeptides Using Labeled Standards

A number (N) of blood, serum, or plasma samples are protease digested as shown in FIG. 1. Each sample is divided into a first portion (e.g., 90% of the total sample) and a second portion (e.g., 10% of the total sample).

The first portion of each sample is combined for glycopeptide enrichment or purification through hydrophilic interaction liquid chromatography (HILIC). The second 10% portion of each sample is subjected to an isobaric labeling procedure with corresponding reagents. Any suitable isobaric labeling scheme can be utilized, including tandem mass tags (TMT) or isobaric tag for relative and absolute quantitation (iTRAQ). In the instance where N is greater than 1, the samples can be labeled with serial derivatives of compatible isobaric labeling tags that have progressively higher molecular weights.

The enriched glycopeptide from the first portion of each sample is labeled with a first peptide-attached label. This first label is peptide backbone attached backbone label, and can be an isotopic label (e.g., an isobaric tag). The backbone provides a first reproducible mass offset common to all peptides originating in a given sample, allowing for multiple samples to be distinguished in a mass spectrometric trace. The enriched glycopeptides from the first portion of each sample are further modified with an isotope labeled monosaccharide (e.g., a glycan label). A portion of digest from each sample are pooled before first and second labeling. This serves to pool all samples so that all potential glycopeptides are in the pool. For example, about 10 to 20 μl from sample 1 can be combined with 10 to 20 μl from sample 2, 10 to 20 μl from sample 3, 10 to 20 μl from sample 4, and 10 to 20 μl from sample N to generate a pooled sample. Subsequently, first (backbone) and second (glycan) labeling is performed so that so that the double-labeled standards can be supplemented into the sample as standards and for multiplexing. The isotope labeled monosaccharide can comprise any of the structures shown in FIG. 6 and FIG. 7, including, but not limited to, sialic acid, galactose, fucose, N-acetylglucosamine, N-acetylgalactosamine, mannose, glucose, or combinations thereof. Sialic acids used may comprise specific sialic acid derivatives such as Neu5Ac, Neu5Gc, KDN, or combinations thereof. The incorporation of isotope labeled monosaccharide can involve the replacement of a native terminal monosaccharide, e.g., by trimming of a native terminal monosaccharide (e.g., sialic acid) by a monosaccharide-specific enzyme (e.g., a glycosidase) and the attachment of isotope labeled monosaccharide acid by a suitable glycosyltransferase. The isotope labeled monosaccharide can come from a commercial source or from an in vitro derivatization reaction. The backbone label and the isotopically labeled monosaccharide create a double-labeled glycopeptide. The double-labeled glycopeptide can be then used as a standard.

A first fraction of the double-labeled glycopeptides generated above is subjected to glycoproteomics analysis to generate a target list. The target list comprises mass spectrometry information of the double-labeled glycopeptide and endogenous proteins present in the samples.

A second fraction of the double-labeled glycopeptides is pooled with the second 10% portion of each sample. The pooled sample is subjected to targeted liquid chromatography mass spectrometry (e.g., LC-MS/MS) analysis. Endogenous proteins in each sample are identified and measured by matching the LC-MS data of the pooled sample to the library (e.g., by comparing the backbone- or glycan-labeled peaks to the peaks at expected non-labeled m/z ratios).

### Example 2. Isotopic Labeling of Glycans

To better identify the mass differentials of glycopeptides, glycan modifications on peptides are labeled with isotopic labels to permit distinction of glycan structural isomers (e.g., glycans having different branching or ester linkages but identical or closely related molecular formulas/molecular weights). As shown in FIG. 4, while mass labeling (e.g., isotopic or isobaric labeling) on the peptide may create a mass offset from endogenous peptides (see e.g., the bars corresponding to 3B and 3C in in the middle of the spectrum, which correspond to singly-labeled peptide) allowing species closely related in molecular weight to be distinguished, backbone labeling may still be insufficient for distinguishing glycan species similar in molecular weight. An illustrative example is shown in FIGS. 3A-3C, which illustrate examples of both monosaccharides and linked glycans that are difficult to resolve. FIG. 3A shows that a glycopeptide structure comprising one sialic acid (residual monoisotopic mass of 291.0954 Da) differs only by 1.0204 Da when compared to a glycopeptide structure comprising two fucose (residual monoisotopic mass of 292.1158 Da), indicating that a monosaccharide sialic acid modification is difficult to distinguish from a double fucose modification. FIG. 3B and FIG. 3C compare two common complex saccharide modifications (one di-sialylated biantennary complex type N-glycan and one biantennary complex type N-glycan with core-fucosylation and terminal sialyl Lewis A, respectively) which, while they comprise unique repertoires of monosaccharides, nonetheless are similar in monoisotopic mass (e.g., 2204.7724 vs 2205.7928), indicating that even complex saccharides of different composition can be difficult to distinguish. Glycan modifications can be provided on glycopeptides according to the disclosure by replacing monosaccharide constituents of complex glycans decorating glycopeptides with corresponding monosaccharides bearing a mass label (e.g., an isotopic label). For the purposes of illustration, the impact of example isotopic label modifications to the peptides of FIG. 3B and FIG. 3C are described in FIG. 4. Isotopically labeling two sialic acid residues (diamond-shaped residue) in the molecule of FIG. 3B alongside a TMT tag on the peptide backbone leads to a 2.75 Th (Thompson) offset from the endogenous glycopeptides at 4+ charge (compare 401 and 406 in FIG. 4). Labeling one sialic acid residue (diamond-shaped residue) alongside a TMT tag on the peptide backbone in FIG. 3C leads to a 2 Th offset from the endogenous glycopeptides at 4+ charge (compare 402 and 405 in FIG. 4). As shown in the graph depicted in FIG. 4, such glycan labeling (compare 405 and 406 in FIG. 4) can increase the mass difference between endogenous glycopeptides (compare 401 and 402 in FIG. 4) versus peptides with only a backbone label (see e.g., 403 and 404 in FIG. 4), further facilitating resolution of peptides of related mass/charge by separating them (405 and 406) from endogenous glycopeptides (401 and 406). Moreover, the glycan labeling is structure-specific that separates two glycopeptides of similar mass/charge further for better isolation (difference is 0.25 between 401 and 402 where it is 0.5 between 405 and 406).

Glycan modifications need not be limited to sialic acid or fucose. Additional examples of monosaccharide residues that can be isotopically labeled, as shown in FIG. 6 and FIG. 7, include, but are not limited to, galactose, N-acetylglucosamine, N-acetylgalactosamine, mannose, glucose, or combinations thereof. Sialic acids may further comprise Neu5Ac, Neu5Gc, KDN, or combinations thereof.

### Example 3. Measuring Endogenous Proteins with Synthetic Double-Labeled Glycopeptide Standards

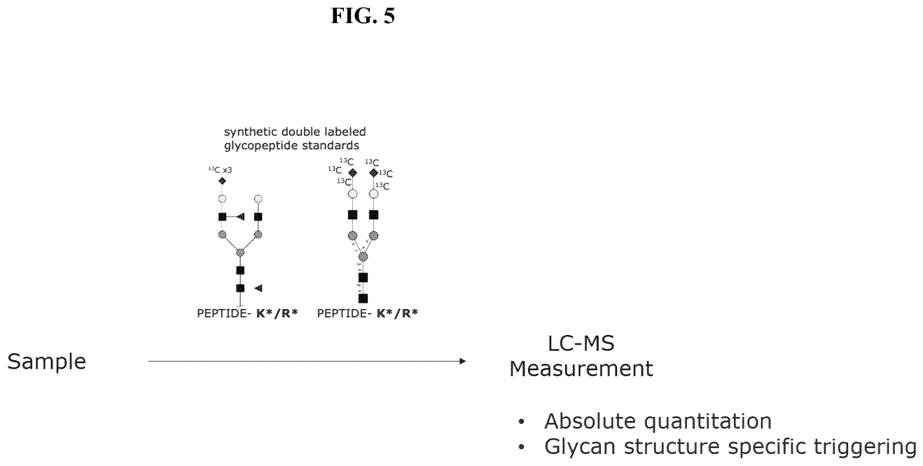

FIG. 5 shows an example workflow for analyzing a sample by supplementing synthetic double-labeled glycopeptide standards into the sample. In this workflow, the glycopeptide is labeled with an isotopic label on a peptide portion and an isotopic label on a glycan portion. The double isotope labeling on the peptide and the glycan portions may be synthesized by a commercial source. Such double-labeled peptides can be used to quantitate absolute amounts of peptides in the sample by detecting characteristic mass peaks due to the peptide standards and comparing those to putative equivalent mass peaks of unlabeled peptides in the sample.

### Example 4. Measuring Endogenous Lipids with Double-Labeled Glycolipid Standards

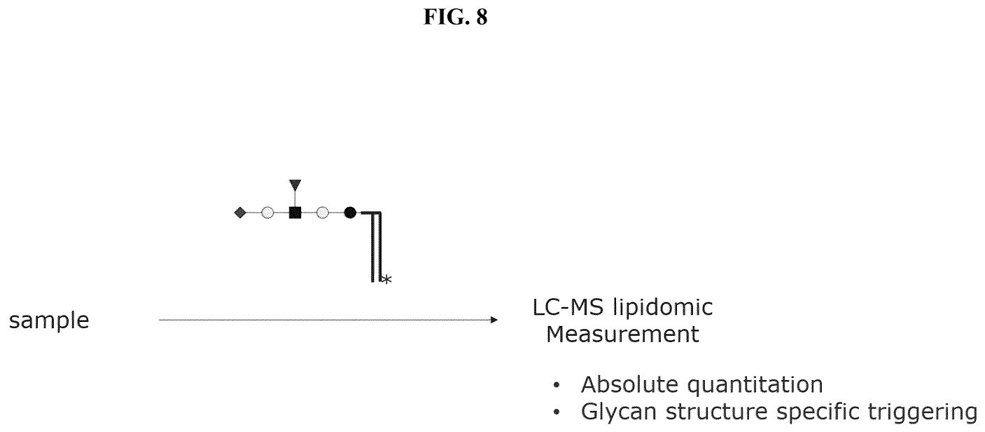

FIG. 8 shows an example workflow for analyzing a sample by supplementing double-labeled glycolipid standards into the sample. In this workflow, the glycolipid is labeled with an isotopic label on a glycan portion and an isotopic label on the lipid backbone. An example such a labeled glycolipid structure is shown in FIG. 7. The double isotope labeling on the glycan and the lipid portions may be synthesized by a commercial source, by standard organic synthesis techniques, or by in vitro enzymatic reactions. Such double-labeled lipids can be used to quantitate amounts of lipids in the sample by detecting characteristic mass peaks due to the lipid standards and comparing those to putative equivalent mass peaks of unlabeled lipids in the sample.

### Example 5. Combination of the Unique Functions from timsTOF and zenoTOF Enables In-Depth Glycoproteome Analysis

Glycosylation is an abundant and prominent post-translational modification (PTM) that may be found e.g., in a serum or plasma proteome, and can be associated with pathophysiological changes in protein activation, localization, trafficking, and turnover (e.g., in cancer pathophysiology). As a technique sensitive to mass-based changes associated with post-translational modification, mass spectrometry can be useful to study protein glycosylation associated with cancer or cancer-related pathophysiological changes. Previous work has shown the robustness of timsTOF and ZenoTOF for protein biomarker discovery; however, the potential of these instruments for mass spectrometry on protein glycosylation has not been fully explored. This study investigated how to combine unique features of timsTOF and ZenoTOF to enable in-depth glycoproteomic analysis and develop a new glycoprotein biomarker discovery platform. Objectives of this study were to develop glycoproteomics methods in timsTOF Pro 2 and ZenoTOF 7600, to combine the utilities of electron activated dissociation (EAD) from ZenoTOF and ion mobility from timsTOF to gain additional glycosylation-related, and to generate in-depth glycopeptide peptide fragment libraries to further biomarker discovery.

### Methods

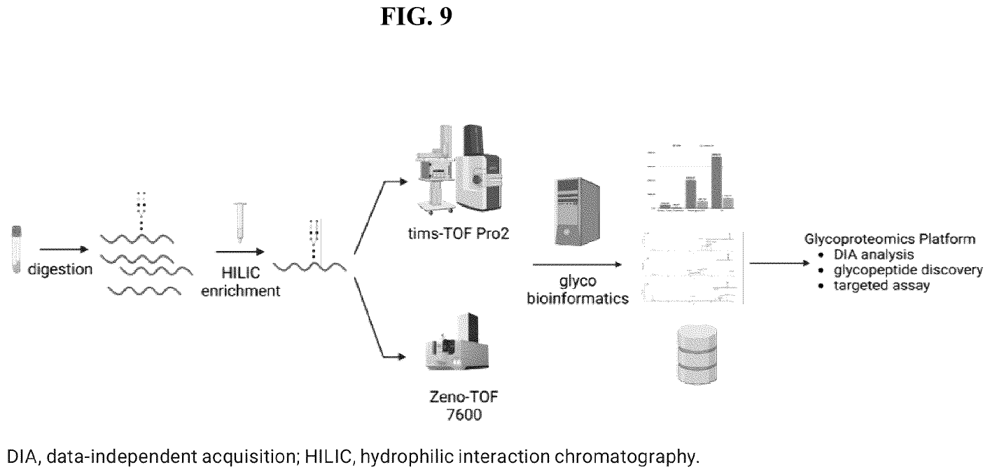

Pooled plasma (BioIVT) was digested by the iST sample digestion kit from PreOmics. Glycopeptides were enriched using HILICON iSPE hydrophilic interaction chromatography (HILIC) SPE cartridges. Ten micrograms (g) plasma equivalent of enriched glycopeptide was used for each injection. timsTOF Pro2 coupled with EvoSep and ZenoTOF 7600 coupled with a Waters M class LC system were used for data acquisition. Data were analyzed by Byonic using the 117 N-glycans database and a Byonic Score of 250 was used as the cutoff score. Further analysis was conducted using in-house written R scripts. The overall workflow that was used is shown in FIG. 9.

### Results

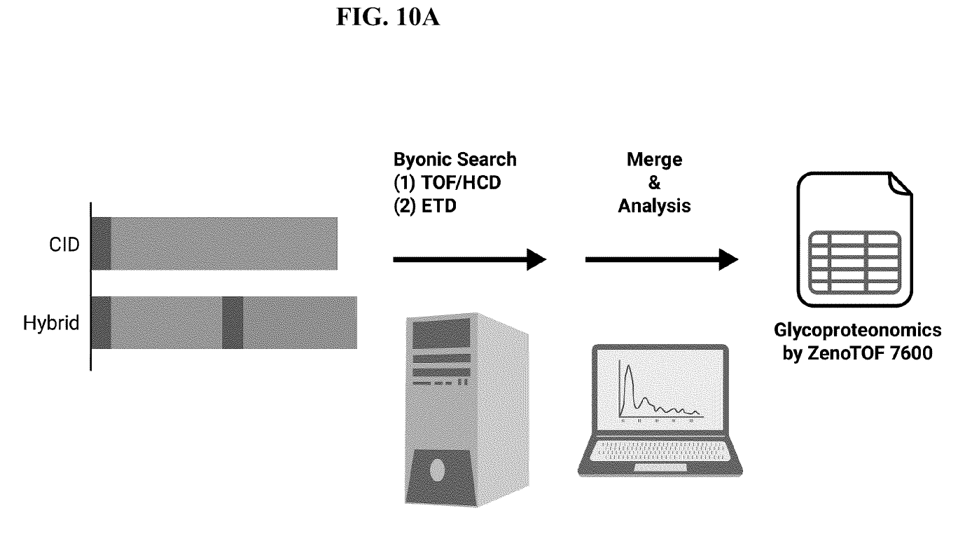

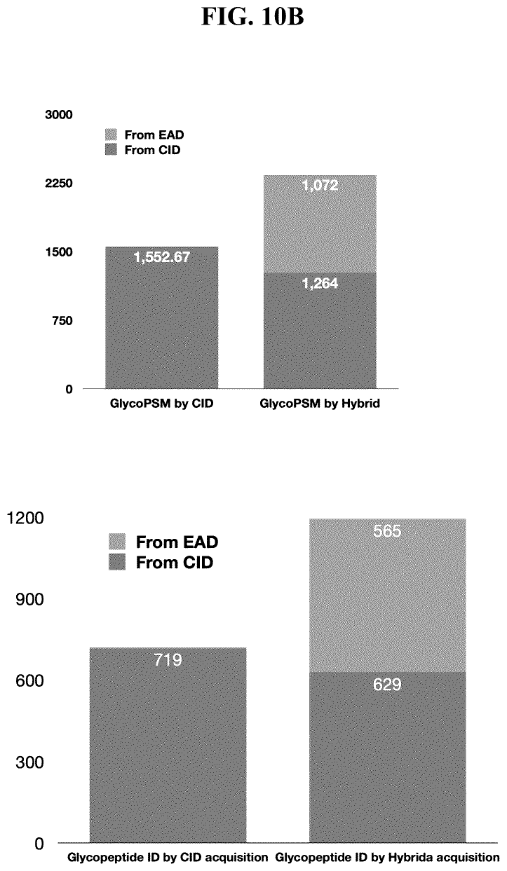

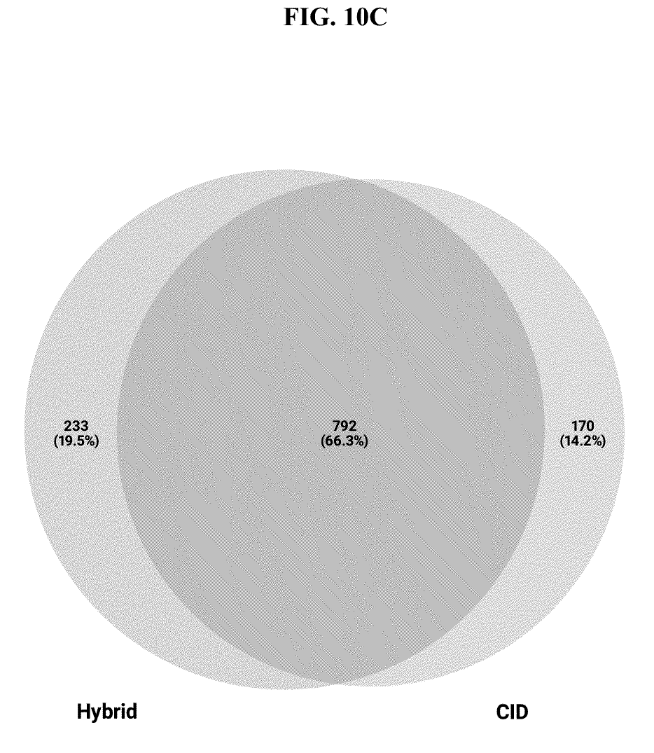

A liquid chromatography/mass spectrometry (LC/MS/MS) method comprised of both collision-induced dissociation (CID) and EAD MS2 acquisitions (hybrid) was designed and compared with CID only LC/MS acquisition (FIGS. 10A-10C). The resulting raw data were searched by Byonic first with the quadrupole time of flight/higher-energy collisional dissociation (TOF/HCD) parameter and subsequently with electron-transfer dissociation (ETD) parameters. The results were then merged and analyzed. In the hybrid mode, the average glycopeptide spectrum matches (PSMs) and IDs in each run were higher compared to the CID-only method. When condensed to unique glycopeptide IDs, the hybrid mode provided ˜20% of IDs that were not identified when using CID-only mode (FIG. 10C).

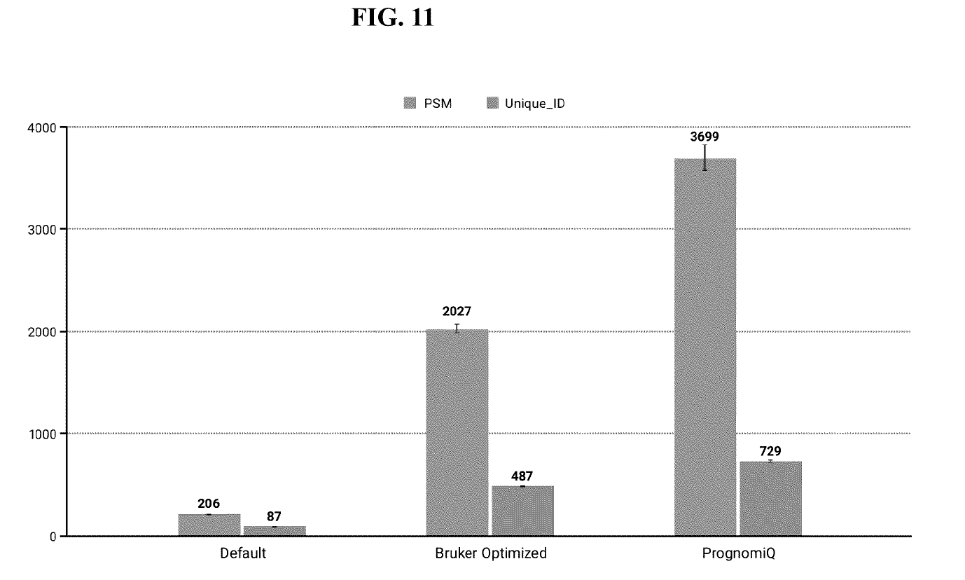

In developing a glycoproteomics method in timsTOF Pro2, stepped collision energy (SCE), along with other settings, were optimized to increase the number of unique glycopeptide IDs. Compared to the default method and the optimized method provided by Bruker, this method increases average peptide spectrum matched (PSMs) from 206 to 3699 and average unique glycopeptide IDs from 87 to 729 (FIG. 11).

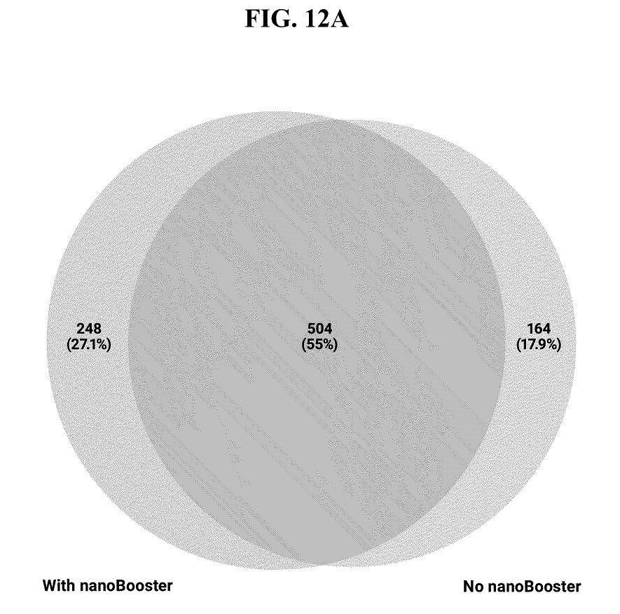

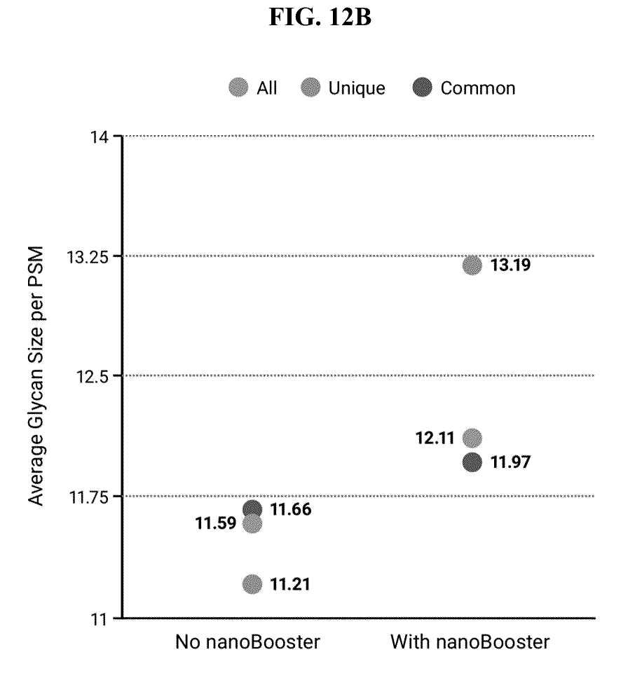

Glycopeptides identified with or without nanoBooster were analyzed. The nanoBooster provided an additional 27% of IDs compared to when no nanoBooster was used (FIG. 12A). Glycopeptides uniquely identified using nanoBooster carried larger glycans (FIG. 12B).

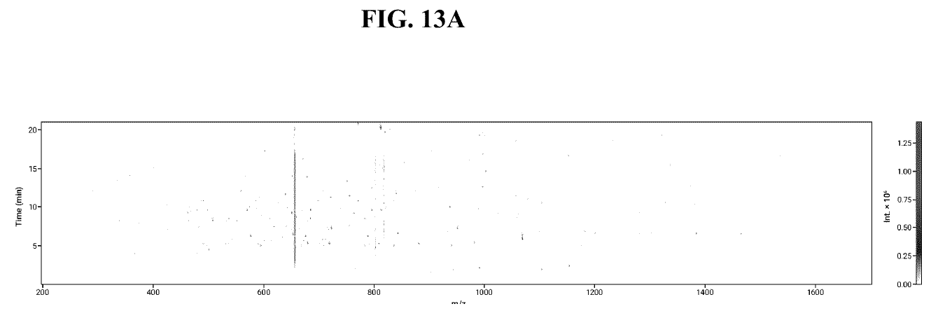

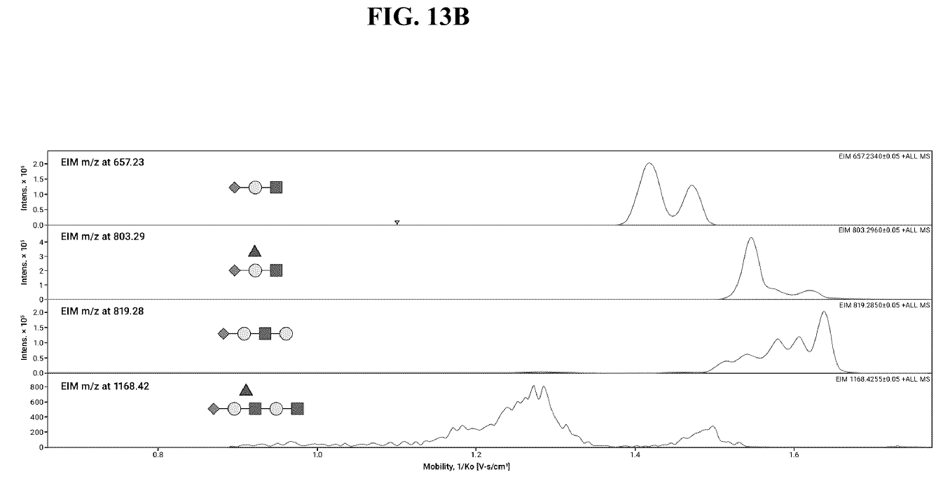

The heatmap of m/z over retention time showed ions presented across the whole retention time (FIG. 13A). These ions were found to be in-source fragmented glycan oxonium ions, and extracted ion mobilogram (EIM) showed separation of these oxonium ions (FIG. 13B). These results suggest that additional glycan structural information could be elucidated from the ion mobility data.

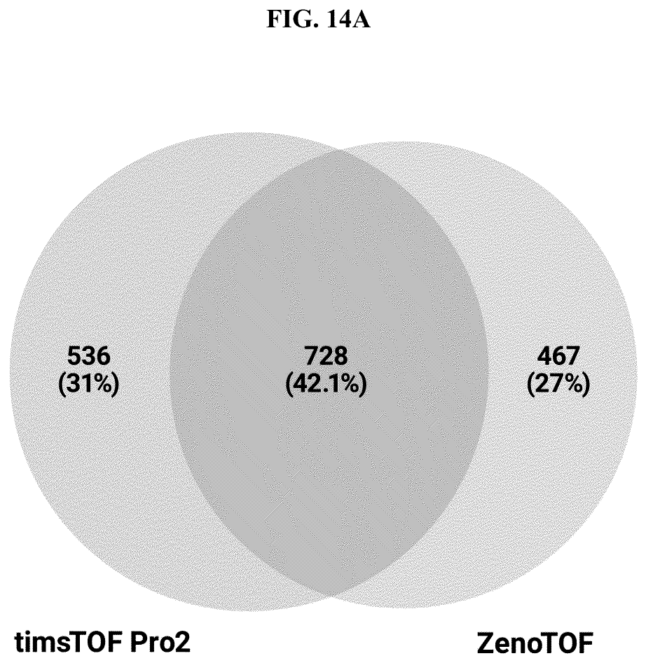

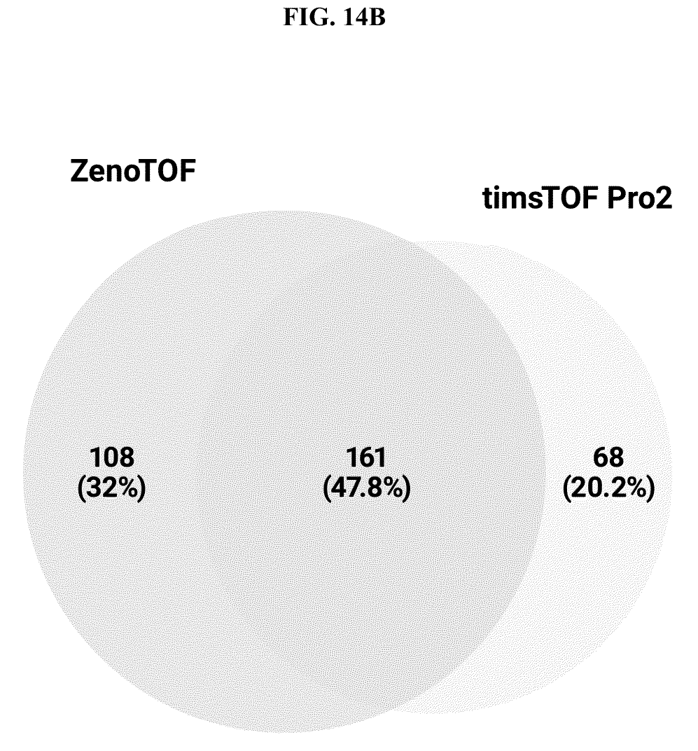

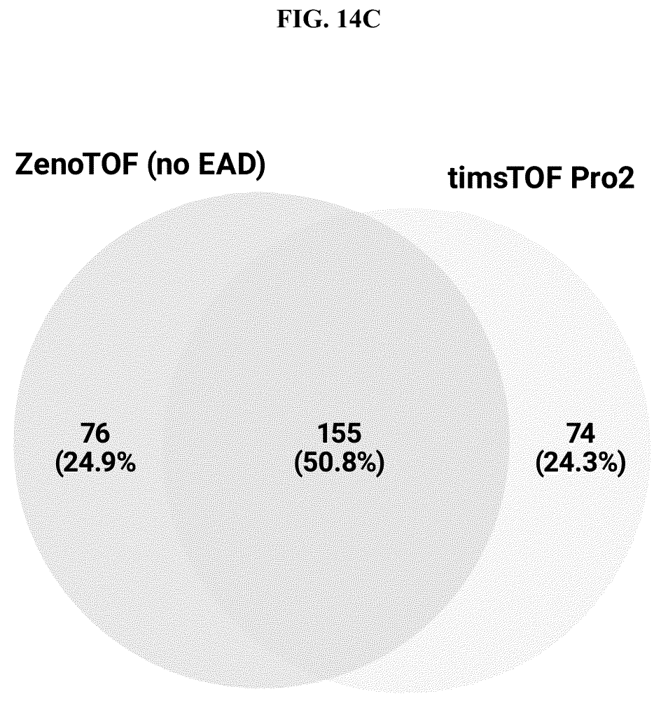

The results indicate tims-TOF and ZenoTOF categorize the glycoproteome differently. Only ˜42% of unique glycopeptides were identified by both the timsTOF Pro2 (with and without nanoBooster) and the ZenoTOF 7600 (CID and hybrid acquisition), and each instrument provided ˜30% unique glycopeptides (FIG. 14A). Results suggest that glycoproteome coverage can be enhanced through the unique capabilities of each instrument. When comparing stripped peptides, the overlap between ZenoTOF and timsTOF Pro2 increased to ˜48%, suggesting the two instruments detect glycan structures with different proclivities or sensitivities (FIG. 14B). When EAD (electron activated dissociation) data from ZenoTOF was excluded, the overlap increased further to 50.8%, suggesting some peptides were uniquely identified by the EAD feature in ZenoTOF (FIG. 14C).

### Conclusion

A hybrid LC/MS method comprised of both CID and EAD increased glycopeptide PSM and IDs. Combined CID and EAD (hybrid) increased glycopeptide IDs with a single acquisition in ZenoTOF 7600 system. SCE optimization was critical to glycopeptide identification. nanoBooster allowed for the identification of large glycopeptides in timsTOF Pro2. nanoBooster enabled identification of glycopeptides with larger glycans. timsTOF ion mobility elucidated additional structural information of glycopeptides. Optimization of the LC/MS method and the combination of unique features of the two platforms enhanced coverage of the glycoproteome. Glycoproteome knowledge may be useful for a multi-omics platform for biomarker discovery, and for disease screening methods.

### Example 6. Detailed Scheme for Labeling of Peptide Samples for Glycoproteomics

Two approaches were pursued to generate glycopeptide standards for glycoproteomics mass spectrometry studies: a synthetic peptide-based approach and a sample proteome derivatization-based approach.

### Synthetic Peptide-Based Approach (Procedure)

A peptide of VVLHPN(N4H5S2)YSQVDIGL*IK was synthesized by commercial provider where L* is an isotope labeled Leucine residue and N4H5S2 is a glycan structure attached to the peptide (N: N-acetylhexosamine, H: hexose; S: N-acetyl neuraminic acid or Neu5Ac). The two sialic acids (S) were removed by sialidase (New England BioLabs) to form VVLHPN(N4H5S0)YSQVDIGL*IK. Isotope labeled N-acetyl neuraminic acid (Cambridge Isotope Laboratories) was then reinstalled to form VVLHPN(N4H5S*2)YSQVDIGL*IK where the S* was an isotope labeled Neu5Ac. Linkage-specific sialidases/glycotransferases were then utilized to remove the native Neu5Ac and replace it with an isotope-labeled Neu5Ac.

The TMT labeled glycopeptides are then desialyated by treatment with sialidase at 37° C. for 1 hour, followed by heat inactivation at 95° C. for 5 minutes. After heat inactivation, sialic acid is then reinstalled by incubating the glycopeptides at pH 8.0 in the presence of MgCl2, CTP, isotope-labeled Neu5Ac, NmCSS and sialyltransferases (PmST for α2-3 linkage or PdST for α2-6 linkage) for 3 hours at room temperature.

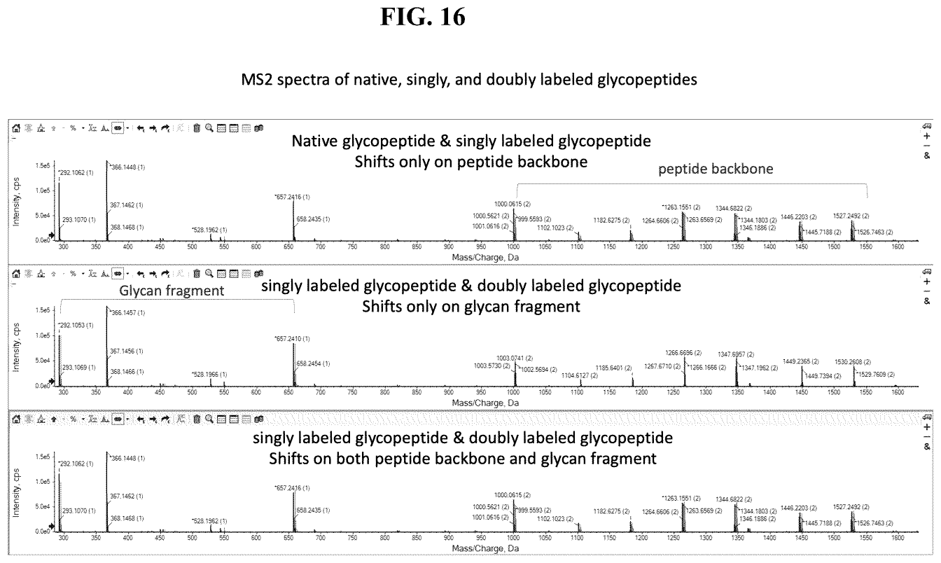

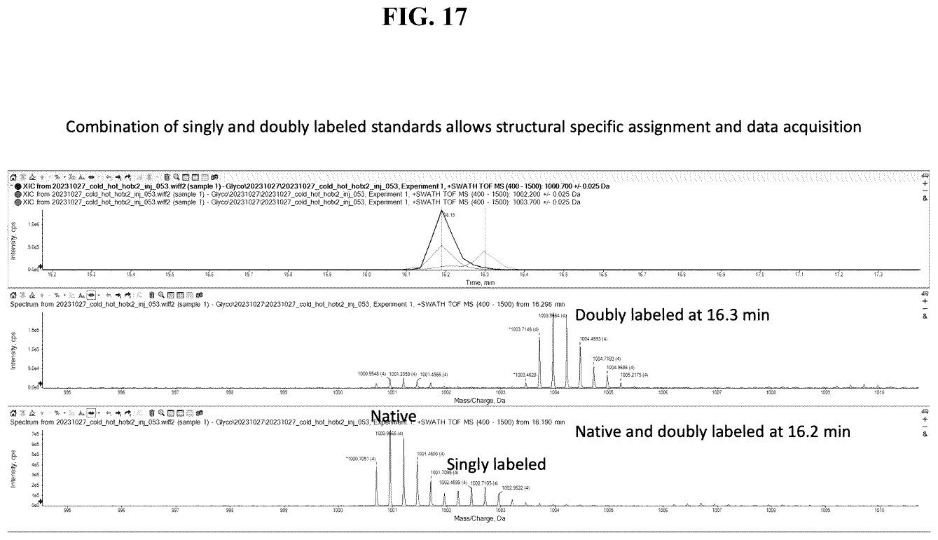

The resultant peptides were then analyzed by LC MS/MS, for which the MS1 spectra is shown in FIG. 15, the MS2 spectra is shown in FIG. 16, and the LC chromatography trace and associated MS2 spectra are shown in FIG. 17. The resultant spectra demonstrate that double labeling on the peptide backbone and glycan is useful for assignment for the peptide mass fragment peaks and the glycan fragment peaks, and that unlabeled, backbone-labeled, and double (backbone and glycan) labeled peptide can be clearly distinguished based on their MS1 and MS2 spectra (FIG. 15 and FIG. 16). The spectra further demonstrate that the α2-6 linkage glycan labeled peptides, but not α2-3 linkage elute at a similar retention time to the single- and un-labeled peptides (FIG. 17) demonstrating the double labeled standards could help structural assignment.

### Sample Proteome Derivatization-Based Approach (Procedure)

Starting glycopeptides are prepared by endoprotease digestion, in this case prepared from bovine fetuin by trypsin digestion. The digest is subjected to cleanup using C18 resin and is enriched for glycopeptides and divided into three fractions. Backbone labeling (TMT0—for the first fraction and TMT6—for the latter two fractions) was achieved with TMT labeling reagent followed by C18 cleanup. For the third TMT6-labeled fraction, Neu5Ac on the glycopeptides is then removed by sialidase and replaced with an isotope labeled Neu5Ac using an appropriate glycosyltransferase as described for the synthetic peptide-based approach.

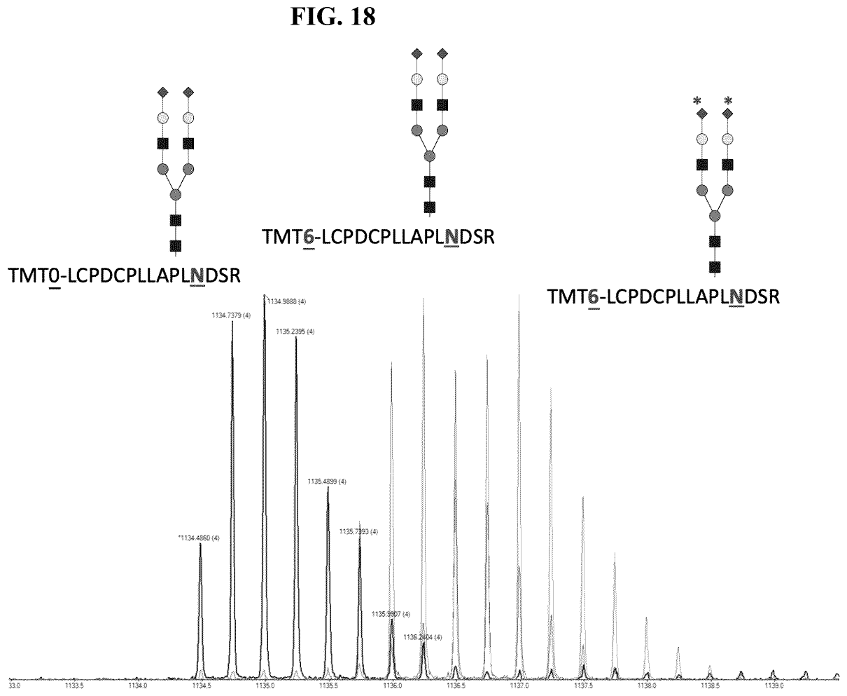

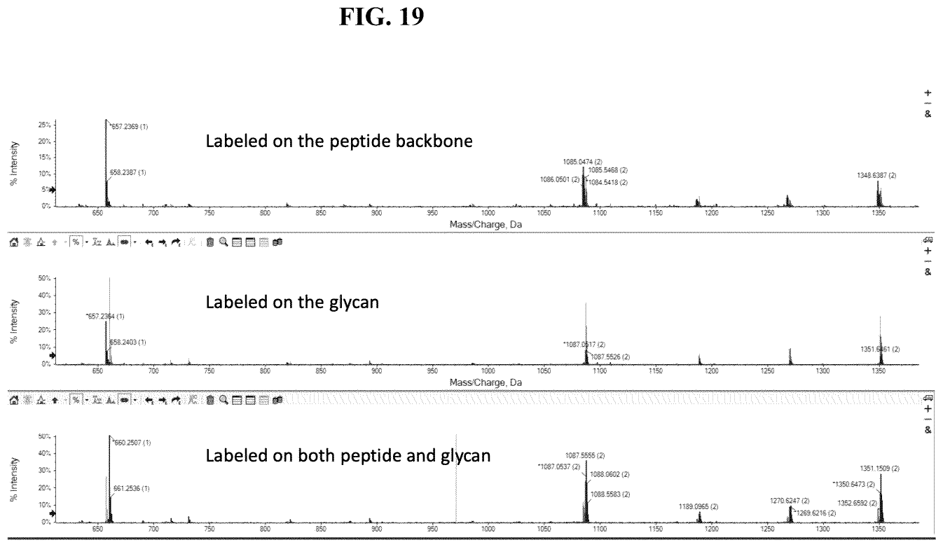

The resultant peptides were then analyzed by LC MS/MS, for which the MS1 spectra is shown in FIG. 18 and the MS2 spectra is shown in FIG. 19. The resultant spectra demonstrate that double labeling on the peptide backbone and glycan is useful for assignment for the peptide mass fragment peaks and the glycan fragment peaks, and that unlabeled, backbone-labeled, and double (backbone and glycan) labeled peptide can be clearly distinguished based on their MS1 and MS2 spectra (FIG. 18 and FIG. 19).

### Example 7: Enriching Glycopeptides from a Protease Digest

A protein mixture is isolated from a human plasma sample by means of a nanoparticle with a functionalized surface. The nanoparticles are rinsed and added to a buffer solution. The proteins are lysed from the nanoparticles and the particles are removed from the buffer. The proteins are digested using a protease. The device is prepared in a chromatography column using a C-18 bonded to silica as the reverse phase material for both the distal and proximal layers of reverse phase material. The HILIC layer is prepared using bare silica.

The device is conditioned by an organic phase of 80% acetonitrile containing 1% trifluoroacetic acid (80% ACN/1% TFA) and then equilibrated by an aqueous solvent of water and 1% TFA. The protein digest is acidified by TFA to a final concentration of 1% and loaded into the device. The device is washed with 1% TFA to wash away salt and other unbound small molecules. Peptides and glycopeptides are retained in the first layer of reverse phase material. Peptides and glycopeptides are eluted from first reverse layer by applying 80% ACN/1% TFA. Peptide are eluted out the device and glycopeptides are retained in the HILIC layer. The device is washed by 80% ACN/1% TFA to elute out peptides completely. Glycopeptides are eluted from HILIC layer by applying 1% TFA and glycopeptides are then retained in the second layer of reverse phase material. Glycopeptides are eluted out by 80% ACN/0.1% formic acid. The enriched glycopeptides are then analyzed using mass spectrometry to identify and quantify each glycopeptide fragment.

### Example 8. Glycopeptide Enrichment Experiments

### Glycopeptide Enrichment by Workflow 1 (FIG. 25).

A sample (e.g., pooled human female plasma) was protease digested as shown in FIG. 25. The digest (e.g., 100 μg) was dried and reconstituted in an organic mobile phase (e.g., 85% acetonitrile (ACN) with 1% trifluoroacetic acid (TFA)). Glycopeptide enrichment was conducted using an Agilent AssayMap Bravo system with HILIC stationary phase (e.g., CU cartridges). The cartridge was primed with a first volume (e.g., 100 μL with 1% TFA) and equilibrated with a second volume (e.g., 100 μL 85% ACN/1% TFA). The digest was loaded onto the HILIC stationary phase (e.g., CU cartridge) and washed with a third volume (e.g., 50 μL 85% ACN/1% TFA). The bound glycopeptides were eluted by a fourth volume (e.g., 70 μL 0.1% formic acid).

### Glycopeptide Enrichment by Workflow 2 (FIG. 26).

The same sample (e.g., pooled human female plasma digest) was used as in workflow 1. The digest (e.g., 100 μg) was cleaned by using an Agilent AssayMap Bravo system with a non-polar stationary phase (e.g., C18 cartridges). The non-polar stationary phase (e.g., C18 cartridges) were primed by using an organic mobile phase (e.g., 100 μL 85% ACN/1% TFA) and equilibrated with an aqueous mobile phase (e.g., 1% TFA). The digest was acidified by an additive (e.g., TFA) before loading to the non-polar stationary phase (e.g., C18 cartridges). The non-polar stationary phase (e.g., C18 cartridges) were washed using an aqueous mobile phase (e.g., 50 μL 1% TFA). The bound peptides were eluted with an organic mobile phase (e.g., 100 μL 85% ACN/1% TFA). The eluted peptides were immediately loaded onto a polar stationary phase (e.g., CU cartridges) that were primed with an aqueous mobile phase (e.g., 100 μL 1% TFA) and equilibrated with an organic mobile phase (e.g., 100 μL 85% ACN/1% TFA). The polar stationary phase (e.g., CU cartridges) were washed with an organic mobile phase (e.g., 50 μL 85% ACN/1% TFA). Bound glycopeptides are eluted from the polar stationary phase using an aqueous mobile phase (e.g., 70 μL 0.1% formic acid).

### One Device Enrichment (FIG. 27).

A polar stationary phase (e.g., CU cartridge) was stacked on a non-polar stationary phase (e.g., C18 cartridge) to create the one device enrichment device. The device was first primed using an organic mobile phase (e.g., 100 μL 85% ACN/1% TFA) and equilibrated using an aqueous mobile phase (e.g., twice of 50 μL 1% TFA). The sample (e.g., 100 μg pooled human female plasma digest) was acidified by an additive (e.g., TFA) and loaded on to the enrichment device. The devices were washed with aqueous mobile phase (e.g., twice with 50 μL 1% TFA). Peptides were eluted from the enrichment device using an organic mobile phase (e.g., 100 μL 85% ACN/1% TFA twice). Glycopeptides were eluted from the enrichment device using an aqueous mobile phase (e.g., 100 μL 2% ACN/0.1% formic acid).

For comparison, aliquots of the same sample (e.g., 100 μg human pooled female plasma digest) was dried and reconstituted in 85% ACN/1% TFA for HILIC enrichment (e.g., manually) using the polar stationary phase (e.g., CU cartridges). The polar stationary phase (e.g., CU cartridge) was primed using aqueous mobile phase (e.g., 50 μL 1% TFA) and equilibrated using organic mobile phase (e.g., 100 μL 85% ACN/1% TFA). The sample was loaded onto the polar stationary phase (e.g., CU cartridge). The polar stationary phase (e.g., CU cartridges) was washed using organic mobile phase (e.g., 100 μL 85% ACN/1% TFA) one or more times (e.g., twice). Glycopeptides were eluted from the polar stationary phase using aqueous mobile phase (e.g., 100 μL 2% ACN/0.1% formic acid).

### LC-MS Analysis.

For peptide proteomics, a sample (e.g., 200 ng) was injected for each LC-MS run in a Sciex ZenoTOF 7600 system. The data was analyzed by Spectronaut 17.1 with an in-house spectral library search. Glycopeptides were analyzed by a Sciex ZenoTOF 7600. The raw data was searched by Byonic for further analysis.

### Results of Workflow 1 vs Workflow 2 (FIG. 25 and FIG. 26).

Samples (e.g., eight) from the digest (without any clean up) and the peptide fraction following enrichment with both non-polar and polar stationary phases were analyzed by a proteomics approach. The Venn diagram (FIG. 29) shows an 86%, or 4688 constituents, overlapping in their precursor IDs suggesting the constituents between the peptide fraction of workflow 1 and the peptide fraction of workflow 2. Coefficient variation (CV) values are tight for both fractions suggesting high reproducibility within each fraction (FIG. 28). Tau score, a way to evaluate the correlation of component ranking between two data sets, is 0.93 for the two fractions (FIG. 30). This suggests the two fractions are similar in precursor identifications as well as their quantitation. Overall, the results suggest that the peptide fraction in workflow 2 could be used for the general proteomics purpose. Workflow 2 could be adopted to ProteoGraph to enable a deeper analysis.

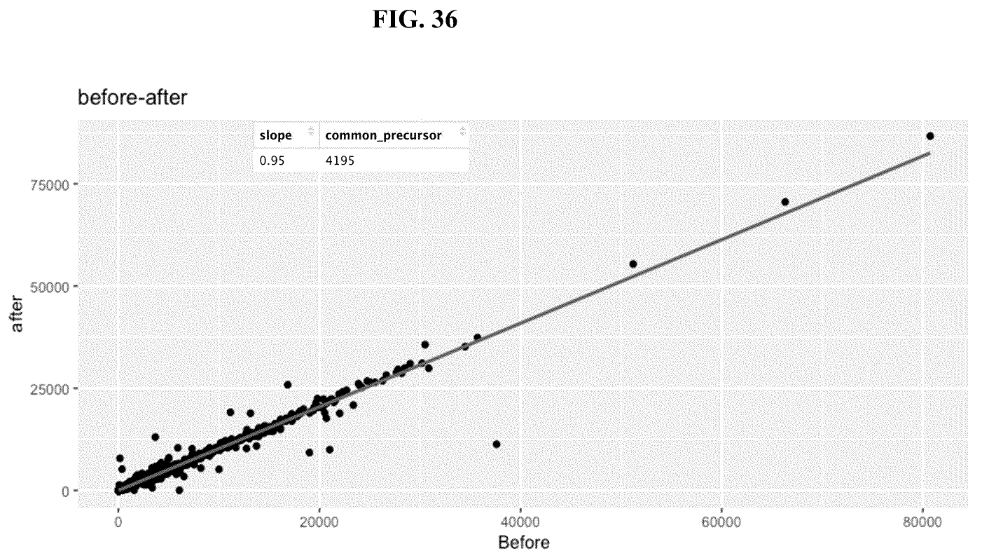

Glycopeptides following enrichment by workflow 1 and workflow 2 were subjected to glycoproteomics analysis. The glycoproteomics search using software specific for glycopeptide analysis (e.g., Byonic) showed the numbers of peptide spectrum match (PSM) and unique glycopeptide ID from workflow 2 are two times higher compared to the numbers of peptide spectrum match (PSM) and unique glycopeptide ID of workflow 1 (FIG. 36). The Venn diagram showed that workflow 2 covered 95.3% over the total identification of unique glycopeptide ID meaning the glycopeptides identified in workflow 1 were included in workflow 2 (FIG. 37). Further analysis of the MS2 spectra showed 92% of the MS2 spectra are glycopeptides in workflow 2 whereas the percentage of glycopeptides in workflow 1 is 69.7% (FIG. 38). The result shows the enrichment specificity is higher in workflow 2 compared to workflow 1.

### Example 9. Enrichment Device

Results from workflow 2 suggested the cleanup of digest and enrichment of glycopeptides could be combined into one workflow without reducing the performance. The two stationary phases (e.g., non-polar and polar) were merged into one device (FIG. 27). The proteomics analysis showed an 86.9% overlap in precursor identification between the digest input (before loading on to the device for enrichment) and the peptide fraction (following enrichment using the one device) are very similar (FIG. 35) and reproducible (FIG. 34). Nearly 87% overlapping in precursor identification, Tau score of 0.92, and the high linearity (e.g., slope of 0.95) of the quantitative ratio all indicate the peptide constituents are similar qualitatively and quantitatively between the two fractions (FIG. 36). Glycoproteomics showed the one device method has better glycopeptide enrichment efficiency compared to workflow 1 (FIGS. 27 to 29).

### Example 10. Metal Oxide Chromatography Combination

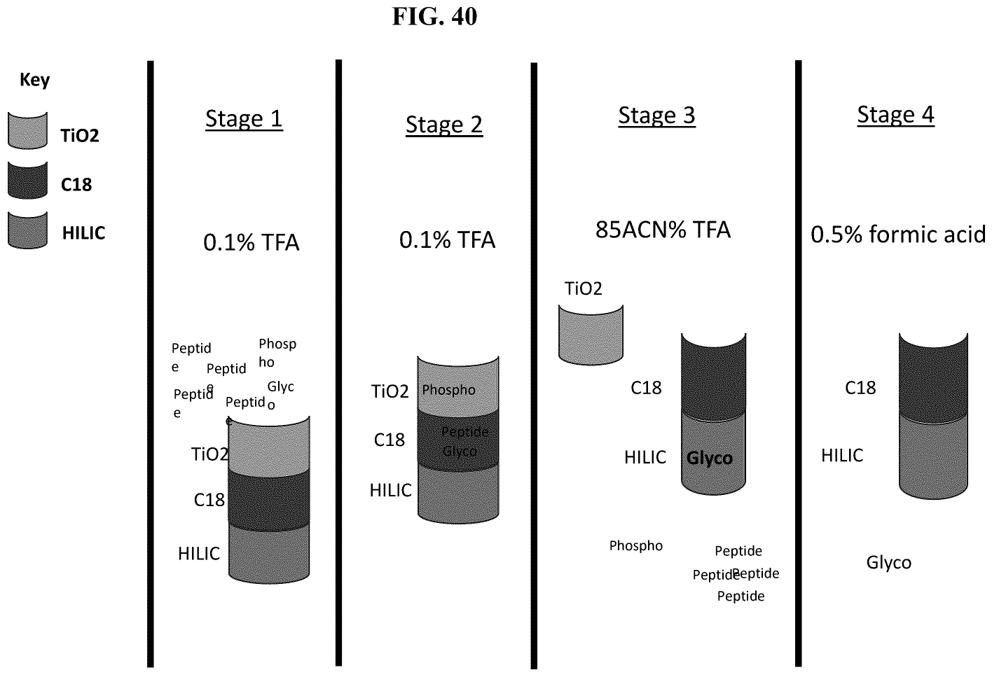

As shown in FIG. 40, a metal oxide layer (e.g., titanium dioxide) was placed on top of a non-polar stationary phase (e.g., C18 layer) and a polar stationary phase (e.g., HILIC layer). The three layers were pretreated (e.g., primed and/or equilibrated) with aqueous mobile phase (e.g., 0.1% TFA). After pretreating, the sample (e.g., biofluid) was added and washed with an aqueous mobile phase (e.g., 0.1% TFA), separating the peptides and glycopeptide products from the phosphopeptide products. The metal oxide layer (e.g., titanium dioxide) was removed from the C18 and HILIC layers. Then the non-polar stationary phase (e.g., C18 layer) and the polar stationary phase (e.g., HILIC layer) were washed with organic mobile phase (e.g., 85% ACN/1% TFA), removing the other peptide products (e.g., peptides, phosphopeptides) from the glycopeptides. Once the other peptide products (e.g., peptides, phosphopeptides) are removed, the non-polar stationary phase (e.g., C18 layer) and the polar stationary phase (e.g., HILIC layer) can be washed using an aqueous mobile phase (e.g., 0.5% formic acid) to elute the glycopeptides.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Embodiments A

The following embodiments are not intended to be limiting in any way.
1. A measurement method, comprising:
combining a biological sample with a double-labeled peptide standard comprising a peptide comprising a first label, and comprising a post-translational modification (PTM) comprising a second label; and
identifying or measuring, based on the double-labeled peptide standard, an endogenous protein of the biological sample, wherein the endogenous protein comprises the PTM.
2. The method of embodiment 1, wherein the PTM comprises a glycan, ubiquitin, phosphate, or acetyl.
3. The method of embodiment 1 or 2, wherein the endogenous protein comprises a glycoprotein.
4. The method of any one of embodiments 1-3, wherein the biological sample comprises a biofluid.
5. The method of embodiment 4, wherein the biofluid comprises blood, serum, plasma, urine, tears, semen, milk, vaginal fluid, mucus, saliva, or sweat, or combinations thereof.
6. The method of any one of embodiments 1-5, wherein the biological sample is obtained from a subject.
7. The method of embodiment 6, wherein the subject is a human.
8. The method of any one of embodiments 1-7, further comprising enriching or purifying the biological sample for peptides comprising the PTM prior to generating the double-labeled peptide standard.
9. The method of embodiment 8, wherein enriching or purifying the biological sample for peptides comprising the PTM comprises performing chromatography.
10. The method of embodiment 9, wherein the chromatography comprises hydrophilic interaction liquid chromatography (HILIC), liquid chromatography, solid-phase chromatography, column chromatography, affinity chromatography, ion exchange chromatography, or size exclusion chromatography, or combinations thereof.
11. The method of any one of embodiments 1-10, wherein the first label comprises an offset label.
12. The method of any one of embodiments 1-11, wherein the endogenous protein of the biological sample comprises a first isobaric tag.
13. The method of any one of embodiments 1-12, wherein the biological sample is combined with additional biological samples.
14. The method of embodiment 13, wherein the additional biological samples comprise additional isobaric tags and the endogenous protein.
15. The method of embodiment 11, wherein the offset label comprises a similar chemical structure as the first isobaric tag on the endogenous protein of the biological sample.
16. The method of embodiment 11, wherein the offset label comprises a higher molecular mass than the first isobaric tag on the endogenous protein of the biological sample.
17. The method of any one of embodiments 1-16, wherein identifying or measuring the endogenous protein comprises performing a multiplex measurement of the endogenous protein in the biological sample combined with the additional biological samples.
18. The method of any one of embodiments 12-17, wherein the first isobaric tag identifies the biological sample in the multiplex measurement, and wherein the additional isobaric tags identify the additional biological samples in the multiplex measurement.
19. The method of any one of embodiments 1-18, wherein the second label comprises an isotope label.
20. The method of any one of embodiments 1-19, wherein the second label is added to the double-labeled peptide standard by enzymes.
21. The method of any one of embodiments 1-20, further comprising generating the double-labeled peptide standard.
22. The method of embodiment 21, wherein generating the double-labeled peptide standard comprises adding the first label to a portion of the biological sample.
23. The method of embodiment 21 or 22, wherein generating the double-labeled peptide standard comprises adding the first label and the second label to the portion of the biological sample.
24. The method of any one of embodiments 21-23, wherein generating the double-labeled peptide standard comprises pooling the portion of the biological sample and the additional biological samples for enriching or purifying glycopeptides.
25. The method of any one of embodiments 1-24, wherein identifying or measuring, based on the double-labeled peptide standard, the endogenous protein of the biological sample comprises performing mass spectrometry.
26. The method of any one of embodiments 1-25, wherein identifying or measuring, based on the double-labeled peptide standard, the endogenous protein of the biological sample comprises comparing a mass spectrum of the endogenous protein to a mass spectrum of the double-labeled peptide standard.
27. The method of any one of embodiments 1-26, further comprising contacting the biological sample with a particle to capture the endogenous protein on the particle.
28. The method of embodiment 27, wherein the particle comprises a nanoparticle.
29. The method of any one of embodiments 1-28, further comprising adding the endogenous protein comprising the PTM to a mass spectrometry library.
30. The method of any one of embodiments 1-29, further comprising obtaining a measurement of the endogenous protein.
31. The method of any one of embodiments 1-30, wherein obtaining the measurement of the endogenous protein comprises measuring a mass spectrum of the endogenous protein.
32. The method of any one of embodiments 1-31, further comprising inputting the measurement of the endogenous protein into a classifier to evaluate a biological state.
33. The method of embodiment 32, wherein the biological state comprises a healthy state.
34. The method of embodiment 32 or 33, wherein the biological state comprises a disease.
35. The method of embodiment 34, wherein the disease comprises a cancer.
36. the method of embodiment 35, wherein the cancer comprises pancreatic cancer.
37. The method of embodiment 35 or 36, wherein the cancer comprises lung cancer, breast cancer, colon cancer, liver cancer, or ovarian cancer.
38. A method, comprising:
contacting a biological sample with a particle to adsorb an endogenous protein of the biological sample to the particle, wherein the endogenous protein comprises a post-translational modification (PTM); and
combining the adsorbed endogenous protein with a double-labeled peptide standard comprising an isotope labeled amino acid residue on the peptide and a labeled version of the PTM.
39. The method of embodiment 38, further comprising identifying or measuring the endogenous protein based on the double-labeled peptide standard.
40. A measurement method, comprising:
combining a biological sample with a double-labeled lipid standard comprising a lipid comprising a first label, and comprising a glycan comprising a second label; and
identifying or measuring, based on the double-labeled lipid standard, an endogenous lipid of the biological sample, wherein the endogenous lipid comprises the glycan.
41. The method of embodiment 40, wherein the endogenous lipid comprises a glycolipid.
42. The method of embodiment 40 or 41, wherein the biological sample comprises a biofluid.
43. The method of embodiment 42, wherein the biofluid comprises blood, serum, plasma, urine, tears, semen, milk, vaginal fluid, mucus, saliva, or sweat, or combinations thereof.
44. The method of any one of embodiments 40-43, wherein the biological sample is obtained from a subject.
45. The method of embodiment 44, wherein the subject is a human.
46. The method of any one of embodiments 40-45, further comprising enriching or purifying the biological sample for lipids comprising the glycan prior to generating the double-labeled lipid standard.
47. The method of embodiment 46, wherein enriching or purifying the biological sample for lipids comprising the glycan comprises performing chromatography.
48. The method of embodiment 47, wherein the chromatography comprises hydrophilic interaction liquid chromatography (HILIC), liquid chromatography, solid-phase chromatography, column chromatography, affinity chromatography, ion exchange chromatography, or size exclusion chromatography, or combinations thereof.
49. The method of any one of embodiments 40-48, wherein the first label comprises an offset label.
50. The method of embodiment 49, wherein the offset label comprises an isotope label.
51. The method of any one of embodiments 40-50, wherein the second label comprises an isotope label.
52. The method of any one of embodiments 40-51, wherein the second label is added to the double-labeled lipid standard by enzymes or by organic synthesis.
53. The method of any one of embodiments 40-52, further comprising generating the double-labeled lipid standard.
54. The method of embodiment 53, wherein generating the double-labeled lipid standard comprises adding the first label to a portion of the biological sample.
55. The method of embodiment 53 or 54, wherein generating the double-labeled lipid standard comprises adding the first label and the second label to the portion of the biological sample.
56. The method of any one of embodiments 53-55, wherein generating the double-labeled lipid standard comprises pooling the portion of the biological sample and additional biological samples for enriching or purifying glycolipids.
57. The method of any one of embodiments 40-56, wherein identifying or measuring, based on the double-labeled lipid standard, the endogenous lipid of the biological sample comprises performing mass spectrometry.
58. The method of any one of embodiments 40-57, wherein identifying or measuring, based on the double-labeled lipid standard, the endogenous lipid of the biological sample comprises comparing a mass spectrum of the endogenous lipid to a mass spectrum of the double-labeled lipid standard.
59. The method of any one of embodiments 40-58, further comprising contacting the biological sample with a particle to capture the endogenous lipid on the particle.
60. The method of embodiment 59, wherein the particle comprises a nanoparticle.
61. The method of any one of embodiments 40-60, further comprising adding the endogenous lipid comprising glycans to a mass spectrometry library.
62. The method of any one of embodiments 40-61, further comprising obtaining a measurement of the endogenous lipid.
63. The method of any one of embodiments 40-62, wherein obtaining the measurement of the endogenous lipid comprises measuring a mass spectrum of the endogenous lipid.
64. The method of any one of embodiments 40-63, further comprising inputting the measurement of the endogenous lipid into a classifier to evaluate a biological state.
65. The method of embodiment 64, wherein the biological state comprises a healthy state.
66. The method of embodiment 64 or 65, wherein the biological state comprises a disease.
67. The method of embodiment 66, wherein the disease comprises a cancer.
68. The method of embodiment 67, wherein the cancer comprises pancreatic cancer.
69. The method of embodiment 67 or 68, wherein the cancer comprises lung cancer, breast cancer, colon cancer, liver cancer, or ovarian cancer.
70. A measurement method, comprising:
combining a biological sample with a double-labeled biomolecule standard comprising a biomolecule comprising a first label on a first portion of the biomolecule, and comprising a second label on a second portion of the biomolecule; and
identifying or measuring, based on the double-labeled biomolecule standard, an endogenous biomolecule of the biological sample, wherein the endogenous biomolecule comprises the first label on the first portion of the biomolecule or the second label on the second portion of the biomolecule.
71. The method of embodiment 70, wherein the endogenous biomolecule comprises a peptide or a lipid.

### Embodiments B

The following embodiments are not intended to be limiting in any way.
1. A device for enriching glycopeptides, comprising:
   a. a device body defining an interior volume comprising a distal opening and a proximal opening;
   b. chromatography material in the interior volume, the chromatography material comprising:
   c. a distal layer of stationary reverse phase chromatography material;
   d. a proximal layer of stationary reverse phase chromatography material; and
   e. a layer of hydrophilic interaction chromatography materials between said distal and proximal layers of reverse phase chromatography material;
wherein each of the said layers of chromatography materials comprises a homogenous composition, and wherein a liquid is able to flow through the volume in either direction.
2. The device of embodiment 1, wherein the device body comprises a pipette tip.
3. The device of embodiment 1, wherein the device body comprises a pipette.
4. The device of embodiment 1, wherein the device body comprises a chromatography column.
5. The device of embodiment 1, wherein the device body comprises a 96 well plate.
6. The device of embodiment 1, wherein the device body comprises multiple chromatography columns in series.
7. The device of embodiment 1, wherein the interior volume of the device body may be loaded with the liquid from the distal opening or the proximal opening.
8. The device of embodiment 1, wherein flowing a liquid through the chromatography material enriches glycopeptides of the liquid or removes non-glycopeptides from the liquid.
9. The device of embodiment 1, wherein the stationary reverse phase chromatography material comprises surface modified silica.
10. The device of embodiment 9, wherein the surface modified silica is modified with an unmodified alkyl ligand.
11. The device of embodiment 9, wherein the surface modified silica is modified by an alkyl ligand.
12. The device of embodiment 1, wherein the hydrophilic interaction chromatography material comprises unmodified silica.
13. The method of embodiment 1, wherein the hydrophilic interaction chromatography materials comprise cellulose.
14. The device of embodiment 1, wherein the hydrophilic interaction chromatography material comprises silica modified with a functional group.
15. The device of embodiment 14, wherein the functional group comprises a diol, cyano, amino, alkylamide, or a combination thereof.
16. The device of embodiment 14, wherein the functional group comprises a zwitterionic sulfbetaine.
17. The device of embodiment 1, further comprising a liquid flowing across the chromatography material either from the distal to the proximal opening or from the proximal to the distal opening, wherein the liquid comprises a biological sample or is from a biological sample.
18. The device of embodiment 1, comprising a liquid flowing the chromatography material wherein the liquid comprises an organic phase chromatography solvent.
19. The device of embodiment 1, comprising a liquid flowing the chromatography material wherein the liquid comprises an aqueous phase chromatography solvent.
20. The device of embodiment 1, wherein the device has a size suitable for large scale production of glycopeptides.
21. The device of embodiment 1 wherein the device is integrated into an automated platform.
22. The device of embodiment 1, wherein the device is connected directly to a detection device.
23. The device of embodiment 1, wherein the device is connected directly to a system comprising further methods of separation.
24. The device of embodiment 22, wherein the detection device is a mass spectrometer.
25. A kit comprising the device of any of the foregoing embodiments.
26. The kit of embodiment 25, wherein the device is fully assembled.
27. The kit of embodiment 25, wherein the kit comprises the individual components of the device in a form suitable for user assembly.
28. The kit of embodiment 25, further comprising instructions on the assembly or use of the device.
29. A method for enriching glycopeptides comprising:
   a. applying a biofluid comprising the glycopeptides onto a first stationary reverse phase chromatography material;
   b. washing the first stationary reverse phase chromatography material using an aqueous mobile phase;
   c. eluting the glycopeptides from the first stationary reverse phase chromatography material and applying it onto a distinct hydrophilic interaction chromatography material using an organic mobile phase;
   d. eluting the glycopeptides from the distinct hydrophilic interaction chromatography material and applying it onto a distinct second stationary reverse phase chromatography material using an aqueous mobile phase; and
   e. eluting the purified or enriched glycopeptides from the distinct second stationary reverse phase chromatography material using an organic mobile phase;
wherein the chromatography materials each comprise a homogenous composition.
30. The method of embodiment 29, wherein (a)-(e) take place within a single device.
31. The method of embodiment 30, wherein the device body defines an interior volume comprising a distal opening and a proximal opening.
32. The method of embodiment 30, wherein the device comprises a pipette tip.
33. The method of embodiment 30, wherein the device comprises a pipette.
34. The method of embodiment 30, wherein the device comprises a chromatography column.
35. The method of embodiment 30, wherein the device comprises a 96 well plate.
36. The method of embodiment 30, wherein the device comprises multiple chromatography columns in series.
37. The method of embodiment 29, wherein the stationary reverse phase chromatography material comprises surface modified silica.
38. The method of embodiment 37, wherein the surface modified silica is modified with an unmodified alkyl ligand.
39. The method of embodiment 37, wherein the surface modified silica is modified by an alkyl ligand.
40. The method of embodiment 29, wherein the hydrophilic interaction chromatography materials comprise cellulose.
41. The method of embodiment 29, wherein the hydrophilic interaction chromatography materials comprise unmodified silica.
42. The method of embodiment 29, wherein the hydrophilic interaction chromatography materials is silica modified with a functional group.
43. The method of embodiment 42, wherein the functional group comprises diols, cyanos, aminos, alkylamide, or a mixture thereof.
44. The method of embodiment 42, wherein the functional group comprises a zwitterionic sulfbetaine.
45. The method of embodiment 29, wherein the biofluid comprises a biological sample or is from a biological sample.
46. The method of embodiment 29, wherein the biofluid is from a human.
47. The method of embodiment 29, wherein the biofluid is modified prior to enrichment.
48. The method of embodiment 29, wherein the biofluid comprises salts, buffers, proteins, peptides, protein digests, and nucleic acids.
49. The method of embodiment 29, wherein the organic mobile phase comprises acetonitrile.
50. The method of embodiment 29, wherein the organic mobile phase comprises methanol.
51. The method of embodiment 29, wherein the organic mobile phase comprises tetrahydrofuran.
52. The method of any one of embodiments 49-51, wherein the organic mobile phase further comprises water.
53. The method of any one of embodiment 49-51, wherein the organic mobile phase further comprises trifluoracetic acid.
54. The method of embodiment 29, wherein the aqueous mobile phase comprises water.
55. The method of embodiment 54, wherein the aqueous mobile phase further comprises a buffer system.
56. The method of embodiment 55, wherein the buffer system further comprises trifluoroacetic acid.
57. The method of embodiment 55, wherein the buffer system further comprises phosphate.
58. The method of embodiment 55, wherein the buffer system further comprises formic acid.
59. The method of embodiment 55, wherein the buffer system further comprises acetic acid.
60. The method of embodiment 29, wherein the method is used for large scale production of purified glycopeptides.
61. The method of embodiment 29, wherein the method is integrated into an automated platform.
62. The method of embodiment 29, wherein the enriched glycopeptide comprises a buffer that is different than the buffer of the biofluid.
63. The method of embodiment 29, wherein the enriched glycopeptide comprises a lower concentration of salts than the biofluid.
64. The method of embodiment 29, wherein the eluted solution in (c) comprises peptides.
65. The method of embodiment 64, wherein the peptides are enriched.
66. The method of embodiment 64, wherein the peptides are desalted.
67. The method of embodiment 29, wherein the device is connected directly to a detection device.
68. The method of embodiment 29, wherein the device is connected directly to a system comprising further methods of separation.
69. The method of embodiment 67, wherein the detection device is a mass spectrometer.
70. The method of embodiment 29, further comprising contacting the biofluid with particles prior to applying the biofluid onto the first stationary reverse phase chromatography material.
71. The method of embodiment 70, wherein applying the biofluid onto the first stationary reverse phase chromatography material comprises applying glycopeptides captured by the particles onto the first stationary reverse phase chromatography material.

What is claimed is:

1. A method for generating mass/charge data of mass-spectrometry standards of a plurality of glycan-modified macromolecules, comprising:
(a) obtaining a biological sample and subjecting said biological sample to enriching said plurality of glycan-modified macromolecules to generate a plurality of enriched glycan-modified macromolecules, wherein a glycan-modified macromolecule of said plurality comprises a macromolecular backbone and at least one glycan modified macromolecular residue;
(b) attaching a macromolecular backbone-linked mass tag or isotopic tag to a glycan-modified macromolecule of said plurality of enriched glycan-modified macromolecules;
(c) attaching a glycan-linked isotopic tag to said glycan-modified macromolecule from (b) of said plurality of enriched glycan-modified macromolecules to generate said glycan-modified macromolecular standards; and
(d) performing a mass spectrometry assay on said glycan-modified macromolecule standards to obtain a plurality of mass/charge ratios or abundances of an ionized species associated with said glycan-modified macromolecular standards.

2. The method of claim 1, wherein said plurality of glycan-modified macromolecules comprises glycolipids.

3. The method of claim 2, wherein said plurality of glycan-modified macromolecules comprises glycopeptides.

4. The method of claim 3, wherein (a) further comprises digesting said glycopeptides with an endoprotease having a defined residue specificity.

5. The method of claim 4, wherein said endoprotease comprises Trypsin, rLys-C, Lys-C, rAsp-N, or any combination thereof.

6. The method of any one of claims 1-5, wherein (a) further comprises subjecting said biological sample to hydrophobic interaction liquid chromatography (HILIC) or lectin affinity chromatography.

7. The method of any one of claims 1-6, wherein said macromolecular backbone-linked isotopic tag comprises an isobaric labeling reagent.

8. The method of claim 7, wherein said isobaric labeling reagent comprises: (i) an amine-, thiol-, or carbonyl-reactive moiety, and (ii) an ionizable moiety comprising a stable heavy isotope.

9. The method of claim 7 or 8, wherein said isobaric labeling reagent comprises a tandem mass tag (TMT) reagent or an isobaric tag for relative and absolute quantitation (iTRAQ) reagent.

10. The method of any one of claims 1-9, wherein said glycan-linked isotopic tag is specific for: (i) a disaccharide linkage type of said enriched glycan-modified macromolecules, or (ii) a monosaccharide of said glycan of said enriched glycan-modified macromolecules.

11. The method of any one of claims 1-10, wherein said glycan-linked isotopic tag comprises a naturally-occurring monosaccharide comprising a heavy isotope atom.

12. The method of any one of claims 1-11, wherein (c) further comprises treating said plurality of enriched glycan-modified macromolecules with a monosaccharide-specific or linkage-specific glycosidase to remove a predetermined monosaccharide from a glycan of said plurality of enriched glycan-modified macromolecules.

13. The method of claim 12, further comprising treating said plurality of enriched glycan-modified macromolecules with a linkage-specific glycosidase, wherein said linkage-specific glycosidase comprises a glycosidase specific for an α2-3 sialic acid linkage, a β1-4 galactose linkage, a β1-3 galactose linkage, an α1-2 fucose linkage, or an α1-3/4 fucose linkage.

14. The method of claim 13, wherein said linkage-specific glycosidase comprises α2-3 Neuraminidase S, β1-4 Galactosidase S, β1-3 Galactosidase S, α1-2 Fucosidase, or α1-3,4 Fucosidase, or any combination thereof.

15. The method of claim 11 or 12, further comprising treating said plurality of enriched glycan-modified macromolecules with a monosaccharide-specific glycosidase, wherein said monosaccharide comprises sialic acid, mannose, N-Acetylglucosamine, or N-Acetylgalactosamine.

16. The method of claim 15, wherein said glucosidase comprises α2-3,6,8 Neuraminidase, α1-2,3,6 Mannosidase, β-N-Acetylglucosaminidase S, or α-N-Acetylgalactosaminidase, or any combination thereof.

17. The method of any one of claims 1-16, wherein (c) further comprises treating said plurality of enriched glycan-modified macromolecules with a glycosyltransferase to attach said glycan-linked isotopic tag to a glycan of said enriched glycan-modified macromolecules.

18. The method of claim 17, wherein said glycosyltransferase is configured to perform an α2-6 sialylation, an α2-3 sialylation, a β1-4 galactosylation, a β1-3 galactosylation, an α1-2 fucosylation, an α1-3 fucosylation, a α1-4 fucosylation, a β1-3 N-acetylglucosaminylation, or a β1-6 N-acetylglucosaminylation, or any combination thereof.

19. The method of claim 17 or 18, wherein said glycosyltransferase comprises α2-6 sialyltransferase 1 (ST6Gal1), α2-3 sialyltransferase 3/4 (ST3Gal3, ST3Gal4), β1-4 Galactosyltransferase 1 (β4GalT1), β1-3 Galactosyltransferase 5 (β3GalT5), Fucosyltransferase 1 or 2 (FucT1 or FucT2), Fucosyltransferase 4 or 7 (FucT4 or FucT7), Fucosyltransferase 3 (FucT3), β1-3N-acetylglucosaminyltransferase 2 (B3GNT2), or β1-6 N-acetylglucosaminylation (GCNT2), or any combination thereof.

20. The method of any one of claims 12-19, wherein said glycan-linked isotopic tag corresponds to a heavy isotope labeled derivative of said predetermined monosaccharide.

21. The method of any one of claims 1-20, wherein said glycan-linked isotopic tag comprises sialic acid or a derivative thereof, galactose or a derivative thereof, fucose or a derivative thereof, N-acetylglucosamine or a derivative thereof, N-acetylgalactosamine or a derivative thereof, mannose or a derivative thereof, glucose or a derivative thereof, or any combination thereof.

22. The method of claim 21, wherein said isotopic tag comprises sialic acid or a derivative thereof, wherein said sialic acid or a derivative thereof comprises N-Acetylneuraminic acid (Neu5Ac), N-Glycolylneuraminic acid (Neu5Gc), or 2-keto-3-deoxy-D-glycero-D-galacto-nononic acid (KDN).

23. The method of any one of claims 11-22, wherein said glycan-linked isotopic tag comprises a naturally-occurring monosaccharide comprising one, two, three, four, five or six ¹³C or ¹⁶O atoms.

24. The method of any one of claims 1-23, wherein said plurality of mass/charge ratios or abundances of an ionized species associated with said glycan-modified macromolecular standards comprise mass/charge ratios of precursor ions of said fragments of said glycan-modified macromolecular standards.

25. The method of any one of claims 1-24, wherein said plurality of mass/charge ratios or abundances of an ionized species associated with said glycan-modified macromolecular standards comprise mass/charge ratios of product ions of said fragments of said glycan-modified macromolecular standards.

26. The method of any one of claims 1-25, further comprising attaching a second macromolecular backbone-linked isotopic tag distinguishable in mass from said macromolecular backbone-linked isotopic tag in conjunction with a second glycan-linked isotopic tag to a second glycan-modified macromolecule of said plurality of enriched glycan-modified macromolecules.

27. The method of any one of claims 12-26, further comprising installing said second glycan-linked isotopic tag via treatment with a second linkage-specific glycosidase or second glycosyltransferase different to said linkage-specific glycosidase or said glycosyltransferase.

28. The method of claim 26 or 27, wherein said second glycan-linked isotopic tag is different to said glycan-linked isotopic tag.

29. The method of claim 28, wherein said second glycan-linked isotopic tag is not an isobar of said glycan-linked isotopic tag.

30. The method of any one of claims 1-25, further comprising pooling said glycan-modified macromolecule standards with a glycan-modified macromolecule of a second plurality of enriched glycan-modified macromolecules that has been labeled with a second macromolecular backbone-linked isotopic tag distinguishable in mass from said macromolecular backbone-linked isotopic tag, and obtaining a plurality of mass/charge ratios or abundances of a an ionized species associated with fragments of said glycan-modified macromolecule of said second plurality of enriched glycan-modified macromolecules.

31. The method of claim 30, further comprising pooling said glycan-modified macromolecule standards with a glycan-modified macromolecule of a third plurality of enriched glycan-modified macromolecules that has been labeled with a third macromolecular backbone-linked isotopic tag distinguishable in mass from said macromolecular backbone-linked isotopic tag and said second macromolecular backbone-linked isotopic tag, and obtaining a plurality of mass/charge ratios or abundances of an ionized species associated with of said glycan-modified macromolecule of said third plurality of enriched glycan-modified macromolecules.

32. The method of claim 26 or 31, wherein said second plurality of enriched glycan-modified macromolecules and said third plurality of enriched glycan-modified macromolecules are derived from distinct second and third biological samples, respectively.

33. The method of claim 28, comprising comparing an intensity of a mass/charge peak corresponding to a glycan-modified macromolecule of said second- and third-pluralities of enriched glycan-modified macromolecules, thereby identifying a difference in glycan abundance in said second and third biological samples.

34. The method of claim 33, further comprising using a mass offset of a glycan-modified macromolecule standard of said glycan-modified macromolecule standards to identify a site-specific glycan modification of a macromolecule corresponding to said difference in glycan abundance in said second and third biological samples.

35. A set of mass spectrometry standards of a plurality of glycan-modified macromolecules, wherein a glycan-modified macromolecule of said plurality comprises a macromolecular backbone and at least one glycan modified macromolecular residue, comprising:
a plurality of glycan-modified fragments of said glycan-modified macromolecules, wherein said plurality of glycan-modified fragments comprise:
(a) a macromolecular backbone-linked mass tag, isotopic tag, or isobaric tag; and
(b) a glycan-linked isotopic tag.

36. The set of mass spectrometry standards of claim 35, wherein said glycan-modified macromolecules comprise glycolipids.

37. The set of mass spectrometry standards of claim 35, wherein said glycan-modified macromolecules comprise glycoproteins.

38. The set of mass spectrometry standards of claim 37, wherein said plurality of glycan-modified fragments comprise fragments generated by treatment with an endoproteinase.

39. The set of mass spectrometry standards of claim 38, wherein said endoproteinase comprises Trypsin, rLys-C, Lys-C, rAsp-N, chymotrypsin, Glu-C, or any combination thereof.

40. The set of mass spectrometry standards of any one of claims 35-39, wherein said plurality of glycan-modified fragments comprise: (i) peptides with C-terminal arginine or lysine; (ii) peptides with C-terminal lysine; (iii) peptides with C-terminal arginine; (iv) peptides with C-terminal tyrosine, phenylalanine, or tryptophan; or (v) peptides with C-terminal glutamate.

41. The set of mass spectrometry standards of any one of claims 33-40, wherein said macromolecular backbone-linked isotopic tag comprises an isobaric labeling reagent.

42. The set of mass spectrometry standards of claim 41, wherein said isobaric labeling reagent comprises: (i) an amine-, thiol-, or carbonyl-reactive moiety and (ii) an ionizable moiety comprising a stable heavy isotope.

43. The set of mass spectrometry standards of claim 41 or 42, wherein said isobaric labeling reagent comprises a tandem mass tag (TMT) reagent or an isobaric tag for relative and absolute quantitation (iTRAQ) reagent.

44. The set of mass spectrometry standards of any one of claims 33-43, wherein said glycan-linked isotopic tag is specific for: (i) a disaccharide linkage type of said enriched glycan-modified macromolecules, or (ii) a monosaccharide of said glycan of said enriched glycan-modified macromolecules

45. The set of mass spectrometry standards of any one of claims 33-44, wherein said glycan-linked isotopic tag corresponds to a heavy isotope labeled derivative of a predetermined monosaccharide of a glycan attached to said glycan-modified macromolecules.

46. The set of mass spectrometry standards of any one of claims 33-45, wherein said glycan-linked isotopic tag comprises sialic acid or a derivative thereof, galactose or a derivative thereof, fucose or a derivative thereof, N-acetylglucosamine or a derivative thereof, N-acetylgalactosamine or a derivative thereof, mannose or a derivative thereof, glucose or a derivative thereof, or any combination thereof.

47. The set of mass spectrometry standards of claim 46, wherein said isotopic tag comprises sialic acid or a derivative thereof, wherein said sialic acid or a derivative thereof comprises N-Acetylneuraminic acid (Neu5Ac), N-Glycolylneuraminic acid (Neu5Gc), or 2-keto-3-deoxy-D-glycero-D-galacto-nononic acid (KDN).

48. The set of mass spectrometry standards of any one of claims 33-47, wherein said glycan-linked isotopic tag comprises a naturally-occurring monosaccharide comprising one, two, three, four, five or six ¹³C or ¹⁶O atoms.

49. The method of claim 6, further comprising digesting said biological sample with a protease prior to (a).

50. The method of claim 6, further comprising subjecting said biological sample to a reverse-phase liquid chromatography.

51. The method of claim 50, wherein subjecting said biological sample to said reverse-phase liquid chromatography occurs before or substantially at the same time as subjecting said biological sample to said HILIC or lectin affinity chromatography.

52. The method of claim 50, wherein subjecting said biological sample to said biological sample to said HILIC or lectin affinity chromatography occurs before or substantially at the same time as subjecting said reverse-phase liquid chromatography.

53. The method of claim 50, wherein said biological sample is not dried and is not further reconstituted prior to subjecting to reverse-phase liquid chromatography or HILIC.

54. The method of claim 51, wherein the reverse phase liquid chromatography and HILIC is configured to be deposited in the same device.

55. The method of claim 50, wherein said reverse-phase liquid chromatography comprises a non-polar stationary phase selected from the group consisting of C-18, C-12, C-8, C-3, phenyl, biphenyl, and any combinations thereof.

56. The method of claim 50, further comprising subjecting said biological sample to a metal oxide layer.

57. The method of claim 56, further comprising separation peptides, glycopeptides, and phosphopeptides from each other.

58. The method of claim 6, wherein said HILIC comprises one or more materials selected from the group consisting of cellulose, unmodified silica, silica modified with diols, silica modified with cyanos, silica modified with aminos, silica modified with a zwitterionic sulfobetaine, silica modified with alkylamides, and any combinations thereof.

59. The method of claim 6, further comprising separating peptides from glycopeptides.

60. A method for using a classifier capable of distinguishing a subject with a disease state, comprising:
a) adding the set of mass spectrometry standards of any one of claims 35-41 to a biological sample from said subject to generate a mixed biological sample;
b) subjecting said mixed biological sample to enrichment of PTM-modified macromolecules to generate an enriched mixed biological sample comprising PTM-modified macromolecules;
c) performing a mass spectrometry assay on said enriched mixed sample to obtain a plurality of mass/charge ratios and intensities corresponding to PTM-modified macromolecules of said enriched mixed biological sample; and
d) applying a trained machine learning classifier to said plurality of mass/charge ratios and intensities corresponding to PTM-modified macromolecules of said enriched mixed biological sample to obtain an output classification of whether said biological sample from said subject is associated with said disease state.

61. The method of claim 60, wherein said mass spectrometry assay is an LC/MS/MS assay.

62. The method of claim 60 or 61, wherein said classifier is selected from the group consisting of an artificial neural network, a support vector machine, a linear model, a non-linear model, a parametric model, a non-parametric model, a Bayesian model, a gaussian process, a binary classifier, a multilabel classifier, a non-binary classifier, a deep neural network, an ensemble method, a tree based model, a clustering method, a Markov model, and a combination thereof.

63. The method of claim 62, wherein said classifier comprises a linear model.

64. The method of claim 63, wherein said linear model comprises a ridge classifier, a stochastic gradient classifier, a passive aggressive classifier, or a perceptron.

65. The method of claim 62, wherein said classifier comprises a non-linear model.

66. The method of claim 65, wherein said non-linear model comprises a logistic regression model, a naïve bayes method, a kernel support vector machine, or a k nearest neighbor method.

67. The method of claim 62, wherein said classifier comprises an ensemble model.

68. The method of claim 67, wherein said ensemble model comprises a forest method, a random forest method, an extra trees classifier, an adaboost method, gradient boost method, or a voting classifier.

69. The method of claim 62, wherein said classifier comprises an artificial neural network.

70. The method of claim 62, wherein said classifier comprises a deep neural network model.

71. The method of claim 70, wherein said deep neural network is trained in an unsupervised setting, a supervised setting, a semi-supervised setting, or a self-supervised setting.

72. The method of claim 62, wherein said classifier uses a dimension reduction analysis.

73. The method of claim 72, wherein said dimension reduction analysis is selected from the group consisting of a principal component analysis, an independent component analysis, a linear discriminant analysis, a non-negative matrix factorization, a truncated singular value decomposition, a variational autoencoder, a transformer model, a u-net, a generative adversarial network, and any combination thereof.

74. The method of any one of claims 60-73, wherein said PTM-modified macromolecule comprises a macromolecule modified by glycosylation, ubiquitination, phosphorylation, acetylation, or any combination thereof.

75. The method of claim 74, wherein said PTM-modified macromolecule is a glycan-modified macromolecule.
